SEQUENCE LISTING (60 sequences)

SEQ ID NO: 1 - DNA, 1443 bp, synthetic construct
  misc_feature 1..1443: note = Codon-optimized coding sequence
  source 1..1443: mol_type = other DNA
atgaacgccagcgagtttagaaggagaggcaaggagatggtggattacatggccaattatatggagggcatcgaaggcagacaggtgtatcctgacgtggaacctggatatctgagacctctgattcctgccgctgcccctcaggaacctgacacattcgaggacatcattaatgacgtggagaagatcatcatgcctggcgtgacccactggcacagcccttacttcttcgcctactttcccaccgccagcagctatcccgccatgctggccgacatgctgtgcggcgccatcggctgcatcggcttcagctgggccgccagccccgcctgcaccgagctggagaccgtgatgatggactggctgggcaagatgctggagctgcccaaggcctttctgaacgagaaggccggcgagggcggcggagtgattcagggatccgctagcgaagccacactggtggctctgctggctgccagaacaaaggtgattcacagactgcaggccgcttctcccgagctgacccaggccgccatcatggagaagctggtggcctatagcagcgatcaggcccacagcagcgtggagagagccggcctgatcggcggcgtgaagctgaaggccatccctagcgacggcaacttcgccatgcgcgccagcgccctgcaggaggccctggagcgcgacaaggccgccggcctgatccctttcttcatggtggccaccctgggcacaaccacctgctgcagcttcgacaacctgctggaggtgggacccatctgcaacaaggaggacatctggctgcacgtggacgccgcctacgccggcagcgccttcatctgccccgagttcaggcacctgctgaacggcgtggagttcgccgatagctttaacttcaatccccacaagtggctgctggtgaattttgactgcagcgccatgtgggtgaagaagaggaccgacctgaccggcgccttcaggctggatcccacctacctgaagcacagccaccaggacagcggcctgatcaccgactacaggcactggcagatccccctgggcaggagattccgcagcctgaagatgtggttcgtgttcagaatgtacggcgtgaagggcctgcaggcctacatcagaaagcacgtgcagctgagccacgagtttgaaagcctggtgagacaggatcctagatttgaaatctgtgtggaggtgatcctgggcctggtgtgttttagactgaagggaagcaataaagtgaacgaagccctgctgcagaggatcaatagcgccaagaagattcacctggtgccttgtcacctgagagacaagtttgtgctgagattcgccatttgttctagaaccgtggagagcgcccacgtgcagagagcctgggagcacatcaaggagctggccgccgacgtgctgagagccgagagggagtga

SEQ ID NO: 2 - DNA, 1443 bp, synthetic construct
  misc_feature 1..1443: note = CDS
  source 1..1443: mol_type = other DNA
atgaacgccagcgagttccgcagacggggcaaggagatggtggactacatggccaactacatggagggcattgagggcagacaggtgtaccccgacgtggagcccggctacctgagaccactcatccctgccgccgcccctcaggagcccgacaccttcgaggacatcattaacgacgtggagaagattattatgcccggcgtgacccactggcactccccttacttcttcgcctacttcccaacagcctctagctacccagccatgctcgccgacatgctgtgcggcgccattggctgcatcggcttctcttgggccgcctccccagcctgcacagagttagaaacagtgatgatggactggctgggcaagatgttggaactcccaaaggccttcctgaacgagaaggccggcgagggcggcggcgtgatccagggctctgccagcgaggccacactcgtggccctcctcgccgcccgcacaaaggtgatccacagactgcaggccgccagccctgagctgacacaggccgccattatggagaagctggtggcctactctagcgaccaggcccactcttctgtggagagagccggcctcattggcggcgtgaagctcaaggccatccctagcgacggcaacttcgccatgagagccagcgccctccaggaggcccttgaacgggacaaggccgccggcctgattcctttcttcatggtggccacactgggcacaaccacatgctgctctttcgacaacctcctagaagtgggccctatttgcaacaaggaggacatttggctgcacgtggacgccgcctacgccggctctgccttcatttgccccgagttcagacacctcctgaacggcgtggagttcgccgactctttcaacttcaacccacacaagtggctcctggtgaacttcgactgcagcgccatgtgggtgaagaagcgcaccgacctgaccggcgccttcagactggacccaacatacctgaagcactctcaccaggactccggcctgatcaccgactacagacactggcagattccactgggcagacggttccggtctctcaagatgtggttcgtgttccggatgtacggcgtgaagggcctccaggcctacattagaaagcacgtgcagctgtctcacgagttcgagtctctggtgcggcaggaccctagattcgagatttgcgtggaggtgattctcggcctcgtgtgcttcagactcaagggctctaacaaggtgaacgaggccctgctgcagcggattaactctgccaagaagattcacctggtgccttgccacctccgggacaagttcgtgctgcggttcgccatttgctcaaggacagtggagtctgcccacgtgcagagagcctgggagcacattaaggaactcgccgccgacgtgctgagagccgagcgcgagtga

SEQ ID NO: 3 - DNA, 1443 bp, synthetic construct
  misc_feature 1..1443: note = CDS
  source 1..1443: mol_type = other DNA
atgaacgccagcgagtttagaagacggggcaaagagatggtcgactacatggccaattacatggaaggcatcgagggcaggcaggtgtaccctgatgtggaacccggatatctgagacctctgattcccgctgcagcccctcaggagcctgacacattcgaggatatcatcaacgacgtggaaaaaatcatcatgcctggcgtcacccactggcactctccttacttctttgcctatttccctacagccagcagctaccccgccatgctggccgacatgctgtgtggcgccatcggctgcatcggcttcagctgggccgcctccccagcctgcaccgagctggaaaccgtgatgatggactggctgggaaagatgctggaactgcctaaggcctttctgaatgagaaggctggcgagggcggaggagtgatccagggctccgcttctgaggccaccctggttgctctgcttgccgccagaaccaaggtgatccacagactgcaggccgcttctccagagctgacccaggccgccatcatggaaaagctggtggcttacagcagcgaccaggcccattccagcgtggaacgggccggcctgatcggcggcgtgaagctcaaagccatccctagcgacggaaatttcgccatgcgggcctccgccctgcaagaggccctggaaagagataaggccgccggactcatcccgttcttcatggtggccacactgggtacaactacatgctgctcttttgacaacctgctggaagtgggccctatctgcaacaaagaggacatctggctgcacgtggacgccgcctacgccggctctgccttcatctgtcctgagttcagacatctgctgaacggcgtggagttcgctgatagcttcaacttcaacccccacaagtggctgctggtcaacttcgactgcagcgctatgtgggtgaaaaagcgcaccgacctgaccggcgcttttagactggaccccacctacctgaaacacagccaccaggatagcggcctgatcaccgattacagacactggcagattcccctgggcagacggttccggagcctgaagatgtggttcgttttcagaatgtacggcgtgaagggcctgcaagcctacatcagaaagcacgttcagctgagccacgagttcgagagcctggtgcggcaggaccctagatttgagatctgcgtggaagtgatcctgggcctcgtgtgcttccgcctgaagggcagcaacaaggtgaacgaggctctgctgcagcggatcaacagcgccaagaagatccacctggtgccttgtcacctgcgggacaagttcgtgctgagattcgcaatttgtagcagaaccgtggaaagcgcccacgtgcagagagcctgggagcacatcaaggaactggccgctgatgtgctgagagccgagcgggaatga

SEQ ID NO: 4 - DNA, 1443 bp, synthetic construct
  misc_feature 1..1443: note = CDS
  source 1..1443: mol_type = other DNA
atgaacgctagcgagttcagacggagaggcaaggagatggtggactacatggccaactacatggagggcatcgagggcagacaagtgtaccccgacgtggagcccgggtacctgagacccctgattcccgccgctgccccccaagagcccgacaccttcgaggacatcatcaacgacgtggagaagatcatcatgcccggcgtgacccactggcacagcccctacttcttcgcctacttccccaccgctagcagctaccccgccatgctggccgacatgctgtgcggggccatcggctgcatcggcttcagctgggccgctagccccgcctgcaccgagctggagaccgtgatgatggactggctgggcaagatgctggagctgcccaaggccttcctgaacgaaaaggctggcgaaggcgggggggtgatccaaggcagcgctagcgaggctacactggtggccctgctggccgctagaaccaaggtgatccacagactgcaagccgcttcccctgagctgacccaagccgccatcatggagaagctggtggcctacagcagcgaccaagcccacagcagcgtggagagagccggcctgatcggcggggtgaagctgaaggccatccctagcgacggcaacttcgccatgagagctagcgccctgcaagaggccctggagagagacaaggccgccggcctgatccccttcttcatggtggccaccctgggcaccacaacctgctgcagcttcgacaacctgctggaggtgggccccatctgcaacaaggaggacatctggctgcacgtggacgccgcctacgccggcagcgccttcatctgccccgagttcagacacctgctgaacggcgtggagttcgccgacagcttcaacttcaacccccacaagtggctgctggtgaacttcgactgcagcgccatgtgggtgaagaagagaaccgacctgaccggcgccttcagactggaccccacctacctgaagcacagccaccaagacagcggcctgatcaccgactacagacactggcagatccccctgggcagaagattcagaagcctgaagatgtggttcgtgttcagaatgtacggcgtgaagggcctgcaagcctacatcagaaagcacgtgcagctgagccacgagttcgagagcctggtgagacaagaccctagattcgagatctgcgtggaggtgatcctgggcctggtgtgcttcagactgaagggcagcaacaaggtgaacgaggccctgctgcagagaatcaacagcgccaagaagatccacctggtgccctgccacctgagagacaagttcgtgctgagattcgccatctgcagcagaaccgtggagagcgcccacgtgcagagagcctgggagcacatcaaggagctggccgccgacgtgctgagagccgagagagagtga

SEQ ID NO: 5 - DNA, 1443 bp, synthetic construct
  misc_feature 1..1443: note = CDS
  source 1..1443: mol_type = other DNA
atgaatgcctcagaattccgcaggaggggtaaggagatggtggactacatggccaattatatggaaggcatcgaaggtcggcaggtgtatcctgacgtggagcccggctacttgagacccctgattcccgccgcagccccccaggaaccggacacatttgaggatatcatcaatgacgtcgagaagatcatcatgccgggagtgactcattggcactcaccgtacttttttgcctattttccgactgcctcctcctatcctgccatgcttgcggacatgttgtgtggagcgatcggctgcatcggcttctcttgggcagcatcccccgcctgtaccgagctggaaaccgtcatgatggattggcttggaaagatgcttgagctgcccaaggcttttctgaatgaaaaggctggcgaggggggcggcgtgatccagggttctgcaagtgaagccactctggtcgcattgctggctgcccggacaaaagttattcacagactgcaggccgcttcacctgaactgacgcaagcagccatcatggaaaagctggtggcctattcttccgaccaggcccatagctctgttgagcgggctgggcttataggaggtgtgaaacttaaagctatcccttcagacggtaattttgccatgcgcgcttccgccctgcaggaggctctggaacgcgataaagctgccggcctcattcctttcttcatggttgccaccctcggtactactacctgttgcagctttgataacctgctggaggtgggccctatttgcaacaaagaggatatctggctgcacgtggacgcagcatacgctgggtctgccttcatctgcccggagtttcggcatctccttaatggagttgagttcgcagacagtttcaattttaatcctcacaagtggttgcttgtgaacttcgactgttctgcaatgtgggtgaagaaaaggactgacctgactggcgccttccggctcgacccaacttacctgaaacattctcaccaagactcaggactgattacagactatagacattggcagattcctctcggcagaaggttcaggagtctgaaaatgtggttcgtctttcggatgtacggagtgaaaggtctgcaggcctatatccgcaagcacgtgcaactttcccatgagttcgagtctctcgttcggcaggatcctcgctttgagatctgtgtggaggtcattctggggctggtgtgcttcaggctgaagggcagcaacaaagtgaatgaggctttgcttcagaggatcaactctgccaaaaagatccacttggtgccctgccatctgagggacaagttcgtcctgagattcgctatttgttccagaactgttgaaagtgcacacgtgcaacgggcgtgggagcacatcaaagaactggccgcagacgtgctgagggccgagagggagtga

SEQ ID NO: 6 - DNA, 1443 bp, synthetic construct
  misc_feature 1..1443: note = CDS
  source 1..1443: mol_type = other DNA
atgaacgccagcgagtttagaaggaggggaaaggagatggtggattacatggctaactacatggaggggatcgagggcagacaggtctaccctgatgtggagcctggctacctgaggcccctgatccctgccgccgccccacaggaaccagatacttttgaggatattattaacgatgtcgagaagattattatgccaggagtgacacactggcatagcccctacttctttgcttattttcccacagctagctcttaccctgcaatgttggctgatatgctgtgcggcgccattggatgcattggctttagttgggctgccagtcctgcctgcacagaattggagacagtgatgatggattggctgggaaagatgctggagctgcctaaggcctttctgaacgagaaggccggcgaggggggcggcgtgatccaggggagcgccagcgaggccaccctggtcgccctgctggccgcccggaccaaggtcattcacaggctgcaggccgccagccccgagctgacacaggccgccatcatggagaagctggtggcctacagctccgaccaggcccacagctccgtggagcgggccggcctgatcggcggcgtgaagctgaaggccattccctcagacgggaacttcgccatgcgggccagcgccctccaggaggccttggagagggataaggccgccggcctgatccccttctttatggtggctaccctggggacaaccacatgctgctccttcgataacctgctggaggtggggcccatctgcaacaaggaggacatctggctgcacgtggatgccgcctacgccgggtccgccttcatttgccccgagttccggcacctgctgaacggcgtggagttcgccgatagcttcaacttcaacccccacaagtggctgctggtgaacttcgactgcagcgccatgtgggtgaagaagaggaccgacctgaccggcgcctttaggctcgaccccacatacctgaagcacagccaccaggactccgggctgatcacagattacaggcactggcagatccccctcggcaggcggttccggtccctgaagatgtggttcgtgttccggatgtacggggtgaaggggctgcaggcctacatcaggaagcacgtccagctcagccacgagttcgagagcctggtgaggcaggacccccggttcgagatctgcgtggaggtcatcctggggctggtgtgtttccggctcaagggctccaacaaggtgaacgaggccctcctccagcggattaactccgccaagaagatccacctggtcccctgccacctgagggataagttcgtgctgcggttcgccatttgcagcaggacagtggagagcgcccacgtgcagcgggcctgggagcacattaaggagctggccgccgatgtgctgagggccgagagggagtga

SEQ ID NO: 7 - DNA, 1443 bp, synthetic construct
  misc_feature 1..1443: note = CDS
  source 1..1443: mol_type = other DNA
atgaacgccagcgagttcagacgcaggggcaaggagatggtggactacatggccaactacatggagggcattgagggcagacaggtgtaccccgacgtggagcccggctacctcagaccactcatcccagccgccgcccctcaggagcccgacacattcgaggacattattaacgacgtggagaagattattatgcccggcgtgacccactggcactccccttacttcttcgcctacttcccaacagcctctagctacccagccatgctcgccgacatgctgtgcggcgccatcggctgcattggcttctcttgggccgcctccccagcctgcaccgagttagagacagtgatgatggactggctgggcaagatgcttgagctacctaaggccttcctgaacgagaaggccggcgagggcggcggcgtgatccagggctctgccagcgaggccacactggtggccctcctcgccgcccgcacaaaggtgatccacagactccaggccgcctccccagagctgacacaggccgccatcatggagaagctggtggcctactctagcgaccaggcccactcttctgtggagagagccggcctgattggcggcgtgaagctgaaggccattccatctgacggcaacttcgccatgagagccagcgccctccaggaggcccttgaacgggacaaggccgccggcctgattcctttcttcatggtggccacactgggcaccacaacatgctgctctttcgacaacctcctggaggtgggcccaatttgcaacaaggaggacatttggctgcacgtggacgccgcctacgccggctctgccttcatttgccctgagttcagacacctcctgaacggcgtggagttcgccgactctttcaacttcaacccacacaagtggctcctggtgaacttcgactgctctgccatgtgggtgaagaagcgcaccgacctgaccggcgccttcagactggacccaacatacctcaagcactctcaccaggactccggcctcatcaccgactacagacactggcagattccactgggcagacggttccggagtctcaagatgtggttcgtgttccggatgtacggcgtgaagggcctccaggcctacattagaaagcacgtgcagctgtctcacgagttcgagtctctcgtgagacaggaccctaggttcgagatttgcgtggaggtgattctcggcctggtgtgcttcagactcaagggctctaacaaggtgaacgaggccctccttcagcggattaactctgccaagaagatccacctcgtgccttgccacctgcgggacaagttcgtgctgcggttcgccatttgctcccgtacagtggagtctgcccacgtgcagcgcgcctgggagcacattaaggaactcgccgccgacgtgctgagagccgagcgcgagtga

SEQ ID NO: 8 - DNA, 1443 bp, synthetic construct
  misc_feature 1..1443: note = CDS
  source 1..1443: mol_type = other DNA
atgaacgccagcgagttccggagaagggggaaggagatggtggactacatggccaactacatggaggggatcgaggggagacaggtgtaccccgacgtggagcccgggtacctgagacccctgattcccgccgctgctccccaggaaccagacaccttcgaggacatcattaacgacgtggagaagatcattatgccaggcgtgacccactggcatagcccctacttctttgcctacttccccacagcctcctcataccccgccatgctggccgacatgctgtgcggcgccattggctgcatcggcttcagctgggctgcttctcccgcttgcaccgagctggagaccgtgatgatggactggctgggcaagatgctggagctgcccaaggccttcctgaacgagaaggccggcgagggcggaggcgtgatccagggatccgccagcgaggccaccctggtggctctgctggccgctaggaccaaggtgatccacagactgcaggccgccagtcccgagctgacccaggctgctatcatggagaagctggtggcctactcctccgaccaggcccacagctccgtggagagagccggactgatcggcggcgtgaaactgaaggccattccctccgacggcaacttcgctatgcgggccagcgccctgcaggaggctctggaaagggacaaggccgccggcctgattcccttcttcatggtggctacactgggcaccaccacctgctgctcctttgataacctgctggaggtgggacccatttgcaacaaggaagacatttggctgcacgtggatgctgcctacgccggcagcgcctttatttgccccgagttcagacacctgctgaatggcgtggagttcgctgactccttcaacttcaacccccacaagtggctgctggtgaacttcgattgttctgccatgtgggtgaagaagaggaccgacctgaccggagccttccggctggaccctacctacctgaaacactcccaccaggactccggcctgattaccgactacagacactggcagatccccctgggcagaagattcagaagcctgaaaatgtggtttgtgttcagaatgtacggcgtgaaaggcctgcaggcctacatcaggaagcacgtgcagctgagccacgaattcgagagcctggtgagacaggaccccagatttgaaatctgcgtggaggtgattctgggcctggtgtgtttcagactgaagggaagcaacaaggtgaacgaggccctgctgcagaggatcaactcagccaagaagatccacctggtgccctgtcacctgagagacaagttcgtgctgagattcgccatctgctctagaacagtggagagcgcccacgtgcagagagcctgggagcacatcaaggagctggccgccgacgtgctgagagccgaaagagagtga

SEQ ID NO: 9 - DNA, 1443 bp, synthetic construct
  misc_feature 1..1443: note = CDS
  source 1..1443: mol_type = other DNA
atgaacgccagcgagtttcggcggagaggcaaagaaatggtggactacatggccaactacatggaaggcatcgagggcagacaggtgtaccccgatgtggaacctggctacctgaggcctctgattcctgccgctgctccccaagagcctgacaccttcgaggacatcatcaacgacgtggaaaagatcatcatgcccggcgtgacccactggcacagcccctactttttcgcctactttcccaccgccagcagctaccctgctatgctggctgatatgctgtgtggcgccatcggctgcatcggcttttcttgggctgcctctcctgcctgcaccgagctggaaaccgtgatgatggattggctgggcaagatgctggaactgcccaaggccttcctgaacgagaaagctggcgaaggcggcggagtgattcagggatctgcctctgaagccacactggtggctctgctggccgccagaacaaaagtgatccacagactgcaggccgcctctccagaactgacacaggccgccatcatggaaaagctggtggcctacagcagcgatcaggcccacagctctgtggaaagagccggactgattggcggcgtgaagctgaaggccattcctagcgacggcaacttcgccatgagagcctctgctctgcaagaggccctggaaagagataaggccgctggactgatccccttcttcatggtggctaccctgggcaccaccacctgttgcagcttcgacaacctgctggaagtgggccccatctgcaacaaagaggacatctggctgcacgtggacgccgcctatgccggctctgcctttatctgccccgagttcagacatctgctgaacggcgtggaattcgccgacagcttcaacttcaaccctcacaagtggctgctggtcaacttcgactgcagcgctatgtgggtcaagaagcggaccgatctgaccggcgccttcagactggatcccacctacctgaagcactcccaccaggatagcggcctgatcaccgactaccggcactggcagattcctctgggcagaagatttcggagcctgaagatgtggttcgtgttccggatgtatggcgtgaagggcctccaggcctacatcagaaaacatgtgcagctgagccacgagttcgagagccttgtcagacaggaccccagattcgagatctgcgtggaagtgatcctgggcctcgtgtgctttagactgaagggcagcaacaaagtgaacgaggccctgctgcagcggatcaacagcgccaaaaagatccacctggtgccttgccacctgagagacaagttcgtgctgagattcgccatctgcagccggacagtggaaagcgcccatgtgcagagagcctgggagcacatcaaagaactggccgccgatgtgctgagggccgagagagaatga

SEQ ID NO: 10 - DNA, 1443 bp, synthetic construct
  misc_feature 1..1443: note = CDS
  source 1..1443: mol_type = other DNA
atgaacgcaagtgaattccgaaggagagggaaggagatggtggattacgtggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagtag

SEQ ID NO: 11 - DNA, 1443 bp, Homo sapiens
  source 1..1443: mol_type = genomic DNA
atgaacgcaagtgaattccgaaggagagggaaggagatggtggattacatggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagtag

SEQ ID NO: 12 - DNA, 1611 bp, synthetic construct
  misc_feature 1..1611: note = CDS
  source 1..1611: mol_type = other DNA
atggagttcagctctcccagcagagaagaatgtcctaagcctctgagcagggtgtccatcatggccggcagcctgaccggactcctgctcctgcaggccgtgagctgggccagcggcgccaggccttgcattcccaagagcttcggctacagcagcgtggtgtgcgtgtgcaacgccacctactgcgacagcttcgatcctcctacctttcctgccctgggaacattctcccgctacgagagcaccaggagcggccggagaatggagctgagcatgggacccatccaggccaaccacacaggaacaggcctcctgctcaccctgcagcctgagcaaaaatttcagaaggtgaagggcttcggcggcgccatgaccgacgctgccgctctgaacatcctggccctgagccctcctgcccagaacctcctgctcaagagctacttcagcgaggagggcatcggctacaacatcatcagagtgcccatggccagctgcgactttagcattaggacatatacctacgccgatacacctgatgacttccagctgcataactttagcctgcctgaggaggataccaagctgaaaatccctctgatccatcgcgccctgcagctggcccagagacctgtgtccctgctggccagcccttggacaagccctacatggctgaagaccaacggcgctgtgaatggcaaaggcagcctgaaaggacagcctggcgatatctatcaccagacctgggccaggtattttgtgaaatttctggatgcctacgccgagcacaagctgcagttctgggccgtgaccgctgagaatgaaccttctgccggactgttgagcggctatccctttcagtgtctgggattcacccctgagcaccagcgcgacttcatcgcccgcgacctgggccctaccctggccaacagcacccaccacaacgtgcgcctgctgatgctggacgatcagagactgctgctgccccactgggccaaggtggtgctgaccgaccctgaggccgccaagtacgtgcacggcatcgccgtgcactggtacctggacttcctggctcctgccaaggccaccctgggcgagacccacaggctgttccctaacaccatgctgttcgccagcgaggcctgcgtgggctccaagttctgggagcagtccgtgaggctgggaagctgggacaggggcatgcagtacagccacagcatcatcaccaacctgctgtaccacgtggtgggctggaccgactggaacctggccctgaaccctgagggcggcccaaactgggtgcgcaacttcgtggacagccctatcatcgtggacatcaccaaggacaccttctacaagcagcccatgttctaccacctgggccacttcagcaagtttattcctgagggcagccaaagggtgggactggtggccagccagaaaaatgacctggacgccgtggctctgatgcatcctgatggcagcgccgtcgtggttgtcctgaaccgcagcagcaaggacgtgcccctgaccatcaaggatcctgccgtgggcttcctggagaccatcagccctggctacagcatccacacctacctgtggaggagacagtga

SEQ ID NO: 13 - DNA, 1611 bp, synthetic construct
  misc_feature 1..1611: note = CDS
  source 1..1611: mol_type = other DNA
atggagttcagctccccatctagggaggagtgccctaagccactgagtcgcgtgtctattatggccggctctctgacaggcctcctgctcctgcaggccgtgtcttgggcctctggcgccagaccttgcatcccaaagtctttcggctactctagcgtggtgtgcgtgtgcaacgccacatactgcgactctttcgacccaccaaccttccccgccctcggcaccttctcccggtacgagtctacacggtccggcaggcggatggagctgtccatgggcccaattcaggccaaccacacaggcacaggcctgctcctgacactgcagccagagcagaagttccagaaggtgaagggcttcggcggcgccatgaccgacgccgccgccctcaacattctcgccctgtccccacccgcccagaacctgctgctcaagtcttacttcagcgaggagggcattggctacaacatcatccgggtgcctatggcctcttgcgacttcagcattagaacatacacatacgccgacacacctgacgacttccagctgcacaacttcagcctccccgaggaggacacaaagctcaagattccactgattcaccgcgccctgcagctggcccagagaccagtgtctctgctcgcctccccttggacatctccaacatggctgaagacaaacggcgccgtgaacggcaagggctctctgaagggccagccaggcgacatttaccaccagacatgggcccggtacttcgtgaagttcctcgacgcctacgccgagcacaagctgcagttctgggccgtgacagccgagaacgagcctagcgccggcctgctgtccggctaccctttccagtgcctcggcttcacccctgagcaccagcgggacttcattgcccgggacctcggccctacactcgccaactctacccaccacaacgtgagactcctgatgctggacgaccagagactgctgctccctcactgggccaaggtggtgctcaccgaccccgaggccgccaagtacgtgcacggcattgccgtgcactggtacctcgacttcctcgccccagccaaggccacactcggcgagacccacagactgttccctaacaccatgctgttcgcctctgaggcctgcgtgggcagcaagttctgggagcagtctgtgagactcggctcttgggaccgcggcatgcagtactctcactccattattacaaacctgctgtaccacgtggtgggctggaccgactggaacctcgccctgaaccccgagggcggccctaactgggtgcggaacttcgtggactctcctatcattgtggacattacaaaggacacattctacaagcagcctatgttctaccacctcggccacttctctaagttcatccccgagggctctcagagagtgggcctggtggcctctcagaagaacgacctggacgccgtggccctgatgcaccccgacggcagcgccgtggtggtggtgctcaacagatcgagcaaggacgtgccactgacaattaaggacccagccgtgggcttccttgaaacaatttctccaggctactctattcacacatacctgtggcggagacagtga

SEQ ID NO: 14 - DNA, 1611 bp, synthetic construct
  misc_feature 1..1611: note = CDS
  source 1..1611: mol_type = other DNA
atggagttcagcagccccagccgggaagagtgccctaagcctctgagcagagtgtccatcatggccggttccctgaccggcctgctgcttctgcaagccgtgtcttgggccagcggagccagaccctgcatcccaaagtccttcggctacagctctgtggtctgcgtgtgcaacgccacctactgcgacagcttcgaccctccaaccttccctgccttgggaaccttcagcagatatgagagcacaagaagcggcagaagaatggaactgagcatgggccctattcaggccaatcacacaggcaccggcttactgctgacactgcagcctgagcagaagttccagaaagtgaaaggcttcggcggcgccatgaccgacgccgctgctctgaatatcctggctctgagcccacctgctcagaacctgctgctgaaaagctacttttctgaggaaggcatcggatacaacatcatcagagtgcctatggcctcttgtgacttcagcatccggacctacacctatgccgacacacctgatgactttcagctgcacaatttcagcctgcccgaggaagatacaaagctgaagatccccctgatccaccgggccctgcagctggcccagagacctgtgtccctgctggcttctccttggaccagccctacctggctcaaaacaaacggcgccgtgaacggcaagggctctctgaagggccagcctggagatatctaccaccaaacatgggccagatacttcgtgaagttcctggacgcctacgccgaacacaagctgcagttctgggcagtgaccgccgagaacgagccttctgccggactgctgagcggctaccccttccaatgtctgggctttacccctgaacaccagcgcgacttcatcgccagagatctgggccctacactggccaacagcacccaccacaacgtgcggctgctcatgctggacgaccagagactgctgcttcctcactgggccaaggtggtcctgaccgaccctgaagccgctaagtacgtgcacggcatcgccgttcactggtacctggattttctggcccctgccaaggccacgctgggcgagacacacagactgtttccaaataccatgctgttcgccagcgaggcatgcgtggggagcaaattctgggagcagagcgtgcggctgggctcctgggacagaggcatgcaatacagccactctatcatcaccaacctgctgtaccatgtggtgggctggacagactggaacctggccctgaaccccgagggaggccccaactgggtcaggaacttcgtggatagccccatcatcgttgacatcaccaaggacaccttctacaagcagcccatgttctaccatctgggacacttctccaagtttatccctgagggctcccagcgggtgggcctggtggccagccagaagaacgacctggatgccgtggctctgatgcaccctgacggcagcgctgtggtggtggtgctgaacagatcctccaaggatgtgcccctgactatcaaggaccccgccgtgggattcctggaaaccatcagccctggctatagcattcacacctacctgtggcggagacagtga

SEQ ID NO: 15 - DNA, 1611 bp, synthetic construct
  misc_feature 1..1611: note = CDS
  source 1..1611: mol_type = other DNA
atggagttcagctcccctagcagagaggagtgccccaagcccctgagcagagtgtccattatggctggcagcctgaccggcctgctgctcctgcaagccgtgagctgggctagcggcgctagaccctgcatccccaagagcttcggctacagcagcgtggtgtgcgtgtgcaacgccacctactgcgacagcttcgacccccccaccttccccgccctgggcaccttcagcagatacgagagcacaagaagcggcagaagaatggagctgagcatgggccccatccaagccaaccacaccggcaccggcctgctcctgacactgcagcccgagcagaagtttcagaaggtgaagggctttgggggcgccatgaccgacgccgctgccctgaatatcctggccctgagcccccccgctcagaacctgctcctgaagagctacttcagcgaggagggcatcggctacaacatcatcagagtgcccatggctagctgcgacttcagcatcagaacctacacctacgccgacacccccgacgactttcagctgcacaacttcagcctgcccgaggaggacaccaagctgaagatccccctgatccacagagccctgcagctggctcagagacccgtgagcctgctggctagcccctggacaagccccacctggctgaagaccaacggcgccgtgaacggcaagggcagcctgaaggggcagcccggcgacatctaccatcagacctgggctagatacttcgtgaagttcctggacgcctacgccgagcacaagctgcagttttgggctgtgaccgccgagaacgagcctagcgccggcctgctgagcggctacccctttcagtgcctgggcttcacccccgagcatcagagagacttcatcgctagagacctgggccccaccctggccaacagcacccaccacaacgtgagactgctgatgctggacgatcagagactgctcctgccccactgggccaaggtggtgctgaccgaccccgaggccgccaagtacgtgcacggcatcgccgtgcactggtacctggacttcctggcccccgccaaggccaccctgggcgagacccacagactgttccccaacaccatgctgttcgctagcgaggcctgcgtgggcagcaagttctgggagcagagcgtgagactgggcagctgggacagaggcatgcagtacagccacagcatcatcaccaacctgctgtaccacgtggtgggctggaccgactggaacctggccctgaaccccgagggcggccccaactgggtgagaaacttcgtggacagccccatcatcgtggacatcaccaaggacaccttctacaagcagcccatgttctaccacctgggccacttcagcaagttcatccccgagggcagccaaagagtgggcctggtggctagccaaaagaacgacctggacgccgtggccctgatgcaccccgacggcagcgccgtggtcgtggtgctgaacagaagcagcaaggacgtgcccctgaccatcaaggaccccgccgtgggcttcctggagaccatcagccccggctacagcatccacacctacctgtggagaagacagtga

SEQ ID NO: 16 - DNA, 1611 bp, synthetic construct
  misc_feature 1..1611: note = CDS
  source 1..1611: mol_type = other DNA
atggaatttagctcacctagccgggaggaatgcccaaaacccctctcccgggtgtccattatggctggaagcctcacaggccttctgctcttgcaagccgtcagctgggcctctggggcccggccatgtattccaaaatccttcggttactcatctgtggtctgtgtgtgtaatgcaacgtactgcgactcattcgatccccccacattcccagccctggggacgttttcccgctacgaaagcactcggtctggcaggcggatggaactgtccatgggtcccatccaggctaaccacactggaactggactgctgctgacgctgcagcccgaacagaagtttcagaaggttaagggtttcggcggcgccatgactgacgccgcggctctgaatatcctggcgctgagcccacctgcacagaatctgctgctgaaaagctacttcagtgaggaagggatcggatacaatattatcagggtacctatggcaagctgtgatttttctatccggacatacacgtacgctgacactccagatgatttccagctgcataacttttctctccccgaggaggatacaaagctgaagatccctctgatccatcgcgccctccagctggcccagcgccccgtatctcttcttgcgagcccttggacaagtcctacatggctgaagactaatggtgccgttaatggaaagggcagtttgaaaggccagccaggtgacatctatcaccagacatgggcccggtactttgtaaagttcctcgatgcgtatgccgaacacaagctgcagttttgggccgtgacggcagaaaatgagccctctgctggcctgctttctggttaccctttccagtgccttggtttcacaccggagcaccagcgggatttcatcgctagagatctgggacccacgctggccaactccacccatcacaacgtgcgcctcttgatgctggatgaccagagactcctcctcccacactgggctaaggttgttcttactgatccagaagccgccaaatacgttcacggaattgccgtccactggtatttggacttcctcgcccccgctaaagctacactgggcgaaacgcacagactgtttcccaacaccatgctctttgctagtgaagcgtgcgtcggttctaagttctgggaacagagcgtacgcctgggctcatgggacaggggaatgcaatattcccacagcataatcaccaacctgctgtatcatgtggtcgggtggacagactggaacttggccttgaaccccgagggcggccccaactgggtgagaaactttgtggacagtccaattatcgtcgatattactaaggatacattctataagcaacctatgttctatcacctcggccatttctctaagtttatccctgaaggcagccagcgagtcggactggtggcaagtcaaaagaacgatctcgatgcggtggcattgatgcaccccgacggaagtgcagttgttgtggtcctgaataggagctcaaaggatgttccactgaccataaaggaccctgccgtgggcttcttggaaaccatttccccagggtacagcattcacacctacctgtggcgacggcagtga

SEQ ID NO: 17 - DNA, 1611 bp, synthetic construct
  misc_feature 1..1611: note = CDS
  source 1..1611: mol_type = other DNA
atggagtttagctcccctagcagagaggagtgccctaagcccctgagtagagtgagcattatggccggctccctgacagggctgctccttctgcaggccgtgagctgggccagcggggcccggccctgcatccctaaatccttcgggtactcctcggtggtgtgtgtgtgcaatgctacctactgtgattcctttgaccccccaacatttcccgccctgggcacctttagcaggtacgagtccacacggtccgggaggaggatggagctgagcatgggccccattcaggccaaccacaccggcaccggcctcctgctgactctgcagcccgagcagaagttccagaaggtgaagggctttggcggcgccatgactgacgccgccgccctgaacatcctggccctgagcccccccgcccaaaacctgctgctgaagtcctactttagtgaggaggggatcggctacaacattattcgggtgcctatggccagctgcgacttctccatccggacatacacctacgctgatacacccgatgacttccagctccacaactttagcctgcctgaggaggatactaagctgaagattcccctgatccacagggccctccagcttgcccagagacccgtgagcctcctggccagcccctggacaagccccacctggctcaagaccaacggggccgtgaacgggaaggggtccctgaaggggcagccaggcgacatctaccaccagacctgggcccggtacttcgtgaagttcctggatgcctacgccgagcacaagctgcagttttgggccgtgaccgccgagaacgagcccagcgccggcctgctgagcgggtaccccttccagtgcctggggtttacaccagagcaccagcgggactttattgctcgggacctcggccccaccctggccaacagcacacaccacaacgtgaggctgctgatgctggatgatcagcggctcctgctgccccactgggctaaggtggtcctgactgaccctgaggccgccaagtacgtgcacggcatcgccgtgcattggtacctggatttcctggcccccgccaaggccaccctgggggagacacaccggctgttccctaacacaatgctgtttgccagcgaggcctgcgtgggctccaagttctgggagcagagcgtgcggctggggagctgggatcgggggatgcagtacagccacagcattatcaccaacctgctctaccacgtggtggggtggaccgactggaacctggccctgaaccccgagggcggccccaactgggtcaggaacttcgtcgatagccccataatcgtggatatcacaaaggataccttttacaagcagcccatgttctaccacctcgggcacttctccaagttcatccccgagggctcccagcgggtgggcctggtcgccagccagaagaacgatctggacgccgtggccctgatgcaccctgacgggagcgccgtcgtggtggtgctgaaccggtccagcaaggacgtccccctgacaatcaaggaccccgccgtcgggttcctggagaccatcagcccaggctacagcattcacacatacctgtggcggcggcagtga

SEQ ID NO: 18 - DNA, 1611 bp, synthetic construct
  misc_feature 1..1611: note = CDS
  source 1..1611: mol_type = other DNA
atggagttcagctctccatctagggaggagtgccctaagccactgtctcgtgtgtccatcatggccggcagcctgacaggcctcctgctgctgcaagccgtgtcttgggcctccggcgccagaccttgcatccctaagtctttcggctactctagcgtggtgtgcgtgtgcaacgccacatactgcgactctttcgacccaccaaccttccccgccctcggcaccttctcccggtacgagtctacacggtccggcaggcggatggagctgtcgatgggcccaattcaggccaaccacacaggcacaggcctgctgctcacactccaacccgagcagaagttccagaaggtgaagggcttcggcggcgccatgaccgacgccgccgccctcaacattctcgccctgtccccacccgcccagaacctgctcctgaagtcttacttcagcgaggagggcattggctacaacattatccgggtgcctatggcctcttgcgacttctctattagaacatacacatacgccgacacccccgacgacttccagctgcacaacttcagcctcccagaggaggacacaaagctcaagattccactgattcaccgcgccttgcaactcgcccagagaccagtgtctctgctcgcctccccttggacatctcctacatggctcaagacaaacggcgccgtgaacggcaagggctctctgaagggccagccaggcgacatttaccaccagacatgggcccggtacttcgtgaagttcctcgacgcctacgccgagcacaagttacaattctgggccgtgacagccgagaacgagccatctgccggcctcctgtctggctaccctttccagtgcctcggcttcacccccgagcaccagcgggacttcattgcacgcgacctcggcccaacactcgccaactctacccaccacaacgtgagactgctgatgctcgacgaccagagactgctgctccctcactgggccaaggtggtgctgaccgaccctgaggccgccaagtacgtgcacggcattgccgtgcactggtatctggacttcctggccccagccaaggccacactcggcgagacccacagactgttcccaaacaccatgctgttcgccagcgaggcctgcgtgggcagcaagttctgggagcagtctgtgagactcggctcttgggaccgcggaatgcagtactctcactctatcattacaaacctcctgtaccacgtggtgggctggaccgactggaacctcgccctcaaccccgagggcggccctaactgggtgcggaacttcgtggactctcctattattgtggacattacaaaggacacattctacaagcagcctatgttctaccacctcggccacttcagcaagttcatccctgagggctctcagagagtgggcctggtggcctctcagaagaacgacctggacgccgtggccctgatgcaccccgacggcagcgccgtggtggtggtgctgaacaggtcgagcaaggacgtgccactgacaattaaggaccccgccgtgggcttccttgagacaatttctccaggctactctattcacacatacctgtggcggagacagtga

SEQ ID NO: 19 - DNA, 1611 bp, synthetic construct
  misc_feature 1..1611: note = CDS
  source 1..1611: mol_type = other DNA
atggagttcagcagccccagcagggaagagtgccccaagcccctgagcagggtgagcatcatggccggaagcctgaccgggctgctgctgctgcaggccgtgagctgggccagcggagctaggccttgcatccccaaaagcttcggctacagctccgtggtgtgcgtgtgtaacgccacctattgtgactctttcgacccccccacctttcccgccctgggcactttcagcagatatgagtccacccgcagcggcagacggatggagctgagcatgggacccattcaggccaaccacaccggcaccggcctgctgctgaccctgcagcctgaacagaaattccagaaagtgaagggcttcggcggcgccatgaccgacgccgctgctctgaacatcctggccctgtccccccccgcccagaacctgctgctgaagagctacttcagcgaggaaggcatcggctacaatatcatccgcgtgcctatggccagctgcgacttttctatcaggacctacacctacgccgacacccccgacgactttcagctgcacaacttcagcctgccagaggaagacaccaaactgaagatccccctgatccacagagccctgcagctggcccagagacccgtgagcctgctggccagcccctggacatcccccacctggctgaagaccaacggcgccgtgaacggcaaaggcagcctgaaaggccagcccggagatatttaccaccagacctgggccagatactttgtgaaattcctggacgcctacgccgaacacaagctgcagttctgggccgtgacagccgagaatgagcccagcgctggcctgctgagcggctaccctttccagtgtctgggcttcacccccgagcaccagagagatttcatcgcccgcgacctgggccccaccctggctaatagcacccaccacaacgtgagactgctgatgctggacgaccagaggctgctgctgccccactgggctaaagtggtgctgaccgaccccgaagccgccaaatacgtgcacggcatcgccgtgcactggtacctggatttcctggcccccgccaaggccaccctgggagaaacccacagactgtttcccaataccatgctgttcgccagcgaagcctgcgtgggcagcaagttctgggagcagagcgtgcgcctgggcagctgggacagaggaatgcagtacagccacagcatcatcaccaacctgctgtaccacgtggtgggatggacagattggaacctggccctgaaccccgagggcggacccaactgggtgagaaactttgtggacagccccatcatcgtggacatcaccaaggacaccttctacaaacagcctatgttctaccacctgggccacttctccaagttcattcccgagggcagccagagagtgggcctggtggccagccagaagaacgatctggacgccgtggccctgatgcaccccgacggaagcgccgtggtggtggtgctgaacagaagcagtaaggatgtgcccctgaccatcaaggatcccgctgtgggattcctggaaaccatcagccctggctactccatccacacctacctgtggagaagacagtga

SEQ ID NO: 20 - DNA, 1611 bp, synthetic construct
  misc_feature 1..1611: note = CDS
  source 1..1611: mol_type = other DNA
atggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgatatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgccagagatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccggtccagcaaagatgtgcccctgaccatcaaggatcccgccgtgggattcctggaaacaatcagccctggctactccatccacacctacctgtggcggagacagtga

SEQ ID NO: 21 - DNA, 1611 bp, synthetic construct
  misc_feature 1..1611: note = CDS
  source 1..1611: mol_type = other DNA
atggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtga

SEQ ID NO: 22 - DNA, 1611 bp, Homo sapiens
  source 1..1611: mol_type = genomic DNA
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 23 - DNA, 564 bp, synthetic construct
  misc_feature 1..564: note = CDS
  source 1..564: mol_type = other DNA
atgtggtgcgcgagcccagttgctgtggtggccttttgcgccgggcttttggtctctcacccggtgctgacgcaaggccaggaggccggcggaagacctggtgctgattgcgaggtgtgcaaggaattcctgaaccggttttataagtccctgatcgacagaggcgtgaacttcagcctggataccatcgagaaagaactcatctccttctgcctggacaccaagggcaaggaaaaccggctgtgctactacctgggcgccacaaaggacgccgctacaaagatcctgagcgaggtgaccagaccaatgagcgtgcacatgcctgccatgaaaatctgtgaaaaactgaaaaagctggacagccagatctgcgagctgaagtacgagaagaccctggatctggcttctgtcgacctgagaaagatgcgggtggccgaactgaagcagatcctgcacagctggggcgaggaatgtagagcctgcgccgagaagaccgactacgtgaatctgattcaggagctggcccctaagtacgccgccacccaccccaagacagagctgtga

SEQ ID NO: 24 - DNA, 564 bp, synthetic construct
  misc_feature 1..564: note = CDS
  source 1..564: mol_type = other DNA
atgtggtgcgcgagcccagttgctgtggtggccttttgcgccgggcttttggtctctcacccggtgctgacgcagggccaggaggccggggggcggcccggcgccgactgcgaggtgtgtaaggagtttctgaaccggttctacaagtccctgatcgatcggggcgtcaacttcagcctggatactatcgagaaggagctgatcagcttctgcctggatacaaaggggaaggagaaccggctgtgctactacctgggggccaccaaggacgccgccaccaagatcctgtccgaggtgacccggcccatgagcgtgcacatgcccgccatgaagatctgcgagaagctgaagaagctggatagccagatctgcgagctgaagtacgagaaaacactggacctggccagcgtggacctgaggaagatgagggtggccgagctgaagcagattctgcacagctggggagaggaatgcagggcctgcgctgaaaagactgattacgtcaacctgatccaggagctggccccaaagtacgctgccacacaccccaagacagagctgtga

SEQ ID NO: 25 - DNA, 564 bp, synthetic construct
  misc_feature 1..564: note = CDS
  source 1..564: mol_type = other DNA
atgtggtgcgcgagcccagttgctgtggtggccttttgcgccgggcttttggtctctcacccggtgctgacgcagggccaggaggctggaggcagacctggcgctgactgcgaggtgtgcaaggagttcctgaacaggttctacaagagcctgatcgacaggggagtgaacttcagcctggacaccattgagaaggagctgatcagcttctgcctggacaccaagggcaaggagaacaggctgtgctactacctgggagccaccaaggacgctgccaccaagatcctgtctgaggtgaccagacccatgagtgtgcatatgcctgccatgaagatctgtgagaagctgaagaagctggacagccagatctgtgagctgaagtatgagaagaccctggacctggcctctgtggacctgaggaagatgagagtggccgagctgaagcagatcctgcacagctggggagaggagtgcagagcctgcgccgagaagaccgactacgtgaacctgatccaggagctggctcctaagtacgccgccacccatcccaagaccgagctgtga

SEQ ID NO: 26 - DNA, 564 bp, Homo sapiens
  source 1..564: mol_type = genomic DNA
atgtggtgcgcgagcccagttgctgtggtggccttttgcgccgggcttttggtctctcacccggtgctgacgcagggccaggaggccggggggcggccaggggccgactgtgaagtatgtaaagaattcttgaaccgattctacaagtcactgatagacagaggagttaacttttcgctggacactatagagaaagaattgatcagtttttgcttggacaccaaaggaaaagaaaaccgcctgtgctattatctaggagccacaaaagacgcagccacaaagatcctaagtgaagtcactcgcccaatgagtgtgcatatgcctgcaatgaagatttgtgagaagctgaagaagttggatagccagatctgtgagctgaaatatgaaaaaacactggacttggcatcagttgacctgcggaagatgagagtggcagagctgaagcagatcctgcatagctggggggaggagtgcagggcctgtgcagaaaaaactgactatgtgaatctcattcaagagctggcccccaagtatgcagcgacacaccccaaaacagagctctga

SEQ ID NO: 27 - DNA, 636 bp, synthetic construct
  misc_feature 1..636: note = CDS
  source 1..636: mol_type = other DNA
atgaagttatgggatgtcgtggctgtctgcctggtgctgctccacaccgcgtccgcctttccactgcctgccggcaagcggccccccgaggcccctgctgaagatagaagcctgggcagacggcgggcccctttcgccctgtctagcgacagcaacatgcctgaggactaccccgatcagttcgacgacgtgatggacttcatccaggctacaatcaagagactgaagcggagccctgacaaacagatggccgtgctgccaagacgcgagagaaatagacaggccgccgctgctaatcctgagaacagcagaggcaaaggcagaaggggccagagaggcaagaacagaggatgtgtgctgaccgccattcacctgaacgtgacagacctgggactgggatatgagaccaaggaagagctgatcttcagatactgcagcggctcttgcgacgccgccgaaaccacctacgacaagatcctgaaaaacctctccagaaaccggcggctggtgtccgataaggtgggccaagcctgctgcagacctatcgcctttgacgatgatctgagcttcctggatgacaacctggtctaccacatcctgagaaagcacagcgccaagcggtgcggctgtatctga

SEQ ID NO: 28 - DNA, 636 bp, synthetic construct
  misc_feature 1..636: note = CDS
  source 1..636: mol_type = other DNA
atgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaagcggtctcccgataagcagatggccgtgttgccccggcgggagcggaacaggcaggccgccgccgccaaccccgagaacagcagggggaagggcaggcgggggcagcggggcaagaacaggggctgcgtgctgaccgccatccacctgaacgtgacagacctggggctggggtacgagacaaaggaggagctgatcttcaggtactgcagcgggagctgcgacgctgccgagaccacatacgataagatcctgaagaacctgagcaggaacaggaggctggtgagcgacaaggtggggcaggcctgttgcaggcccattgcctttgatgatgatctgagctttcttgatgataacctggtgtaccatattctgagaaagcactccgccaagagatgcggatgcatttga

SEQ ID NO: 29 - DNA, 636 bp, synthetic construct
  misc_feature 1..636: note = CDS
  source 1..636: mol_type = other DNA
atgaagttatgggatgtcgtggctgtctgcctggtgctgctccacaccgcgtccgccttccctctgcctgctggcaagaggcctcccgaggctcctgccgaggacagaagcctcggcagaaggagagctccttttgccctgagcagcgacagcaacatgcctgaggactaccccgaccagtttgatgacgtgatggacttcatccaggccaccatcaaaagactgaaaaggtcacctgacaagcagatggctgtgctgcctagaagggagaggaacaggcaggctgccgctgccaatcctgagaatagcagaggcaagggcaggagaggacagagaggcaagaacagaggctgtgtgctgacagccatccacctgaatgtgacagacctgggactgggctatgagaccaaagaggagctgatctttagatactgctctggcagctgtgatgccgctgagaccacctacgacaagatcctgaagaacctgagcagaaacaggagactggtgagcgacaaggtgggccaggcctgctgcaggcccatcgccttcgacgacgacctgagcttcctggacgacaacctggtgtaccacatcctgagaaagcacagcgccaagaggtgcggctgcatctga

SEQ ID NO: 30 - DNA, 636 bp, Homo sapiens
  source 1..636: mol_type = genomic DNA
atgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga

SEQ ID NO: 31 - protein, 480 aa, Homo sapiens
MNASEFRRRGKEMVDYMANYMEGIEGRQVYPDVEPGYLRPLIPAAAPQEPDTFEDIINDVEKIIMPGVTHWHSPYFFAYFPTASSYPAMLADMLCGAIGCIGFSWAASPACTELETVMMDWLGKMLELPKAFLNEKAGEGGGVIQGSASEATLVALLAARTKVIHRLQAASPELTQAAIMEKLVAYSSDQAHSSVERAGLIGGVKLKAIPSDGNFAMRASALQEALERDKAAGLIPFFMVATLGTTTCCSFDNLLEVGPICNKEDIWLHVDAAYAGSAFICPEFRHLLNGVEFADSFNFNPHKWLLVNFDCSAMWVKKRTDLTGAFRLDPTYLKHSHQDSGLITDYRHWQIPLGRRFRSLKMWFVFRMYGVKGLQAYIRKHVQLSHEFESLVRQDPRFEICVEVILGLVCFRLKGSNKVNEALLQRINSAKKIHLVPCHLRDKFVLRFAICSRTVESAHVQRAWEHIKELAADVLRAERE

SEQ ID NO: 32 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 33 - protein, 187 aa, Homo sapiens
MWCASPVAVVAFCAGLLVSHPVLTQGQEAGGRPGADCEVCKEFLNRFYKSLIDRGVNFSLDTIEKELISFCLDTKGKENRLCYYLGATKDAATKILSEVTRPMSVHMPAMKICEKLKKLDSQICELKYEKTLDLASVDLRKMRVAELKQILHSWGEECRACAEKTDYVNLIQELAPKYAATHPKTEL

SEQ ID NO: 34 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 35 - DNA, 69 bp, synthetic construct
  misc_feature 1..69: note = CDS
  source 1..69: mol_type = other DNA
ggaagcggacagtgtactaattatgctctcttgaaattggctggagatgttgagagcaaccctggacct

SEQ ID NO: 36 - protein, 23 aa, equine rhinitis a virus
GSGQCTNYALLKLAGDVESNPGP

SEQ ID NO: 37 - DNA, 75 bp, synthetic construct
  misc_feature 1..75: note = CDS
  source 1..75: mol_type = other DNA
ggaagcggagtgaaacagactttgaattttgaccttctcaagttggcgggagacgtggagtccaaccctggacct

SEQ ID NO: 38 - protein, 25 aa, foot-and-mouth disease virus
GSGVKQTLNFDLLKLAGDVESNPGP

SEQ ID NO: 39 - DNA, 63 bp, synthetic construct
  misc_feature 1..63: note = CDS
  source 1..63: mol_type = other DNA
ggaagcggagagggcagaggaagtctgctaacatgcggtgacgtcgaggagaatcctggacct

SEQ ID NO: 40 - protein, 21 aa, thoseaasigna virus
GSGEGRGSLLTCGDVEENPGP

SEQ ID NO: 41 - DNA, 66 bp, synthetic construct
  misc_feature 1..66: note = CDS
  source 1..66: mol_type = other DNA
ggaagcggagctactaacttcagcctgctgaagcaggctggagacgtggaggagaaccctggacct

SEQ ID NO: 42 - protein, 22 aa, porcine teschovirus-1
GSGATNFSLLKQAGDVEENPGP

SEQ ID NO: 43 - DNA, 22 bp, synthetic construct
  misc_feature 1..22: note = CDS
  source 1..22: mol_type = other DNA
tttataatttcttcttccagaa

SEQ ID NO: 44 - DNA, 552 bp, synthetic construct
  misc_feature 1..552: note = CDS
  source 1..552: mol_type = other DNA
acgttactggccgaagcgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataata

SEQ ID NO: 45 - DNA, 1611 bp, synthetic construct
  misc_feature 1..1611: note = CDS
  source 1..1611: mol_type = other DNA
atggagttcagctctcccagcagagaagaatgtcctaagcctctgagcagggtgtccatcatggctggcagcctgacaggactcctgctcctgcaggctgtgagctgggcctctggagccaggccttgcattcccaagagcttcggctacagcagcgtggtgtgcgtgtgcaacgccacctactgcgacagctttgaccctcctacctttcctgccctgggaacattcagcagatatgagagcaccaggagcggccggagaatggagctgagcatgggacccatccaggccaaccacacaggaacaggcctcctgctcaccctgcagcctgagcagaagtttcagaaggtgaagggcttcggaggagccatgacagatgctgccgctctgaacatcctggccctgagccctcctgcccagaacctcctgctcaagagctacttcagcgaagaaggaatcggctacaacatcatcagagtgcccatggccagctgcgactttagcattaggacatatacctacgccgatacacctgatgacttccagctgcataactttagcctgcctgaggaggataccaagctgaaaatccctctgatccatcgcgccctgcagctggcccagagacctgtgtccctgctggccagcccttggacaagccctacatggctgaagaccaatggcgctgtgaatggcaaaggcagcctgaagggccagcctggagatatctatcaccagacctgggccaggtactttgtgaagttcctggatgcctatgccgagcacaagctgcagttctgggctgtgaccgctgagaatgaaccctctgccggactgttgagcggctatccctttcagtgtctgggattcacccctgagcaccagagggacttcattgcccgcgacctgggccctaccctggccaacagcactcaccacaatgtgcgcctgctgatgctggacgatcagagactgctgctgccccactgggccaaggtggtgctgaccgaccctgaggccgccaagtacgtgcacggcatcgccgtgcactggtacctggacttcctggctcctgccaaggccaccctgggcgagacccacaggctgttccctaacaccatgctgttcgccagcgaggcctgcgtgggctccaagttctgggagcagtccgtgaggctgggaagctgggacaggggcatgcagtacagccacagcatcatcaccaacctgctgtaccacgtggtgggctggaccgactggaacctggccctgaaccctgagggcggcccaaactgggtgcgcaacttcgtggacagccctatcatcgtggacatcaccaaggacaccttctacaagcagcccatgttctaccacctgggccacttcagcaagtttattcctgagggcagccaaagggtgggactggtggccagccagaaaaatgacctggacgccgtggctctgatgcatcctgatggcagcgccgtcgtggttgtcctgaaccgcagcagcaaggacgtgcccctgaccatcaaggatcctgctgtgggcttcctggagaccatcagccctggctacagcatccacacctacctgtggaggagacagtga

SEQ ID NO: 46 - DNA, 1443 bp, synthetic construct
  misc_feature 1..1443: note = CDS
  source 1..1443: mol_type = other DNA
atgaatgccagcgagtttagaagaaggggcaaagagatggttgactacatggccaattacatggaaggcatcgagggcaggcaggtgtaccctgatgtggaacctggatatctgagacctctgattcctgctgcagctcctcaggagcctgacacattcgaggatatcatcaacgacgtggagaaaatcatcatgcctggcgtcacccactggcactctccttacttctttgcctatttccctacagccagcagctaccctgccatgctggctgacatgctgtgtggagccatcggctgcatcggcttcagctgggcggcaagcccagcctgcaccgagctggaaacagtgatgatggactggctgggaaagatgctggaactgcctaaggcctttctgaatgagaaggctggcgagggaggaggagtgatccagggctctgcttctgaggccaccctggttgctctgcttgctgccagaaccaaggtgatccacagactgcaggctgcctctcctgagctgacccaggcagccatcatggaaaagctggtggcttacagctctgaccaggcccattccagcgtggaaagggccggcctgattggcggagtgaagctcaaggctatccctagcgatggcaactttgccatgagagcctctgccctgcaagaggccctggaaagagataaggccgctggactcatcccgttcttcatggtggccacactgggcacaacaacatgctgcagctttgacaacctgctggaagtgggccctatctgcaacaaagaggacatctggctgcatgtggatgctgcctacgctggctctgccttcatctgtcctgagttcagacatctgctgaacggcgtggagtttgctgatagcttcaacttcaaccctcacaagtggctgctggtcaactttgactgctctgctatgtgggtgaagaagagaaccgacctgacaggagctttcagactggatcccacctacctgaaacacagccaccaggattctggcctgatcaccgattacagacactggcagattcctctgggcagaaggttcagaagcctgaagatgtggtttgtgttcagaatgtatggcgtgaagggcctgcaagcctacatcagaaagcatgtgcagctgagccatgagttcgagagcctggtgaggcaggaccctagatttgagatctgtgtggaggtgatcctgggcctggtgtgcttcaggctgaagggcagcaacaaggtgaacgaggctctgctgcagaggatcaacagcgccaagaagatccacctggtgccttgccacctgagagacaagtttgtgctgagatttgctatttgtagcagaaccgtggaaagcgcccacgtgcagagagcctgggagcacatcaaggaactggccgctgatgtgctgagagccgagcgggaatga

SEQ ID NO: 47 - DNA, 1611 bp, synthetic construct
  misc_feature 1..1611: note = CDS
  source 1..1611: mol_type = other DNA
atggagttttcaagtccttccagagaggaatgtcccaagcctttgagtagggtaagcatcatggctggcagcctcacaggattgcttctacttcaggcagtgtcgtgggcatcaggtgcccgcccctgcattcccaagagctttggctacagcagcgtggtgtgcgtgtgcaacgccacctactgtgacagctttgaccctcctacctttcctgctctgggaacattcagcagatatgagagcaccaggtctggccggagaatggagctgagcatgggacccatccaggccaaccacacaggaacaggactcctgctcaccctgcagcctgagcagaagtttcagaaggtgaagggcttcggaggagccatgacagatgctgccgctctgaacatcctggccctgagccctcctgcccagaacctcctgctcaagagctacttcagcgaagaaggaatcggctacaacatcatcagagtgcccatggccagctgcgactttagcattaggacatatacctacgccgatacacctgatgacttccagctgcataactttagcctgcctgaggaggataccaagctgaaaatccctctgatccatcgcgccctgcagctggcccagagacctgtgtccctgctggccagcccttggacaagccctacatggctgaagaccaatggcgctgtgaatggcaaaggcagcctgaagggccagcctggagatatctatcaccagacctgggccaggtactttgtgaagttcctggatgcctatgccgagcacaagctgcagttctgggctgtgaccgctgagaatgaaccctctgctggactgttgtctggctatccctttcagtgtctgggattcacacctgagcaccagagggacttcattgccagagacctgggacctaccctggccaacagcactcaccacaatgtgagactgctgatgctggatgatcagagactgctgctgcctcattgggccaaggtggtgctgacagatcctgaggcagccaagtatgtgcacggcattgccgtgcactggtacctggacttcctggctcctgccaaggccaccctgggagagacccacaggctgttccctaacaccatgctgttcgcctctgaagcctgcgtgggctccaagttctgggagcagagcgtgaggctgggaagctgggacaggggcatgcagtacagccacagcatcatcaccaacctgctgtaccatgtggtgggctggaccgactggaacctggccctgaaccctgagggaggcccaaactgggtgcgcaacttcgtggacagccctatcattgtggacatcaccaaggacaccttctacaagcagcccatgttctaccacctgggccacttcagcaagtttattcctgagggcagccaaagggtgggactggtggccagccagaaaaatgacctggacgccgtggctctgatgcatcctgatggctctgctgtggtggttgtcctgaacagaagcagcaaggatgtgccactgaccatcaaggatcctgctgtgggcttcctggagaccatcagccctggctacagcatccacacctacctgtggaggagacagtga

SEQ ID NO: 48 - DNA, 3103 bp, synthetic construct
  misc_feature 1..3103: note = CDS
  source 1..3103: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatccaccatgaacgcaagtgaattccgaaggagagggaaggagatggtggattacgtggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagtaggagtgaagccaggacctgcagaagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 49 - DNA, 3597 bp, synthetic construct
  misc_feature 1..3597: note = CDS
  source 1..3597: mol_type = other DNA
accggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgact

SEQ ID NO: 50 - DNA, 630 bp, synthetic construct
  misc_feature 1..630: note = CDS
  source 1..630: mol_type = other DNA
tctagagtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagc

SEQ ID NO: 51 - DNA, 580 bp, synthetic construct
  misc_feature 1..580: note = CDS
  source 1..580: mol_type = other DNA
tctagagtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactgg

SEQ ID NO: 52 - DNA, 530 bp, synthetic construct
  misc_feature 1..530: note = CDS
  source 1..530: mol_type = other DNA
tctagagtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctt

SEQ ID NO: 53 - DNA, 480 bp, synthetic construct
  misc_feature 1..480: note = CDS
  source 1..480: mol_type = other DNA
tctagagtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctt

SEQ ID NO: 54 - DNA, 430 bp, synthetic construct
  misc_feature 1..430: note = CDS
  source 1..430: mol_type = other DNA
tctagagtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctg

SEQ ID NO: 55 - DNA, 380 bp, synthetic construct
  misc_feature 1..380: note = CDS
  source 1..380: mol_type = other DNA
tctagagtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgct

SEQ ID NO: 56 - DNA, 330 bp, synthetic construct
  misc_feature 1..330: note = CDS
  source 1..330: mol_type = other DNA
tctagagtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttg

SEQ ID NO: 57 - DNA, 1184 bp, synthetic construct
  misc_feature 1..1184: note = CDS
  source 1..1184: mol_type = other DNA
tctagagtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaacgcgt

SEQ ID NO: 58 - DNA, 1718 bp, synthetic construct
  misc_feature 1..1718: note = CDS
  source 1..1718: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcgggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagcgcctctgctaaccatgttcatgccttcttctttttcctacagctcctgggcaacgtgctggttattgtgctgtctcatcattttggcaaa

SEQ ID NO: 59 - DNA, 1366 bp, synthetic construct
  misc_feature 1..1366: note = CDS
  source 1..1366: mol_type = other DNA
aagctttgagagaaaagggaccagatcttattcctcaccgtggctttaacacttagagaaaatgcatcccctctaatcaataagtcatcgacagtgggtagatggaggaacggcagtgcgtagtaggatgcgtgctaagcatagtctcgtgcatgggtgcatagatcgctgggcaggtggacaaggtgggggtggataaagaagtgggtagatgattgatgttaggtaaatatcactgggtggacagatgggtggtaggtggatggatggttagaatagtcagaagagggatggattgataaggtgaacagatgataaatgggtgatagactggaagggttgtcaaaagaggataagggaagtgtgagctagccgtatttctaaggtcagtaatagagttgggagaagaggttaagttacatccatttaaacctcacacgaagctgagtgggaatggacttgctgccgttggtgaggaaagcgttgcatttcccgtgtgcttggttgtggaagtgctcaggtcccacatgaagcagtcaggttactgcggcttacagaggagccagatccaaatgccccgagtaagcacgtccccgagccagaggcctccagcggaatccgggagagggattgctcagtgccctgcttccctggactgtaagctgcagaaagatgtgggaagtcctgttctccactgagaacactaaaagcaccttttgtcaaacgaccgcttcacatctggggcttgtgcactggtggccttttaaaccagagacaacccacaagatacctaacctgcggggctctctggtacagtgagcaactcaggaaatgctttggcttgattgctgtgggctctcaggccatcgccctctggagtggttcttttaatgagaacctgaagattggcccctgagccatgtataccaagcaagctcaatccaggttagctccctctggttggggcaagctaacgtgctccttgggccccgcgcgtaactgtgcgttttataggagacagctagttcaagaccccaggaagaaagcggctttgtccccctctaggcctcgtacaggcccacattcatatctcattgttgttgcaggggaggcagatgcgatccagaacaatgggacctcggctgaggacacggcggtgacagactccaagcacacagcagacccaaagaataactggcaaggcgcccacccagctgacccagggaaccgcccccacttgatccgcctcttttcccgagatgccccgggaagggaggacaacaccttcaaagacaggccctcagagtccgacgagcttcagaccatccaagaagaccccacagcagcttccggaggcctggatgtg

SEQ ID NO: 60 - DNA, 812 bp, synthetic construct
  misc_feature 1..812: note = CDS
  source 1..812: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

(19) United States
(12) Patent Application Publication
(10) Pub. No.: US 2025/0041456 A1
(43) Pub. Date: Feb. 6, 2025

(54) RECOMBINANT AAV VECTORS FOR TREATING NEURODEGENERATIVE DISORDERS

(71) Applicant: SHANGHAI VITALGEN BIOPHARMA CO., LTD., Shanghai (CN)

(72) Inventors: Yezheng TAO, Shanghai (CN); Shin-Shay TIAN, Shanghai (CN); Xiaoping ZHAO, Shanghai (CN)

(73) Assignee: SHANGHAI VITALGEN BIOPHARMA CO., LTD., Shanghai (CN)

(21) Appl. No.: 18/919,740

(22) Filed: Oct. 18, 2024

Related U.S. Application Data
(63) Continuation of application No. PCT/CN2023/089291, filed on Apr. 19, 2023.

(30) Foreign Application Priority Data
Apr. 19, 2022 (WO) PCT/CN2022/087771

(51) Int. Cl. A61K 48/00; C07K 14/52; C12N 9/24; C12N 9/88; C12N 15/86

(52) U.S. Cl. CPC A61K 48/0058; C07K 14/52; C12N 9/2402; C12N 9/88; C12N 15/86; C12Y 302/01045; C12Y 401/01028; C12N 2750/14143

(57) ABSTRACT

Provided is a recombinant adeno-associated viral (rAAV) vector comprising one or two of (a) to (c): (a) a nucleotide sequence encoding aromatic L-amino acid decarboxylase (AADC), (b) a nucleotide sequence encoding glucocerebrosidase (GBA1); and (c) a nucleotide sequence encoding a neurotrophic factor (NTF), such as cerebral dopamine neurotrophic factor (CDNF) or glial cell derived neurotrophic factor (GDNF), for treating neurodegenerative disorders, particularly Parkinson's disease (PD), Multiple system atrophy (MSA), Gaucher's disease (GD), and other proteinopathies. Also provided herein are viral particles comprising the rAAV vector, a pharmaceutical composition comprising the viral particles, and uses thereof.

### CROSS-REFERENCE TO RELATED APPLICATION

The present application is a Continuation application of PCT International Application No. PCT/CN2023/089291 filed on Apr. 19, 2023, which claims priority to Chinese Patent Application No. PCT/CN2022/087771, filed Apr. 19, 2022, the disclosure of which all is incorporated herein by reference in its entirety.

### REFERENCE TO SEQUENCE LISTING

In accordance with 37 CFR § 1.52(e)(5) and with 37 CFR § 1.831, the specification makes reference to a Sequence Listing submitted electronically as a .xml file named 19684.0088FPWO Sequence Listing.xml copy, created and filed herewith is 128,000 bytes in size. The entire contents of the Sequence Listing are hereby incorporated by reference.

### FIELD OF THE INVENTION

The present disclosure relates to the technical field of gene therapy. Specifically, the present disclosure provides a recombinant adeno-associated viral (rAAV) vector comprising one or two of (a) to (c): (a) a nucleotide sequence encoding aromatic L-amino acid decarboxylase (AADC), (b) a nucleotide sequence encoding glucocerebrosidase (GBA1); and (c) a nucleotide sequence encoding a neurotrophic factor (NTF), such as cerebral dopamine neurotrophic factor (CDNF) or glial cell derived neurotrophic factor (GDNF), for treating neurodegenerative disorders, particularly Parkinson's disease (PD), Multiple system atrophy (MSA), Gaucher's disease (GD), AADC deficiency (AADCD), and other proteinopathies. Also provided herein are viral particles comprising the rAAV vector, a pharmaceutical composition comprising the viral particles, and uses thereof.

### BACKGROUND

Neurodegenerative diseases (NDs) occur when nerve cells in the brain or peripheral nervous system progressively lose function and ultimately die, resulting in loss of mobility, coordination, strength, sensation, and cognition. Due to their slow and progressive nature and lack of suitable treatments to slow the disease progression or cure, NDs are associated with large socioeconomic and personal costs and sufferings. Aging is the primary risk factor for most NDs. With people living longer, it means more people will be afflicted with NDs in the coming decades to place huge financial burden on our society and bring immense global public health challenges.

Parkinson's disease (PD), caused by the degeneration of midbrain dopaminergic transmission and characterized by both motor symptoms (e.g., tremor and rigidity) and non-motor symptoms (e.g., mental retardation and depression), affects almost 0.3% of the general population and ˜1% of the population over the age of 60 (de Lau, L. M. and M. M. Breteler, Epidemiology of Parkinson's disease. Lancet Neurol, 2006. 5(6): p. 525-35).

Most PD cases are sporadic and caused by unknown factors. Only a minority of cases has family history. Currently, six specific human genome loci have been identified to contain genes whose mutations may lead to the onset of rare familial forms of PD (Klein, C. and A. Westenberger, Genetics of Parkinson's disease. Cold Spring Harb Perspect Med, 2012. 2(1): p. a008888). It is widely accepted that the etiology of PD results from an elaborate interplay of intrinsic factors like genetics with extrinsic environmental factors. The pathological changes and their relation to PD symptoms are well-established (Kouli, A., K. M. Torsney, and W. L. Kuan, Parkinson's Disease: Etiology, Neuropathology, and Pathogenesis, in Parkinson's Disease: Pathogenesis and Clinical Aspects, T. B. Stoker and J. C. Greenland, Editors. 2018: Brisbane (AU)). PD is pathologically characterized by the degeneration and loss of dopaminergic neurons in the substantia nigra pars compacta (SNc) and the loss of dopaminergic pathway from SNc to striatum (Putamen and caudate nuclei), leading to a reduced levels of dopamine in the striatum that results in impaired motor function. Based on this knowledge, several drugs have been developed by different approaches to restore the dopamine levels.

Another pathological hallmark of PD is the formation of Lewy bodies (LBs) in patient's brain, or the so-called “Lewy pathology”. The alpha-synuclein (α-syn)-containing LBs or Lewy neurites induce cellular toxicity within DA neurons and other neuron subtypes, and is one of the main PD pathogenesis (Teil, M., et al., Targeting alpha-synuclein for PD Therapeutics: A Pursuit on All Fronts. Biomolecules, 2020. 10(3)). Multiple strategies targeting the Lewy pathology are currently under investigation, including stabilization of the α-syn physiological conformation, decrease of α-syn expression, inhibition of α-syn aggregation, and increase of α-syn clearance. Moreover, Lewy pathology is not restricted to the dopaminergic circuitry but extends to other brain regions via cell-to-cell propagation of α-syn aggregates, which might be one of the mechanisms underlying the non-motor PD symptoms. So theoretically, therapeutic treatments tackling α-syn aggregation may benefit both motor and non-motor functions of the PD patients.

Among the currently available treatments, L-DOPA (levodopa) is effective at treating motor symptoms of some of the PD patients. But L-DOPA has limited effects on treating non-motor symptoms or late stage PD patients. Moreover, it was shown that L-DOPA could cause severe side effects when taken at high doses (Zahoor, I., A. Shafi, and E. Haq, Pharmacological Treatment of Parkinson's Disease, in Parkinson's Disease: Pathogenesis and Clinical Aspects, T. B. Stoker and J. C. Greenland, Editors. 2018: Brisbane (AU)). Other treatments such as deep brain stimulation only ameliorate symptoms but do not stop the progression of disease (Dallapiazza, R. F., et al., Considerations for Patient and Target Selection in Deep Brain Stimulation Surgery for Parkinson's Disease, in Parkinson's Disease: Pathogenesis and Clinical Aspects, T. B. Stoker and J. C. Greenland, Editors. 2018: Brisbane (AU)). Therefore, better treatments for PD are urgently needed.

Current biologic PD treatments under investigation can be categorized into three types. 1) By modulating neuronal signaling to restore neurotransmitter imbalances. For example, gene therapies delivering AADC, an enzyme involved in dopamine synthesis, by viral vectors, e.g., AAV2-AADC (CN107106689A, relevant clinical trials@https://clinicaltrials.gov/ct2/show/NCT03065192?term=AADC&cond=PD&draw=2&rank=8) and a lentivirus-based gene therapy (ProSavin) have shown promising results in early stage clinical trials with mild improvements in the motor function of patients. 2) By expression of neurotrophic and regenerative factors to improve neuronal survival. For example, neurotrophic factor (GDNF) (clinical trial@https://clinicaltrials.gov/ct2/show/NCT04167540?term=GDNF&cond=PD&draw=2&rank=1) and Neurturin (NRTN) (clinical trial@https://clinicaltrials.gov/ct2/show/NCT00985517?term=Neurturin&draw=2&rank=1) are beneficial in the survival of neurons in the dopaminergic midbrain. 3) By targeting disease-associated genes or gene mutations, e.g., alpha synaptic nuclear protein (SNCA), glucocerebrosidase (GBA1) (clinical trial@https://clinicaltrials.gov/ct2/show/NCTO4127578?term=GBA1&cond=Parkinson+Disease&draw=2&rank=1), and the leucine-rich repeat kinase 2 (LRRK2). Several antisense oligonucleotides (ASOs)-, CRISPR-, and AAV-based gene therapies aiming to edit gene and/or regulate gene expression are currently in early-stage clinical trials.

Accordingly, there remains an unmet need for a treatment for PD, which can restore dopamine level in the striatum, clear α-syn, and preserve the dopaminergic transmission in the patient brain.

AAV-mediated gene replacement therapies may also have potential in treating other neurodegenerative disorders or neurodevelopmental diseases including, but not limited to multiple system atrophy (MSA), Lewy body dementia (LBD), Alzheimer's disease with Amygdalar restricted Lewy body (AD/ALB), Gaucher disease, AADC deficiency, and so on.

MSA is a rapidly progressive sporadic adult-onset neurodegenerative disorder. Clinical symptoms of MSA include parkinsonism (bradykinesia, rigidity, and postural instability which are similar to Parkinson's disease); cerebellar syndrome, and autonomic failure which is caused by brain stem nuclei degeneration. Two clinical subtypes are defined, MSA-P (Parkinsonism) and MSA-C(Cerebellar syndrome) based on the predominant symptomatology (Monzio Compagnoni, G. and A. Di Fonzo, Understanding the pathogenesis of multiple system atrophy: state of the art and future perspectives. Acta Neuropathol Commun, 2019. 7(1): p. 113). Main pathological features of MSA are the widespread neuronal and oligodendrocyte loss and gliosis in multiple brain regions.

The cause of MSA is still unclear, while the main pathogenic mechanism of MSA is the presence of argyrophilic filamentous glial cytoplasmic inclusions (GCIs), predominantly in oligodendrocytes. The GCIs are primarily composed of loosely packed filaments of α-synuclein protein that is phosphorylated at residue Ser129 and ubiquitinated. Based on its pathology and symptoms, currently available MSA treatments and therapies being developed are designed to target the loss of dopamine transmission, neuronal loss, and aggregation of α-synuclein, which are quite similar to approaches for treating PD.

Epidemiologic studies of MSA have shown a prevalence in the range of 3.4 to 4.9 per 100,000 people, increasing to 7.8 per 100,000 among people older than 40 years of age. Currently available treatments only alleviate symptoms. For example, dopamine derivatives (e.g., Medopa or Duopa) are used to reduce PD-like symptoms. However, these drugs are not as effective in treating MSA-P patients as they are in treating PD patients. In addition, some MSA-P patients only respond to these drugs at higher doses and they usually become less effective over time. Therefore, enhancement of dopamine production via delivery of AADC may not benefit MSA patients as much as shown for PD patients. Additional disease modifying therapies which could delay the disease progression are needed. Such therapies could potentially target three aspects of the MSA pathogenesis: (1) α-synuclein aggregation, (2) cellular dysfunction and loss, and (3) neuroinflammation. There are several active clinical-stage candidates, including an immunogenic peptide of α-synuclein (PD01 and PD03, NCT02270489), an α-synuclein aggregation inhibitor (Anle138b, NCT04208152; ATH434, NCT05109091), an antisense oligonucleotide (ASO) against α-synuclein (BIIB101, NCT04165486), and a neuroprotective factor like GDNF delivered by AAV (AAV2-GDNF, NCT04680065). All of the above-mentioned therapies that are under development target a single pathology or symptom. For a complex ND like MSA, simultaneously targeting multiple disease-causing pathologies could potentially provide better therapeutic effects or even disease modifying effects not only to alleviate its symptoms, but to slow the disease progression.

Gaucher disease (GD) is an autosomal recessive disorder mainly caused by the loss-of-function mutation of the GBA1 gene. GD has been classified into three types based on the absence (type 1), or presence and severity of the central nervous system (CNS) impairments (nGD, type 2 and type 3) (Bennett, L. L. and C. Fellner, Pharmacotherapy of Gaucher Disease: Current and Future Options. P T, 2018. 43(5): p. 274-309). Enzyme replacement therapy has been developed that is only effective for treating type 1 GD since the recombinant enzyme could not cross the blood-brain barrier (BBB). CNS-tropism AAV expressing GBA1 would be a promising approach to treat nGD. An active clinical trial (NCT04411654) sponsored by Prevail Therapeutics delivers codon-optimized GBA1 transgene via intra-cisterna magna (ICM) injection of rAAV9.

AADC deficiency (AADCD) is a rare, autosomal recessive disorder mainly caused by the loss-of-function mutation of the AADC gene. The abnormality of AADC protein leads to a severe deficiency of some of the most critical neurotransmitters for brain function, like serotonin, dopamine, norepinephrine and epinephrine. rAAV9 expressing functional wild-type AADC was developed to treat this disease by PTC therapeutics, called Upstaza™ (eladocagene exuparvovec) which has been approved by the EMEA (European Agency for the Evaluation of Medicinal Products) and UK for treating patients at an age of 18 months or older.

Taken together of the above evidence, NDs are complex, multifaceted and debilitating diseases that have huge impact on global public health. Development of innovated treatments that are disease modifying to slow the disease progression and alleviate patient sufferings is urgently needed.

### SUMMARY OF THE INVENTION

To develop better therapies for treating various NDs, the present inventors have modified the nucleotide sequences encoding for AADC, GBA1, and NTF such as CDNF and GDNF to optimize their expression when delivered into the human brain via rAAV vectors. In addition, the present inventors have developed rAAV vectors that express two genes of interest (GOI) in tandem to develop therapies targeting multiple disease pathologies synergistically to achieve greater therapeutic effects.

Therefore, in a first aspect, the present application provides a nucleotide sequence encoding one of the above listed GOIs, specifically AADC, GBA1, CDNF and GDNF, wherein the nucleotide sequence is a codon-optimized sequence as compared to the wild-type sequence of the GOI.

In a second aspect, the present application provides a nucleic acid construct comprising the nucleotide sequence of the first aspect operatively linked to a promoter.

In a third aspect, the present application provides a nucleic acid construct comprising two nucleotide sequences operatively linked to a promoter, wherein the first nucleotide sequence and the second nucleotide sequence encodes for two of (a), (b) and (c), respectively: (a) AADC, (b) GBA1, and (c) GDNF or CDNF. In a preferred embodiment, the nucleotide sequence encoding AADC is a codon-optimized sequence. In a specific embodiment, the nucleotide encoding for AADC is a nucleotide sequence of the first aspect. In another preferred embodiment, the nucleotide sequence encoding for GBA1 is a codon-optimized sequence. In a specific embodiment, the nucleotide encoding for GBA1 is a nucleotide sequence of the first aspect. In another preferred embodiment, the nucleotide sequence encoding for CDNF or GDNF is a partially codon-optimized sequence with the nucleotide region coding for the signal peptide unchanged. In a further embodiment, the nucleotide sequence encoding for CDNF or GDNF has a reduced number of CpG sites as compared to the wild-type coding sequence. In a further embodiment, the nucleotide sequence encoding for CDNF or GDNF does not contain any CpG island. In a specific embodiment, the nucleotide sequence encoding for CDNF or GDNF is a nucleotide sequence of the first aspect.

In a preferred embodiment, the nucleic acid construct of the third aspect comprises a linker sequence between the GOIs of first nucleotide sequence and the second nucleotide sequence. In a specific embodiment, the linker sequence is a 2A peptide. In a specific embodiment, the linker sequence is IRES.

In a fourth aspect, the present application relates to a rAAV vector comprising the nucleotide sequence of the first aspect, or the nucleic acid construct of the second or third aspect. In a preferred embodiment, the AAV vector is AAV9.

In a fifth aspect, the present application relates to a composition, e.g., a pharmaceutical composition, comprising the rAAV vector of the fourth aspect and a pharmaceutically acceptable excipient.

In a sixth aspect, the present application relates to a viral particle comprising the rAAV vector of the fourth aspect.

In a seventh aspect, the present application relates to a method of treating or preventing neurodegenerative disorders (NDs), e.g., Parkinson's disease (PD), Multiple system atrophy (MSA), Gaucher's disease (GD), AADC deficiency (AADCD), and other proteiopathies, in a subject in need thereof, comprising administering the rAAV vector of the fourth aspect to the subject.

In an eighth aspect, the present application relates to the use of the rAAV vector of the fourth aspect in treating or preventing neurodegenerative disorders, or in the manufacture of a medicament for treating or preventing neurodegenerative disorders, wherein the neurodegenerative disorders are particularly Parkinson's disease (PD), Multiple system atrophy (MSA), Gaucher's disease (GD), AADC deficiency (AADCD), and other proteinopathies.

The nucleic acid constructs of the present application comprise a combination of a promoter, a linker, and one or two therapeutic protein coding sequences specially optimized for rAAV vector so as to provide desirable levels of expression of the therapeutic proteins, e.g., one or two of AADC, GBA1, and CDNF/GDNF. Especially for the rAAV vector comprising two GOIs, such rAAV vector of the present invention can achieve co-expression of both therapeutic proteins at the desirable levels that in combination, they will work synergistically as novel therapies to treat the intended neurodegenerative diseases more effectively to achieve better therapeutic outcomes.

### BRIEF DESCRIPTION OF THE DRAWINGS

FIG. 1 shows the schematic of an exemplary construct containing the AADC wild-type coding sequence and GBA1 wild-type coding sequence linked by P2A peptide.

FIG. 2A and FIG. 2B show representative images of the Western blot (WB) results of AADC and GBA1 protein expression, alone or in combination in HEK293 cells. (FIG. 2A) Cells were transfected with constructs comprising coding sequence of AADC, GBA1, or AADC-P2A-GBA1 (AADC coding sequence linked to the GBA1 coding sequence with a P2A linker). (FIG. 2B) Cells were transfected with constructs comprising AADC and GBA1 coding sequences in different orders. GpA: GBA1-P2A-AADC; ApG: AADC-P2A-GBA1.

FIG. 3 shows representative images of the WB showing evaluation of the cleavage efficiency of different linkers used when expressing both AADC and GBA in the same construct.

FIG. 4 shows the schematic of constructs containing the wild-type AADC (AADC-WT) and the optimized GBA1 via a P2A linker, or the optimized AADC and the wild-type GBA1 (GBA1-WT) via a P2A linker under the control of a CBh hybrid promoter which consisted of a CMV enhancer, a chicken-3-actin promoter, and a hybrid intron.

FIG. 5 shows the AADC protein expression levels of different candidate constructs in the HEK293 cells, as determined by ELISA.

FIG. 6 shows the GBA1 protein expression levels of different candidate constructs in the HEK293 cells, as determined by ELISA.

FIG. 7 shows the AADC protein expression levels of different candidate constructs in the HEK293 cells, as determined by WB.

FIG. 8 shows the normalized AADC protein expression levels as shown in FIG. 7, presented as mean±S.E.M from three independent WB experiments.

FIG. 9 shows the GBA1 protein expression levels in the HEK293 cells expressed by different candidate constructs, as determined by WB.

FIG. 10 shows the normalized GBA1 protein expression levels as shown in FIG. 9, presented as mean±S.E.M from three independent WB experiments.

FIG. 11 shows the catalytic activity of AADC in HEK293 cells transfected with different candidate constructs.

FIG. 12 shows the catalytic activity of GBA1 protein expressed in the HEK293 cells transfected with different candidate constructs.

FIG. 13 shows the expression levels of both AADC and GBA1 proteins in the HEK293 cells transfected with different combination constructs.

FIG. 14 shows the relative activity of GBA1 protein in the HEK293 cells transfected with different combination constructs.

FIG. 15 shows the expression of AADC and GBA1 proteins in the HEK293 cells transduced with different candidate rAAV vectors.

FIG. 16 shows the representative WB images illustrating the AADC protein expression levels in the HEK293 cells transfected with different candidate constructs.

FIG. 17A and FIG. 17B show the schematic of constructs containing the wild-type AADC (AADC-WT) linked to an optimized CDNF (FIG. 17A) or GDNF (FIG. 17B) via a P2A linker under the control of a CBh promoter.

FIG. 18A and FIG. 18B show the CDNF protein expression levels in cell lysates and supernatants of the HEK293 cells transfected with different candidate constructs, as determined by WB. (FIG. 18A) WB images; (FIG. 18B) normalized data.

FIG. 19A and FIG. 19B show the CDNF protein expression levels in cell lysates and supernatants of the U87 cells transfected with different candidate constructs, as determined by WB. (FIG. 19A) WB images; (FIG. 19B) normalized data.

FIG. 20A and FIG. 20B show the CDNF protein expression levels in cell lysates and supernatants of the HEK293 cells (FIG. 20A) or U87 cells (FIG. 20B) transfected with different candidate constructs, as determined by ELISA.

FIG. 21 shows the protective effects of CDNF protein in the supernatants of the HEK293 cells transfected with different candidate constructs treated with either 1.5 mM MPP+ or 40 nM Rotenone, as determined by CCK-8 assay.

FIG. 22 shows the GDNF protein expression levels in cell lysates and supernatants of the HEK293 cells transfected with different candidate constructs, as determined by WB.

FIG. 23 shows the GDNF protein expression levels in cell lysates and supernatants of the HEK293 cells transfected with different candidate constructs, as determined by ELISA.

FIG. 24 shows the protective effects of GDNF protein in the supernatants of the HEK293 cells transfected with different candidate constructs treated with 1.5 mM MPP+, as determined by CCK-8 assay.

FIG. 25 shows the CDNF (left panel) or GDNF (right panel) protein expression levels in the supernatants of the HEK293 cells transfected with the indicated constructs.

FIG. 26 shows the AADC protein activity in the cell lysates of the HEK293 cells transfected with the indicated candidate constructs.

FIG. 27 shows the protective effects of CDNF in the supernatants of the HEK293 cells transfected with different candidate constructs treated with 1.5 mM MPP+ or 40 nM Rotenone, as determined by CCK-8 assay and LDH assay.

FIG. 28 shows the protective effects of GDNF protein in the supernatants of the HEK293 cells transfected with different candidate constructs treated with 1.5 mM MPP+ or 40 nM Rotenone, as determined by CCK-8 assay and LDH assay.

FIG. 29 shows both the AADC and NTF (CDNF or GDNF) protein expression levels in the cell lysates, and NTF levels in the supernatants of the U87-AAVR cells transduced with the indicated rAAV9 vectors.

FIG. 30 shows the relative activity of the AADC protein from the HEK293 and U87-AAVR cells transduced with the indicated rAAV9 vectors.

FIG. 31 shows the contralateral rotation numbers taken from study subjects of a mouse 6-OHDA induced PD model after dosing with EF1α-A11-GDNF MN and benchmark rAAV.

FIG. 32 shows the net rotation numbers taken from study subjects of a mouse 6-OHDA induced PD model after dosing with EF1α-A11-GDNF MN and benchmark rAAV.

FIG. 33A and FIG. 33B show the schematic of constructs containing the optimized GBA1 linked to an optimized CDNF (FIG. 33A) or GDNF (FIG. 33B) via a P2A linker under the control of a promoter (CAG or MBP promoter). “OP” refers to optimized nucleotide sequence.

FIG. 34 shows the moving velocity and distance measurements of the 1-methyl-4-phenyl-1,2,3,6-tetrahydropyridine/probenecid (MPTP/P) induced PD mice after dosing of CBh-A11G11 and benchmark A10-vy rAAV, respectively. Pre: data collected before MPTP modeling; Post MPTP/P: data collected after induction by MPTP/P and before AAV treatment; Post AAV: data collected post AAV treatment.

FIG. 35 shows the rearing behavior (standing by hindlimbs) taken from the study subjects of an α-synuclein A53T transgenic mouse model of PD after dosing of EtI7-A11G11 (EtI7) and CBh-A11G11 (CBh) rAAVs. Low: 4E+9 vg, High: 4E+10 vg.

FIG. 36 shows representative images of the WB showing the expression of AADC and GCase in the tissue samples collected from the study subjects of the α-synuclein A53T transgenic mouse model of PD after dosing of EtI7-A11G11 and CBh-A11G11 rAAVs. Low: 4E+9 vg, High: 4E+10 vg.

FIG. 37A and FIG. 37B show representative images of the WB (FIG. 37A) and the normalized data (FIG. 37B) showing the levels of phosphorylated and total α-synuclein in the tissue samples collected from the α-synuclein A53T transgenic mouse model of PD after dosing of EtI7-A11G11 and CBh-A11G11 rAAVs. Low: 4E+9 vg, High: 4E+10 vg.

FIG. 38A and FIG. 38B show the schematic of constructs comprising different promotors and the codon optimized GBA1.

FIG. 39 shows the survival curve (viability) of the CBE induced GD mice after the administration of G10-p, CAG-G11 and EF1α-G11 rAAVs, respectively.

FIG. 40A and FIG. 40B show the moving distance in open field test (FIG. 40A) and latency to fall in rotarod test (FIG. 40B) of CBE induced GD mice after the administration of G10-p, CAG-G11, and EF1α-G11 rAAVs, respectively, in different doses.

FIG. 41 shows the GCase activity in the striatum samples collected from the CBE induced GD mice after the administration of G10-p, CAG-G11, and EF1α-G11 rAAVs, respectively, in different doses.

FIG. 42 shows the representative WB images showing the protein expression of AADC and CDNF in cell lysates and CDNF protein levels in the supernatants of the HEK293 cells transfected with the indicated candidate constructs.

FIG. 43 shows the net rotation numbers (amphetamine induced ipsilateral rotation) of 6-OHDA induced PD model mice after dosing with the indicated doses of CAG-A11-GDNF MN, EF1α-A11-GDNF MN, and benchmark A10-vy rAAVs.

FIG. 44A and FIG. 44B show the AADC (FIG. 44A) and GDNF (FIG. 44B) protein expression levels in striatum samples collected from 6-OHDAinduced PD model mice after dosing with the indicated amounts of EF1α-A11-GDNF MN and benchmark A10-vy rAAVs, as determined by ELISA.

FIG. 45 shows the representative images showing the tyrosine hydroxylase (TH) levels in the nigra samples collected from the 6-OHDA induced PD model mice after dosing with the indicated amounts of EF1α-A11-GDNF MN and benchmark A10-vy rAAVs, as determined by immunohistochemistry (IHC). Blank: healthy control.

FIG. 46 shows the Arotation numbers (L-DOPA induced contralateral rotation) taken from the 6-OHDA induced PD model rats after dosing with the indicated doses of EF1α-A11-GDNF MN and benchmark A10-vy rAAVs. Blank represents healthy control.

FIG. 47 shows the representative WB images showing the protein expression of GCase and GDNF in cell lysates and GDNF protein levels in the supernatants of the U87-MG-AAVR cells treated with different candidate rAAV vectors.

FIG. 48 shows the representative WB images showing the protein levels of the high molecular weight α-syn (HMW) of the SH-SY5Y-AAVR-A53T cells treated with different candidate rAAV vectors.

FIG. 49 shows the representative WB images showing the protein expression of GCase and GDNF in cell lysates and GDNF protein levels in the supernatants of the HEK293 cells transfected with different candidate constructs.

### DETAILED DESCRIPTION OF THE INVENTION

Unless specifically defined elsewhere in this document, all of the technical and scientific terms used herein have the meaning commonly understood by one of ordinary skill in the art to which this invention belongs.

As used herein, including the appended claims, the singular forms of words such as “a”, “an”, and “the”, include their corresponding plural references unless the context clearly dictates otherwise.

In the context of the present disclosure, unless being otherwise indicated, the wording “comprise”, and variations thereof such as “comprises” and “comprising” will be understood to imply the inclusion of a stated element, e.g. an amino acid sequence, a nucleotide sequence, a property, a step or a group thereof, but not the exclusion of any other elements, e.g. amino acid sequences, nucleotide sequences, properties and steps. When used herein the term “comprise” or any variation thereof can be substituted with the term “contain”, “include” or sometimes “have” or equivalent variation thereof. In certain embodiments, the wording “comprise” also include the scenario of “consisting of”.

The term “gene” as used herein refers to a nucleic acid (such as DNA, e.g., genomic DNA or cDNA) and its corresponding nucleotide sequence encoding an RNA transcript. As used herein, terms with reference to genomic DNA can include intervening non-coding regions as well as regulatory regions, and may include both 5′ and 3′ terminus. In some instances, the term includes transcribed sequences, including 5′ and 3′ untranslated regions (5′-UTR and 3′-UTR), exons and introns. In some genes, the transcribed regions will contain an “open reading frame” encoding the polypeptide. In some instances, a “gene” comprises only the coding sequence (e.g., an “open reading frame” or “coding region”) necessary to encode a polypeptide. In some instances, the term “gene” includes not only transcribed sequences, but also non-transcribed regions, including upstream and downstream regulatory regions, enhancers, and promoters. A gene may refer to an “endogenous gene” or a native gene. A gene may refer to a “foreign gene” or a non-native gene. A non-native gene can refer to a gene not normally found in the host organism but introduced into the host organism by gene transfer. A non-native gene can also refer to a gene that is not in its natural location in the genome of an organism. A non-native gene can also refer to a naturally occurring nucleic acid that contains mutations, insertions and/or deletions (e.g., non-native sequences), e.g., a codon-optimized nucleotide sequence. In the context of the present application, by “GOI” it specifically refers to CDS region, namely the sequences coding for amino acids in a protein, unless being otherwise indicated.

The terms “polynucleotide”, “oligonucleotide” and “nucleic acid” are used interchangeably herein and refer to a polymeric form of nucleotides of any length. A polynucleotide can be exogenous or endogenous to a cell. A polynucleotide can exist in a cell-free environment. Apolynucleotide can be a gene or a fragment thereof. Apolynucleotide can be DNA. A polynucleotide can be RNA. A polynucleotide can have any three-dimensional structure and can perform any function, known or unknown. Apolynucleotide may contain one or more analogs (e.g., altered backbones, sugars or nucleobases).

“Cassette” or “expression cassette” as described herein refers to a DNA component included in a vector (e.g., a plasmid vector or viral vector), and consisted of one or more genes under the control of regulatory sequences to be expressed in a host cell transduced by the vector.

“Operatively linked” as described herein is used to describe that two or more components, particularly nucleotide sequences, are connected in a way that each of the components can perform their designated functions.

“AAV” refers to adeno-associated virus.

“AADC” refers to aromatic L-amino acid decarboxylase. AADC is also known as DOPA decarboxylase (DDC).

“GBA1” refers to glucocerebrosidase that cleaves the beta-glucosidic linkage of glucocerebroside. In some cases, “GBA1” can be interchangeably referred as “GBA”, “GCB” or “GLUC”.

“NTF” refers to neurotrophic factor, which is a group of supportive proteins facilitating the development and maintenance of neurons. Both CDNF and GDNF belong to neurotrophic factors.

“CpG island” refers to a region in the genome rich in CpG sites. “CpG site” refers to two consecutive nucleotides consisting of a cytosine (C) and a guanine (G) in a 5′ to 3′ direction.

“2A peptide” refers to a group of short (18-22 amino acids) self-cleaving peptides derived from viruses. 2A peptides create ribosome skipping during translation, leading to separation between the end of the 2A sequence and downstream protein.

“IRES” refers to internal ribosome entry site.

“Proteinopathy” refers to a neurodegenerative disorder with accumulation of structurally abnormal protein, for example, α-synuclein, leading to formation of aggregates or inclusions in axons of neurons or oligodendrocytes.

In the context of the present application, “subject” refers to an animal, preferably a mammal, such as a primate, such as a cynomolgus monkey, preferably a higher primate, such as a human. Unless otherwise stated, the term “subject” is interchangeable with the term “patient” or “individual” in the context of this application.

### AADC

The present disclosure provides rAAV vectors which deliver aromatic L-amino acid decarboxylase (AADC) gene alone, or in combination with another gene of interest, e.g., GBA1, CDNF or GDNF. The rAAV vectors delivering AADC can be used to treat NDs, such as PD or AADCD.

The biosynthetic pathway of dopamine requires both tyrosine hydroxylase (TH), which converts tyrosine to 1-3,4-hydroxyphenylalanine (L-Dopa), and aromatic L-amino acid decarboxylase (AADC), which decarboxylates L-Dopa to generate dopamine.

It is known that AADC levels decline as PD progresses, and that L-DOPA taken by patients cannot be efficiently converted to dopamine in the axonal terminals within striatum. This makes more frequent and increased doses of L-DOPA necessary in order to achieve sufficient clinical responses. However, increasing the dose of L-DOPA could bring unwanted side effects, such as L-Dopa-induced dyskinesias (LID).

The potential of intra-putaminal AADC delivery by AAV in treating PD has been reported and evaluated clinically (Bankiewicz, K. S., et al., Long-term clinical improvement in MPTP-lesioned primates after gene therapy with AAV-hAADC. Mol Ther, 2006. 14(4): p. 564-70; Christine, C. W., et al., Magnetic resonance imaging-guided phase 1 trial of putaminal AADC gene therapy for Parkinson's disease. Ann Neurol, 2019. 85(5): p. 704-714). Considering that restoring AADC levels will “rescue” and boost the L-DOPA therapeutic effect, and dopamine levels are the most critical factor for the recovery of motor function, AADC is included in the candidate constructs to restore the dopamine levels, which would be especially beneficial for AADC deficient patients.

AADC deficiency is caused by loss-of-function mutation of the AADC gene. The rAAV of the present application comprising AADC as the sole GOI or in combination with another GOI, so as to compensate the molecular defects leading to the disease.

In a preferred embodiment, the nucleotide sequence encoding AADC can be optimized for expression in a rAAV construct. The optimization can be codon optimization.

In one embodiment, the rAAV of the present application comprises a nucleotide sequence encoding AADC, which comprises or consists of the nucleotide sequence as shown by any one of SEQ ID Nos: 1-9, 11 and 46, preferably as shown by any one of SEQ ID Nos: 1-9 and 46 (optimized sequence), more preferably as shown by SEQ ID NO: 3, SEQ ID NO: 9 or SEQ ID NO: 46. In some embodiments, the rAAV comprising a nucleotide sequence encoding AADC as the sole GOI can be used to treat PD or AADCD.

In some embodiments, the rAAV of the present application comprises a combination construct comprising both AADC and GBA1, e.g., for use in the treatment of PD. In one embodiment, the combination construct comprising a coding sequence of AADC and a coding sequence of GBA1. In some embodiments, the coding sequence of GBA1 is arranged at 5′ upstream of the coding sequence of AADC. In more preferred embodiments, the coding sequence of AADC is arranged at 5′ upstream of the coding sequence of GBA1. Preferably, the two coding sequences are arranged in frame and under the control of the same promoter. Preferably, the nucleotide sequence encoding GBA1 comprises or consists of the nucleotide sequence as shown by any one of SEQ ID Nos: 12-20, 45 or 47. Preferably, the nucleotide sequence encoding AADC comprises or consists of the nucleotide sequence as shown by any one of SEQ ID Nos: 1-9 and 46.

In one specific embodiment, the combination construct of AADC and GBA1 comprises a coding sequence of AADC as shown in SEQ ID NO: 46, and a coding sequence of GBA1 as shown in SEQ ID NO: 45. In one specific embodiment, the combination construct of AADC and GBA1 comprises from 5′ to 3′: a promoter sequence which is a truncated variant of EF1α promoter, e.g. the one as shown in SEQ ID NO: 56 (EFIt7); a coding sequence of AADC as shown in SEQ ID NO: 46; a coding sequence of GBA1 as shown in SEQ ID NO: 45; and a polyA sequence such as a hGH poly A sequence. In one specific embodiment, the combination construct of AADC and GBA1 comprises from 5′ to 3′ a promoter sequence as shown in SEQ ID NO: 60 (CBh promoter); a coding sequence of AADC as shown in SEQ ID NO: 46; a coding sequence of GBA1 as shown in SEQ ID NO: 45; and a polyA sequence such as a hGH poly A sequence.

In some embodiments, the rAAV of the present application comprises a combination construct comprising both AADC and a NTF selected from GDNF or CDNF, e.g., for use in the treatment of AADCD. In one embodiment, the combination construct comprising a coding sequence of AADC and a coding sequence of either GDNF or CDNF. In some embodiments, the coding sequence of GDNF or CDNF is arranged at 5′ upstream of the coding sequence of AADC. In more preferred embodiments, the coding sequence of AADC is arranged at 5′ upstream of the coding sequence of GDNF or CDNF. Preferably, the two coding sequences are arranged in frame and under the control of the same promoter. Preferably, the nucleotide sequence encoding AADC comprises or consists of the nucleotide sequence as shown by any one of SEQ ID Nos: 1-9 and 46. Preferably, the nucleotide sequence encoding GDNF comprises or consists of the nucleotide sequence as shown by any one of SEQ ID Nos: 27-29. Preferably, the nucleotide sequence encoding CDNF comprises or consists of the nucleotide sequence as shown by any one of SEQ ID Nos: 23-25.

In some embodiments, the rAAV of the present application comprises a combination construct comprising a coding sequence of AADC and a coding sequence of CDNF, e.g., for use in the treatment of AADCD. In one specific embodiment, the combination construct of AADC and CDNF comprises a coding sequence of AADC as shown in SEQ ID NO: 3 or SEQ ID NO: 46, and a coding sequence of CDNF as shown in SEQ ID NO: 25 (CDNF-MN). In one specific embodiment, the combination construct of AADC and CDNF comprises from 5′ to 3′: a CAG promoter sequence as shown in SEQ ID NO: 58 (CAG); a coding sequence of AADC as shown in SEQ ID NO: 3; and a coding sequence of CDNF as shown in SEQ ID NO: 25 (CDNF-MN). In one specific embodiment, the combination construct of AADC and CDNF comprises from 5′ to 3′: an EF1α promoter sequence as shown in SEQ ID NO: 57 (EF1α); a coding sequence of AADC as shown in SEQ ID NO: 46; and a coding sequence of CDNF as shown in SEQ ID NO: 25 (CDNF-MN). In one specific embodiment, the combination construct of AADC and CDNF comprises from 5′ to 3′: an EF1α promoter sequence as shown in SEQ ID NO: 57 (EF1α); a coding sequence of CDNF as shown in SEQ ID NO: 25 (CDNF-MN); and a coding sequence of AADC as shown in SEQ ID NO: 46.

In some preferred embodiments, the rAAV of the present application comprises a combination construct comprising a coding sequence of AADC and a coding sequence of GDNF, e.g., for use in the treatment of AADCD. In one specific embodiment, the combination construct of AADC and GDNF comprises a coding sequence of AADC as shown in SEQ ID NO: 46, and a coding sequence of GDNF as shown in SEQ ID NO: 29 (GDNF-MN). In one specific embodiment, the combination construct of AADC and GDNF comprises from 5′ to 3′: a EF1α promoter sequence as shown in SEQ ID NO: 57 (EF1α); a coding sequence of AADC as shown in SEQ ID NO: 46; and a coding sequence of GDNF as shown in SEQ ID NO: 29 (GDNF-MN).

### GBA1

The present disclosure provides rAAV vectors which deliver glucocerebrosidase 1 (GBA1) gene alone, or in combination with another gene, e.g., ADCC, CDNF or GDNF. The rAAV vector delivering GBA1 can be used for treating NDs, including Gaucher disease (GD), Parkinson's disease (PD) and Multiple system atrophy (MSA). For example, the rAAV vector delivering GBA1 alone can be used to treat type 2 and type 3 Gaucher disease (GD) or PD. For example, the rAAV vector delivering both GBA1 and NTF, such as GDNF can be used to treat MSA.

In a preferred embodiment, the nucleotide sequence encoding GBA1 can be optimized for expression by a rAAV construct. The optimization can be codon optimization.

In one embodiment, the rAAV of the present application comprises a nucleotide sequence encoding GBA1, which comprises or consists of the nucleotide sequence as shown by any one of SEQ ID Nos: 12-20, 22, 45, and 47, preferably as shown by any one of SEQ ID Nos: 12-20, 45 or 47 (codon-optimized sequence), more preferably as shown by SEQ ID NO: 15, SEQ ID NO: 16, SEQ ID NO: 20, SEQ ID NO: 45 or SEQ ID NO:47.

In some embodiments, the rAAV of the present application comprises a nucleotide sequence encoding GBA1, e.g., for use in the treatment of GD or PD. Preferably, the nucleotide sequence encoding GBA1 comprises or consists of the nucleotide sequence as shown by any one of SEQ ID Nos: 12-20, 45 or 47. In one specific embodiment, the rAAV comprises a coding sequence of GBA1 as shown in SEQ ID NO: 45. In one specific embodiment, the rAAV comprising a construct that comprises from 5′ to 3′: a CAG promoter sequence as shown in SEQ ID NO: 58 (CAG); a coding sequence of GBA1 as shown in SEQ ID NO: 45; and a polyA sequence such as a hGH poly A sequence. In one specific embodiment, the rAAV comprising a construct that comprises from 5′ to 3′: a EF1α promoter sequence as shown in SEQ ID NO: 57 (EF1α); a coding sequence of GBA1 as shown in SEQ ID NO: 45; optionally WPRE; and a polyA sequence such as a hGH poly A sequence.

In some embodiments, the rAAV of the present application comprises a combination construct comprising both GBA1 and a NTF selected from GDNF or CDNF, e.g., for use in the treatment of MSA. In some embodiments, the coding sequence of GBA1 is arranged at 5′ upstream of the coding sequence of NTF. In some embodiments, the coding sequence of NTF is arranged at 5′ upstream of the coding sequence of GBA1. Preferably, the two coding sequences are arranged in frame and under the control of the same promoter.

In some embodiments, the rAAV of the present application comprises a combination construct comprising both GBA1 and GDNF, e.g., for use in the treatment of MSA. Preferably, the nucleotide sequence encoding GBA1 comprises or consists of the nucleotide sequence as shown by any one of SEQ ID Nos: 12-20, 45 or 47. Preferably, the nucleotide sequence encoding GDNF comprises or consists of the nucleotide sequence as shown by any one of SEQ ID Nos: 27-29. In specific embodiments, the combination construct of both GBA1 and GDNF comprises a coding sequence of GBA1 as shown in SEQ ID NO: 45 or SEQ ID NO: 47, and a coding sequence of GDNF as shown in any one of SEQ ID NOs: 27-29. In one specific embodiment, the combination construct of GBA1 and GDNF comprises from 5′ to 3′: a CAG promoter sequence as shown in SEQ ID NO: 58 (CAG) or a MBP promoter sequence as shown in SEQ ID NO: 59 (MBP); a coding sequence of GBA1 as shown in SEQ ID NO: 45 or SEQ ID NO: 47; a coding sequence of GDNF as shown in SEQ ID NO: 29 (GDNF-MN); an optionally WPRE; and a polyA sequence such as a SV40 polyA or a bGH poly A. In one specific embodiment, the combination construct of GBA1 and GDNF comprises from 5′ to 3′: a CAG promoter sequence as shown in SEQ ID NO: 58 (CAG) or a MBP promoter sequence as shown in SEQ ID NO: 59 (MBP); a coding sequence of GDNF as shown in SEQ ID NO: 29 (GDNF-MN); a coding sequence of GBA1 as shown in SEQ ID NO: 45 or SEQ ID NO: 47; an optionally WPRE; and a polyA sequence such as a SV40 polyA or a bGH poly A.

### Neurotrophic Factors: CDNF and GDNF

The present disclosure provides rAAV vectors which deliver a neurotrophic factor, specifically CDNF or GDNF, alone, or in combination with another gene, e.g., ADCC or GBA1. The rAAV vector delivering a neurotrophic factor can be used for treating NDs.

The neuronal protective role of neurotrophic factors has been well-established. Both CDNF and GDNF proteins have been studied in the framework of PD (Nasrolahi, A., et al., Neurotrophic factors hold promise for the future of Parkinson's disease treatment: is there a light at the end of the tunnel?Rev Neurosci, 2018. 29(5): p. 475-489).

In one embodiment, AADC is co-expressed with CDNF or GDNF, respectively, by a rAAV vector. This is the first example that AADC and a neurotrophic factor are co-expressed in the same AAV vector and show significant efficacy in a mouse PD model.

In another embodiment, GBA1 is co-expressed with GDNF or CDNF by a rAAV vector.

In a preferred embodiment, the nucleotide sequence encoding CDNF or GDNF can be optimized for expression by a rAAV construct. The optimization can be codon optimization. The codon optimization can be limited to the coding region of the mature protein of GDNF or CDNF, without changing the nucleotide sequence corresponding to the signal peptide of CDNF or GDNF, or the nucleotide sequence corresponding to the pre-peptide GDNF. In a preferred embodiment, the codon-optimized CDNF or GDNF coding sequence has reduced numbers of CpG islands as compared to the wild-type coding sequence.

In one embodiment, the rAAV of the present application comprises a nucleotide sequence encoding CDNF, which comprises or consists of the nucleotide sequence as shown by any one of SEQ ID Nos: 23-25.

In one embodiment, the rAAV of the present application comprises a nucleotide sequence encoding GDNF, which comprises or consists of the nucleotide sequence as shown by any one of SEQ ID Nos: 27-29.

### Expression Cassette

The term “expression cassette” herein refers to a DNA component included in a vector (e.g., rAAV vector) and consisted of one or more, specifically one or two GOIs selected from AADC, GBA1, and NTF (CDNF or GDNF) gene under the control of regulatory sequences to be expressed in a host cell transduced by the vector.

The term “combination construct” in the context of the present application refers to a construct comprising two GOIs, specifically two GOIs selected from AADC, GBA1, and a NTF (CDNF or GDNF) gene. In preferred embodiments, the two gene coding sequences in a combination construct is under the control of the same promoter.

In one embodiment, the expression cassette of the present application is characterized by expression of one GOI, preferably expression of a codon-optimized sequence of GOI, in particular those as recited in the present disclosure.

In one embodiment, the expression cassette of the present application is characterized by co-expression of two GOIs spaced by a linker sequence. In a further embodiment, one or two of the GOIs is expressed via a codon-optimized sequence, in particular those as recited in the present disclosure. For example, the two GOIs can be selected from the following combinations: AADC+GBA1, AADC+CDNF, AADC+GDNF, GBA1+GDNF, and GBA1+CDNF.

By optimizing cDNA sequence (codon) of the AADC gene, the GBA1 gene, the CDNF gene and the GDNF gene, their regulatory sequences, and optionally the linker, the expression cassette of the present disclosure inserted into an AAV vector can achieve higher and more consistent protein expression or co-expression in neuronal cells in vitro or in vivo. For example, the expression cassette of the present disclosure shows better performance in expression of GOI(s) in human cell lines with neuronal identity, such as U87-MG or SH-SY5Y. For example, the expression cassette of the present disclosure shows better performance in expression of GOI(s) in vivo in neuronal cells at Striatum, Substantial nigra, or other CNS regions.

In one embodiment, as an essential part of the expression cassette, the present disclosure first provides a group of wild-type or codon-optimized nucleotide sequences encoding for AADC protein, and a second protein which is selected from a group consisting of GBA1 protein, CDNF protein and GDNF protein, specifically, the human AADC protein having an amino acid sequence as shown in SEQ ID NO: 31, the human GBA1 protein having an amino acid sequence as shown in SEQ ID NO: 32, the human CDNF protein having an amino acid sequence as shown in SEQ ID NO: 33, the human GDNF protein having an amino acid sequence as shown in SEQ ID NO: 34.

In one embodiment, as an essential part of the expression cassette, the present disclosure first provides a group of wild-type or codon-optimized nucleotide sequences encoding for GBA1 protein, and a second protein which is selected from a group consisting of CDNF protein and GDNF protein, specifically, the human GBA1 protein having an amino acid sequence as shown in SEQ ID NO: 32, the human CDNF protein having an amino acid sequence as shown in SEQ ID NO: 33, the human GDNF protein having an amino acid sequence as shown in SEQ ID NO: 34.

By “isolated nucleic acid”, it means a DNA or RNA which is removed from all or a portion of a polynucleotide in which the isolated polynucleotide is found in nature, or is linked to a polynucleotide to which it is not linked in nature. An isolated nucleic acid molecule “comprising” a specific nucleotide sequence may include, in addition to the specified sequence, operably linked regulatory sequences that control expression of the coding region of the recited nucleic acid sequences. Due to the codon degeneracy, one skilled in the art understands that any specific amino acid sequence can be coded by several different nucleotide sequences.

“Codon-optimized coding sequence” herein refers to a nucleotide sequence coding for a protein, such as AADC protein, GBA1 protein, CDNF protein or GDNF protein modified from their wild-type coding sequence accommodating codon bias. Optimization may be achieved by reducing sequence complexity, adjusting GC content, adjusting codon usage and/or avoiding rare codons. The coding sequence which has been codon optimized usually shows an increased translational efficiency of the gene of interest (GOI), leading to a higher protein expression. Tools (e.g., JCat) with embedded algorithm to design codon optimized coding sequence are readily accessible to those skilled in the art. In a preferred embodiment, the codon of the AADC coding sequence of the present application has a Codon Adaptation Index (CAI) greater than 0.8. CAI is a measure of codon bias. One skilled in the art would understand that the actual efficiency of any sequence generated by running an algorithm still needs to be verified by experiments.

In the preferred embodiments, the codon-optimized coding sequence for human AADC protein comprises or consists of a nucleotide sequence selected from SEQ ID No: 1-10 and 46, the codon-optimized coding sequence for human GBA1 protein comprises or consists of a nucleotide sequence of SEQ ID No: 12-21, 45, and 47, the codon-optimized coding sequence for human CDNF protein comprises or consists of a nucleotide sequence of SEQ ID No: 23-25, the codon-optimized coding sequence for human GDNF protein comprises or consists of a nucleotide sequence of SEQ ID No: 27-29.

Further, an expression cassette can comprise one or more regulatory sequences in addition to coding sequence. Regulatory sequence can be selected from one or more of promoter, enhancer, polyadenylation sequence, and translation termination signal. A certain combination of regulatory sequences of the present disclosure can achieve unexpected effect in improving the expression efficiency of the coding sequence.

“Promoter” refers to a DNA sequence enables initiation of transcription of a downstream gene under the control of said promoter. Promoters include but not limited to constitutive promoters, cell type-specific promoters, tissue-specific promoters, development stage-specific promoters. Promoter can be a naturally occurring promoter of a gene, a modified version of a naturally occurring promoter or a synthetic promoter.

In the preferred embodiments, the promoter of the present disclosure can be a constitutive promoter. In the preferred embodiments, the promoter can be a CBh promoter, an EFIla promoter, a CAG promoter, an MBP promoter (myelin basic protein promoter) or a promoter derived therefrom.

“Enhancer” is a regulatory DNA sequence which can enhance the transcription of the GOI in rAAV together with the promoter. In a preferred embodiment, the expression cassette of the present application comprises of an enhancer. More preferably, the enhancer can be a CMV enhancer, e.g., in a CBh promoter.

In some embodiments, intron sequences functioning as enhancers can be included. For example, an intron sequence originated from the intron of GOI can be included in the expression cassette.

In some cases, a promoter together with an enhancer and/or an intron sequence are collectively referred to as “promoter” or “promoter element”. In a preferred embodiment, the promoter is the CBh promoter. In another preferred embodiment, the promoter is consisted of the EFS promoter and an intron sequence.

Preferably, the intron sequence has a total length of about or less than 200 bp, about or less than 250 bp, about or less than 300 bp, about or less than 350 bp, about or less than 400 bp.

For example, the intron sequence of the present disclosure is derived from the gene of interest. For example, the intron sequence is consisted of one or more fragments derived from one or more intronic regions of the gene of interest.

In a preferred embodiment, the promoter or promoter/intron element has a length of no more than 1000 bp, no more than 900 bp, no more than 850 bp, no more than 800 bp, no more than 700 bp, no more than 600 bp, no more than 500 bp, or no more than 400 bp, due to the limited packaging capacity of AAV.

In some cases, when the intron sequence is derived from an intronic region of the gene of interest, it can be inserted into the coding sequence (e.g., codon-optimized coding sequence) at a position corresponding to the position where it locates in the gene in nature, e.g., between two exons, instead of locating at a position 5′-upstream of the coding sequence and constituting a promoter/intron element.

The Kozak consensus sequence (Kozak sequence), named after the scientist who discovered it, is a nucleic acid sequence motif present in most eukaryotic mRNA transcripts that functions as the protein translation initiation site. Kozak sequence ensures the protein is accurately and efficiently translated.

In one specific embodiment, the expression cassette comprises an CMV enhancer, a chicken β-actin promoter, the first coding sequence of the first GOI, a linker, the second coding sequence of the second GOI, and SV40 polyA, wherein the first coding sequence and the second coding sequence is independently selected from those disclosed herein and encode any of the following combinations of two GOIs: AADC+GBA1, AADC+CDNF, AADC+GDNF, GBA1+GDNF, and GBA1+CDNF.

### Linker Sequence

In one aspect, the present application provides a linker sequence that produces high efficiency and fidelity when the linker sequence is used to connect two coding sequences of the present application to be co-expressed by one rAAV vector of the present application.

As an example of linker sequence, a sequence coding for 2A peptide (such as P2A, F2A, or E2A) or IRES (full-length version referred as “ECMV IRES” herein, and a shorter truncated version referred as “mini IRES” herein) can be used to connect the two coding sequences of the present application. The position of GOI(s) relative to linker sequence can be adjusted so as to achieve desirable performance in protein expression and function. In preferred embodiments, a P2A linker sequence is used in a rAAV between the two GOIs.

In specific embodiments, the linker sequence of the present application comprises or consists of a nucleotide sequence as shown in SEQ ID NOs: 35, 37, 39, 41, 43 or 44.

### Promoter

The rAAV vector of the present application may comprise a promoter conventionally used in rAAV vectors, including naturally occurring promoters, variants or hybrids thereof.

In some embodiments, the promoter can be a truncated variant of the wild-type promoter. For example, the promoter can be a truncated version of EF1α promoter as shown in Table 2 of Example 6, having a nucleotide sequence as shown in any one of SEQ ID NOs: 50-56.

In some embodiments, the promoter of the present application may also be used together with an enhancer, which can be native to said promoter or not. For example, a CBh promoter is a hybrid of CMV enhancer and chicken β-actin (CBA) promoter. In this case, reference to a promoter implies the inclusion of an enhancer, which can be understood by one skilled in the art. In one embodiment, the rAAV vector of the present application comprises a CBh promoter, e.g. a CBh promoter having a nucleotide sequence as shown in SEQ ID NO: 60.

In another embodiment, to drive the co-expression of AADC and CDNF, or the co-expression of AADC and GDNF, an EF1α promoter is used, e.g. an EF1α promoter having a nucleotide sequence as shown in SEQ ID NO: 57. In another embodiment, to drive the co-expression of AADC and GBA1, a truncated EF1α promoter is used, e.g. a truncated EF1α promoter having a nucleotide sequence as shown in any one of SEQ ID NOs: 50-56. In another aspect, to drive the co-expression of GBA1 and GDNF, a CAG promoter having a nucleotide sequence as shown in SEQ ID NO: 58 or a MBP promoter having a nucleotide sequence as shown in SEQ ID NO: 59 (or its truncation) is used.

### Poly A Signal

The rAAV vector of the present application also contains a polyadenylation signal (Poly A).

For example, the Poly A sequence can be used in the present application includes SV40 polyA, human growth hormone (hGH) polyA, bovine growth hormone (bGH) polyA.

In one embodiment, the expression cassette of the present disclosure comprises a hGH poly A. In one embodiment, the expression cassette of the present disclosure comprises a hGH poly A when the promoter is an EF1α promoter or a variant thereof.

In some embodiments, a woodchuck hepatitis virus post-transcriptional regulatory element (WPRE) is placed downstream of the GOI and proximal to the polyadenylation signal.

### AAV Vector Serotype

In one preferred embodiment, the rAAV of the present application is AAV9 vector.

Based on a previous report, intra-parenchymal injected AAV9 showed better dispersion in the target site than other serotypes including AAV1, AAV2, AAV5, and AAV8 (Watakabe, A., et al., Comparative analyses of adeno-associated viral vector serotypes 1, 2, 5, 8 and 9 in marmoset, mouse and macaque cerebral cortex. Neurosci Res, 2015. 93: p. 144-57). Since human striatum is a relatively large brain region, to achieve broader coverage of genes delivered by AAV, molecular engineering of the wild-type AAV capsid is necessary.

The inventors have identified novel AAV serotypes with significantly improved tissue tropism than the currently known AAVs for the putamen, the intended target region for AAV-based gene therapy in treating PD. Therefore, in another preferred embodiment, the rAAV of the present application uses the novel AAV capsids.

### Exemplary Constructs

In one embodiment, the present application provides an expression cassette which comprises from 5′ to 3′:
(a) 5′ ITR;
(b) a promoter;
(c) optionally an intron sequence;
(d) a coding sequence of a GOI;
(e) a polyA, and
(f) 3′ ITR,
wherein the GOI is selected from AADC, GBA1, CDNF or GDNF, and the coding sequence is a codon-optimized sequence.

In another embodiment, the present application provides an expression cassette which comprises from 5′ to 3′:
(g) 5′ ITR;
(h) a promoter;
(i) optionally an intron sequence;
(j) a coding sequence of the first GOI;
(k) a linker sequence;
(l) a coding sequence of the second GOI;
(m) a polyA, and
(n) 3′ ITR,
wherein the first coding sequence and the second coding sequence are independently selected from wild-type coding sequences or codon-optimized sequences, e.g., those as recited in the present disclosure, and encode any of the following combinations of two GOIs: AADC+GBA1, AADC+CDNF, AADC+GDNF, GBA1+GDNF, and GBA1+CDNF, in which the two GOIs can be arranged in any order.

In the constructs indicated in the two above paragraphs, each of the element can be independently selected from those described in the present disclosure. For example, the first nucleotide sequence can be an AADC coding sequence as shown in any one of SEQ ID NOs: 1-9, 11, and 46; a GBA1 coding sequence as shown in any one of SEQ ID NOs: 12-20, 22, 45, and 47; or a CDNF or GDNF coding sequence as shown in any one of SEQ ID NOs: 23-30. For example, the second nucleotide sequence can be an AADC coding sequence as shown in any one of SEQ ID NOs: 1-9, 11, and 46; an GBA1 coding sequence as shown in any one of SEQ ID NO: 12-20, 22, 45, and 47; or a CDNF or GDNF coding sequence as shown in any one of SEQ ID NOs: 23-30. Preferably, the first nucleotide sequence can be an AADC coding sequence as shown in SEQ ID NO: 3 or 46; or a GBA1 coding sequence as shown in any one of SEQ ID NOs: 16, 45, and 47. Additionally or alternatively in a preferred embodiment, the second nucleotide sequence can be a CDNF coding sequence as shown in SEQ ID NO:25 or a GDNF coding sequence as shown in SEQ ID NO: 29. In some embodiments, the first nucleotide sequence and the second nucleotide sequence are linked by a P2A linker sequence.

### Pharmaceutical Composition

The term “pharmaceutical composition” refers to a composition suitable for delivering to a subject. The pharmaceutical composition of the present disclosure comprises the isolated nucleic acid, the rAAV vector or the viral particle of the present disclosure and a pharmaceutically acceptable excipient. Conventional pharmaceutically acceptable excipients are known in the art and can be solid or liquid excipients. In one embodiment, the pharmaceutical composition can be a liquid for injection.

### Delivery Methods

The terms “administration”, “administering”, “treating” and “treatment” as used herein, when applied to a subject, e.g., an animal, including human, or to cell, tissue, organ, or biological fluid, means contact of an exogenous pharmaceutical, therapeutic, diagnostic agent, or composition with the subject, cell, tissue, organ, or biological fluid. Treatment of a cell encompasses contact of a reagent with the cell, as well as contact of a reagent with a fluid, where the fluid is in contact with the cell. The term “administration” and “treatment” also include in vitro and ex vivo treatments, e.g., of a cell, by a reagent, diagnostic, binding compound, or by another cell.

In preferred embodiments, the rAAV vector of the present application can be delivered via intravenous, intra cerebroventricular, intrathecal or intra-striatum administration. In a specific embodiment, the rAAV vector is delivered via intra-striatum route. In another specific embodiment, the rAAV vector is delivered via intra-cerebroventricular route. In the most preferred embodiment, the treatment or administration is carried out intra-cerebroventricularly (ICV), such as by ICV injection.

The rAAV vector can be administered via a single dose or multiple doses. In a specific embodiment, the rAAV vector is administered via a single injection.

The dosage of the rAAV vector injection can be varied based on the administration route. For example, intra-parenchymal/intra-striatum injection, usually needs the delivery of a specific volume of rAAV to cover striatum as much as possible to achieve sufficient therapeutic effects, given that the loss of dopaminergic transmission in the striatum results in the movement problems of the PD patients. The dosage can also be varied based on the body weight of the subject. Therefore, the dose range can be within a broad scope which covers 1.5×10¹⁰-1.5×10¹⁴ vg/kg.

### Therapeutic Uses

The term “treat”, “treating” or “treatment” includes to cure or at least to alleviate the symptoms of a neurodegenerative disorder, such as PD, MSA, GD, AADCD or other proteinopathies.

A subject having any of these neurodegenerative disorders can be diagnosed by a well-trained neurologist based on the genetic background, medical history, symptoms and signs, as well as the results of neurological and physical examinations, according to the Clinical Diagnostic Criteria (Postuma, R. B., et al., MDS clinical diagnostic criteria for Parkinson's disease. Mov Disord, 2015. 30(12): p. 1591-601; Palma, J. A., L. Norcliffe-Kaufmann, and H. Kaufmann, Diagnosis of multiple system atrophy. Auton Neurosci, 2018. 211: p. 15-25).

The viral vector expressing one aforementioned gene or co-expressing two aforementioned genes can be used to treat a subject who has aforementioned neurodegenerative disorder. The subject can be a clinically diagnosed early-stage PD patient, with one or more mutations in the AADC gene, GBA1 gene or other PD genetic risk genes like SNCA. The subject can also be a clinically diagnosed late-stage PD patient, without or with one or more mutations in AADC gene, GBA1 gene or other PD genetic risk genes as aforementioned. The subject can be one who has previously been treated or is being treated with dopamine derivatives, like Medopa, with or without L-DOPA induced dyskinesia. The subject can also be one resistant to currently available treatments.

The subject can be a clinically diagnosed GD patient classified as type 2 or type 3, or classified as type 1 with central nervous system symptoms. The subject can also be a clinically diagnosed MSA patient, who have shown parkinsonism (bradykinesia with rigidity tremor or postural instability), cerebellar syndrome (gait ataxia with cerebellar dysarthria limb ataxia or cerebellar oculomotor dysfunction) or at least one feature suggesting autonomic dysfunction, with or without GBA1 gene mutations or reduced activity of GBA1 protein.

The subject can also be a clinically diagnosed MSA patient resistant to currently available treatments, like dopamine derivatives.

### EXAMPLES

To facilitate the understanding and utilization of the present invention, the merits of the present invention will be described in more details with reference to examples and appended drawings. However, it should be understood that the following examples only intend to exemplify the present invention without any intention in limiting the scope of the present invention. The scope of the present invention should be defined by the claims.

### Example 1. Determination of the Optimal Linker to Connect the Coding Sequences of AADC and GBA1

In this example, the performance of different linker sequences, specifically E2A (SEQ ID NO: 35), F2A (SEQ ID NO: 37), T2A (SEQ ID NO: 39), P2A (SEQ ID NO: 41), mini IRES (SEQ ID NO: 43), and ECMV TRES (SEQ ID NO: 44) was tested in the construct comprising the wild-type coding sequences of both AADC and GBA1.

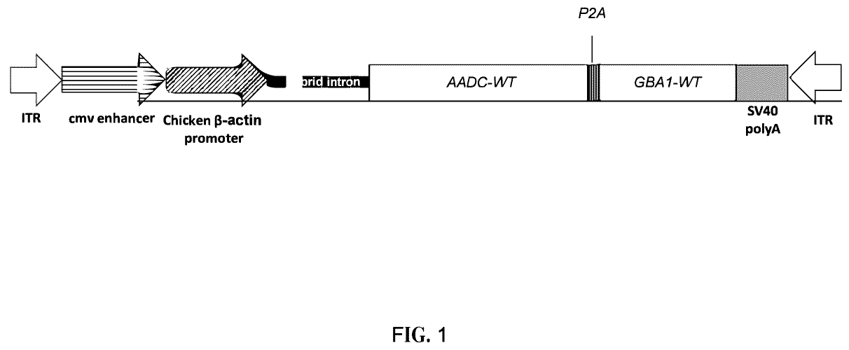

To construct an AAV vector containing both AADC and GBA1, the wild-type coding sequences of AADC (SEQ ID No:11) and GBA1 (SEQ ID No: 22) were connected by a self-cleaving P2A sequence in the order of AADC sequence followed by P2A then GBA1 sequence placed under the control of a CBh promoter (SEQ ID NO: 60) (FIG. 1). Also prepared is another construct with GBA1 coding sequence located upstream of the AADC coding sequence. Both constructs were introduced into a plasmid. The resulting plasmids are designated as CBh-AADC-P2A-GBA1-WPRE-SV40 pA (“AADC-P2A-GBA” or “ApG”) and CBh-GBA1-P2A-AADC-WPRE-SV40 pA (“GBA-P2A-AADC” or “GpA”), which were transfected into HEK293 cells (Procell, CL-0001). Plasmids containing the coding sequence of AADC or GBA1, alone (“AADC”, “GBA”) or together with the coding sequence of P2A (“AADC-P2A”, “P2A-GBA”), were also constructed and transfected into the same cells for comparison. Plasmid expressing GFP was used as a control.

Western blot was performed to detect the expression of AADC and GBA1 by each construct. Specifically, cells were collected in the lysis buffer (RIPA buffer, Thermo fisher 89901) 72 hours after transfection, denatured in 5×SDS-PAGE Sample Loading Buffer (Beyotime, P0015L) for 15 min at 95° C., separated in 10% SDS-PAGE gel (Sangon, #C631100), and blotted onto 0.22 μm PVDF membrane (Merck Millipore). The protein expression levels of AADC-WT, GBA1-WT, and the housekeeping gene GAPDH were detected by antibodies against the human AADC (Millipore, #AB1569), GBA1 (Sigma, #G4171), and GAPDH (CST, #2118), respectively. Long exposure and normal exposure were performed with the WBs.

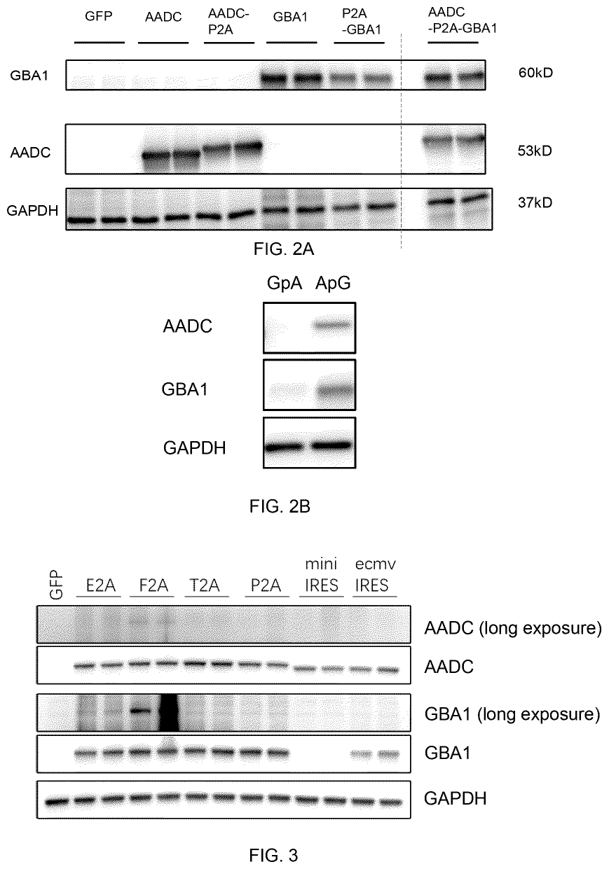

As shown in FIG. 2A, GBA1 protein expressed by the plasmid AADC-P2A-GBA1 (ApG) had almost the same molecular weight as GBA1 expressed by the plasmid comprising GBA1 alone, while the AADC derived from ApG was slightly larger than AADC expressed from the plasmid comprising AADC alone due to the P2A fragment attached thereto. This result indicates that self-cleaving 2A peptide is functional in this setting.

Unexpectedly, when the GBA1 gene was placed before (5′ upstream of) AADC gene, the expression of both genes was significantly decreased after transfection into the HEK293 cells (see FIG. 2B).

Additional linkers, including E2A, F2A, T2A, P2A, mini IRES, and ECMV IRES were evaluated in the same experiment setting as P2A. The results showed that except mini IRES, all the linkers evaluated produced both AADC and GBA1 proteins with the expected molecular sizes.

Additional tests were conducted by extending the exposure time to find out whether any fusion protein (AADC+GBA1) existed due to incomplete cleavage. Clear positive bands were detected with both AADC and GBA1 antibodies in the E2A and F2A constructs (see FIG. 3). The cleavage efficiency of the different linkers, ranking from high to low was ECMV RES, P2A, T2A, E2A, and then F2A. Using the full-length ECMV RES as a linker will result in a construct which would exceed the packaging capacity of a rAAV. Therefore, P2A was selected as the linker to use for our invention.

### Example 2. Codon Optimization to Improve the Protein Expressions of AADC and GBA1

In this example, nine sequences containing higher frequency codons were generated for AADC (referred as A1-A9, SEQ ID Nos: 1˜9) and GBA1 (referred as G1-G9, SEQ ID Nos: 12-20), respectively. All of these sequences have Codon Adaptation Index (CAI) greater than 0.85 as calculated by an online tool (https://www.genscript.com/tools/rare-codon-analysis). An AADC coding sequence and a GBA1 coding sequence disclosed in previous patent application publications were synthesized and used as references and named as A10 (SEQ IDNo: 10, disclosed in CN107106689A, Voyager Therapeutics Inc.) and G10 (SEQ ID No: 21, disclosed in WO2020210698A1, Prevail Therapeutics, Inc.), respectively. A10 encodes an M17V mutated AADC protein.

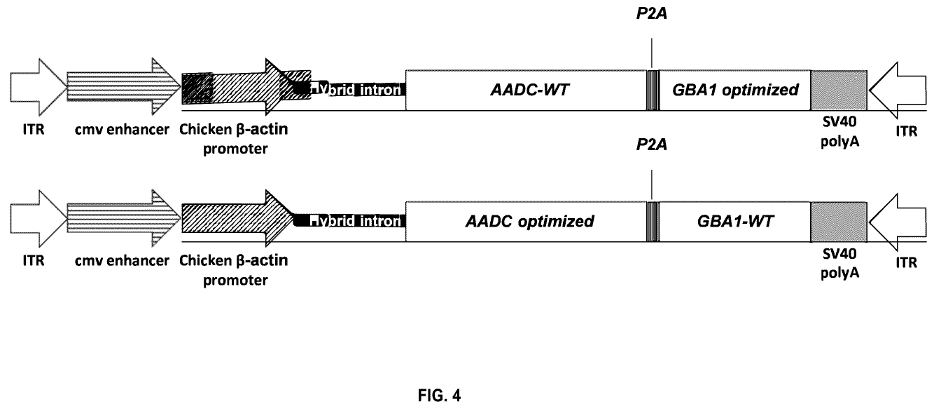

Twenty candidate constructs with configuration as shown in FIG. 4 were generated, by combining one of the codon-optimized AADC coding sequences (A1-A10) with the GBA1-WT (G0) sequence, or by combining the AADC-WT (A0) sequence with one of the codon-optimized GBA1 coding sequences (G1-G10) (see Table 1), with P2A used as the linker between the two coding sequences. Vectors containing G0 (wild-type GBA1 alone), A0 (wild-type AADC alone), and G0A0 were used as controls.

TABLE 1
Candidate recombinant constructs

| Group No. | Type of the combination | AADC | 2A | GBA |
|---|---|---|---|---|
| 1 | A0G0 | WT | P2A | WT |
| 2 | A1G0 | JCat | P2A | WT |
| 3 | A2G0 | BS | P2A | WT |
| 4 | A3G0 | GS | P2A | WT |
| 5 | A4G0 | GW1 | P2A | WT |
| 6 | A5G0 | NP | P2A | WT |
| 7 | A6G0 | SG | P2A | WT |
| 8 | A7G0 | SN | P2A | WT |
| 9 | A8G0 | TK | P2A | WT |
| 10 | A9G0 | TY | P2A | WT |
| 11 | A10G0 | Voyager | P2A | WT |
| 12 | A0 | WT | P2A | — |
| 13 | A0G1 | WT | P2A | JCat |
| 14 | A0G2 | WT | P2A | BS |
| 15 | A0G3 | WT | P2A | GS |
| 16 | A0G4 | WT | P2A | GW1 |
| 17 | A0G5 | WT | P2A | NP |
| 18 | A0G6 | WT | P2A | SG |
| 19 | A0G7 | WT | P2A | SN |
| 20 | A0G8 | WT | P2A | TK |
| 21 | A0G9 | WT | P2A | TY |
| 22 | A0G10 | WT | P2A | Prevail |
| 23 | G0 | — | P2A | WT |
| 24 | A0-P2A | WT | P2A | — |
| 25 | A10-vy | Voyager | — | — |
| 26 | G10-p | Prevail | — | — |

The vectors were transfected into HEK293 cells using Lipofectamine 3000 Transfection Reagent (Invitrogen, #L3000008) following the manufacturer's instructions. 48 hours after the transfection, cells were washed once with 1×PBS and harvested in the RIPA buffer (Thermo, 89901). The protein levels of AADC and GBA1 in the cell lysate were determined by a sandwich ELISA as described below

### ELISA to Detect AADC and GBA1 Protein Levels

1. On Day 1, 96-well plates were coated with the following capture antibodies in 100 μL coating buffer overnight at 4° C.: human DOPA Decarboxylase (DDC) monoclonal antibody (Sinobiological, 10560-R003, 2 μg/mL) for AADC samples, and GBA1 antibody (Abcam, ab55080, 1 μg/mL) for GBA1 samples.

2. The next day, the sample wells were washed with washing buffer 3 times. 300 μL blocking buffer (containing 2% BSA) was added and left for approximately 2 hrs at room temperature followed by 3 additional washing steps.

3. 100 μL assay buffer containing the following detection antibodies was added to each well: anti-DDC monoclonal antibody (tRP) (Sinobiological, 10560-R040, 1:1000 dilution) for AADC protein detection, and polyclonal anti-GBA antibody (Abcam, ab96246) for GBA1 protein detection. After incubation for approximately 1 hr at room temperature, the sample wells were washed for 3 times with the washing buffer.

4. To detect GBA1 protein expression, an RP-conjugated antibody (Goat Anti-Rabbit IgG-Fc Secondary Antibody (HRP) (SinoBiological, SSA003) was applied, followed by washing steps.

5. The sample wells were then incubated with 100 μL TMB solution (Solarbio, #PR1200) for approximately 20 min, then the reactions terminated by adding 100 μL stop solution (Solarbio, #C1058).

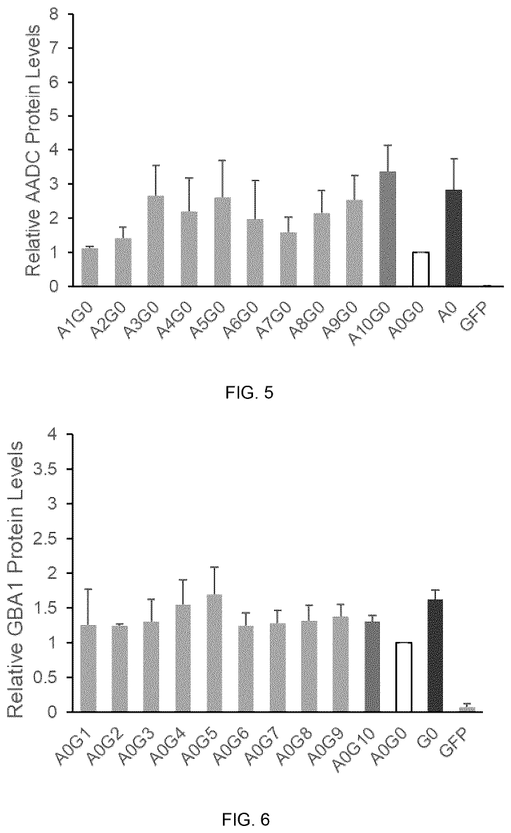

6. The absorbance (at an excitation wavelength of 450 nm and emission at 630 nm) of each sample was detected by a fluorimeter (SPECTRAmax Gemini XPS, Molecular Devices, San Jose, CA, USA) and the results are shown in FIG. 5 and FIG. 6.

As shown in FIG. 5, A3 and A9 were the top 2 candidates among the AADC coding sequences evaluated. A10 showed the highest AADC protein expression levels since it expressed an AADC mutant protein. As shown in FIG. 6, G4 and G5 were the top 2 candidates among the GBA1 coding sequences evaluated. In addition, both A0G4 and A0G5 expressed relatively higher GBA1 protein levels than the reference A0G10 construct containing a prior art reference GBA1 coding sequence G10.

### Western Blot

Western blot (WB) assays were also conducted to detect protein expression. Again, the candidate vectors (except G1, G2, G7, A1, A2, and A7) were transfected into HEK293 cells with Lipofectamine 3000 Transfection Reagent (Invitrogen, #L3000008).

48 hours after transfection, cells were harvested in RIPA buffer (Thermo, 89901), diluted and boiled (denatured) in 5×loading buffer (Beyotime, P0015L). All WB samples were separated on SDS polyacrylamide gels (BioRad, 1703932) and transferred onto a 0.22 m PVDF membrane (BIO-RAD, 1620177).

The membrane was incubated with anti-AADC (Millipore, #AB1569) or anti-GBA1(Sigma, #G4171) antibody, or antibody against the housekeeping gene GAPDH (CST, #2118) overnight at 4° C., followed by 2 hour incubation with HRP-linked secondary antibody at room temperature the next day.

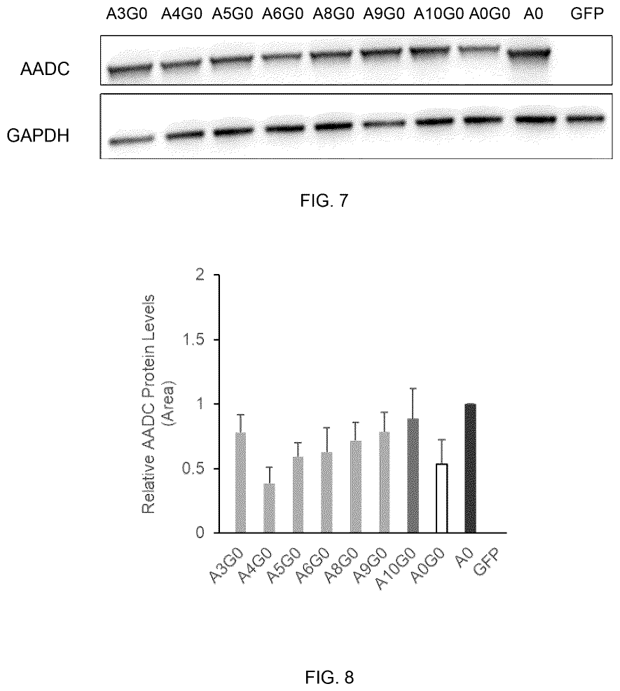

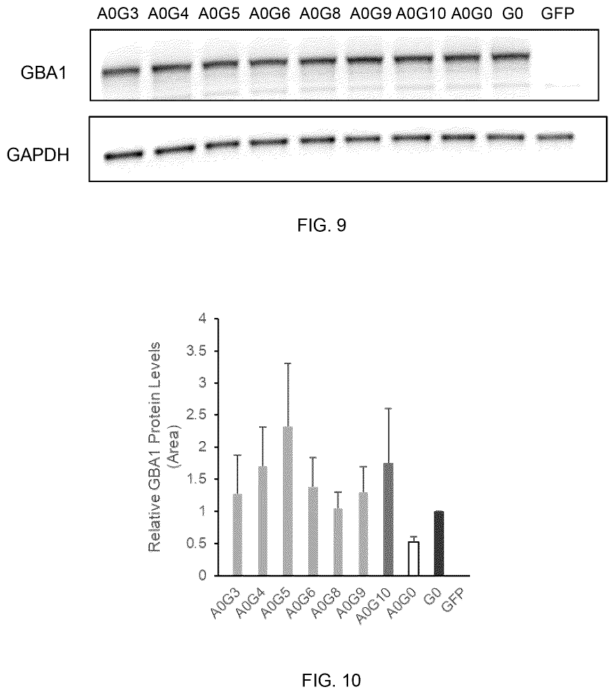

BeyoECL Moon kit (Beyotime, P0018F) was then applied to the membrane. Exposure and image capture were performed with a Tannon 5600 system, and the results are shown in FIGS. 7 and 9. The areas of the bands of AADC protein or GBA1 protein were determined by ImageJ 1.53 (NIH) and normalized to GAPDH levels. The GADPH normalized values were then normalized to that of A0 for the AADC group or G0 for the GBA1 group. The normalized data from three independent experiments were presented in FIGS. 8 and 10.

As shown by the WB results in FIGS. 7-10, it was found that A3G0 and A9G0 had the highest AADC protein expression among the codon optimized AADC candidates, and A0G4 and A0G5 were the best ones among the GBA1 candidates, consistent with results from the ELISA assays. A3, A9 (for AADC), and G4, G5 (for GBA1) were chosen as the codon-optimized sequences for further combination expression construct optimization.

### Example 3. Catalytic Activities of the Co-Expressed AADC and GBA1 Proteins

This example aims to determine whether the exogenously expressed AADC protein and GBA1 protein are catalytically active, indicating normal functions. Moreover, the catalytic activity could also be used as an additional evaluation criterion for the candidate sequences. The methods for examining AADC and GBA1 activities in the cell lysates are described as following.

### AADC Catalytic Activity (HPLC Analysis)

The AADC protein expressing cultures (A3G0, A9G0, A10G0, A0G0, A0) and the GFP protein expressing control sample were exposed to 100 M L-DOPA for 24 hrs. 100 μL of homogenization buffer (50 mM phosphate buffer, pH 7.4, containing 0.2 mM pyridoxyl phosphate and 0.2 mM pargyline) was added to each well, and cells were scraped from the bottom.

The cell homogenates were centrifuged for 10 min at 13,000 g, and 30 μL of each sample was used for the HPLC assay. Samples were subjected to HPLC/ECD to detect dopamine levels generated from catalytically active AADC protein expressed by different constructs. A standard curve of dopamine was used to calculate the relative AADC activities, as shown by the amount of dopamine transferred from L-DOPA in each sample.

### GBA1 Activity

The GBA1 protein-expressing cultures (A0G4, A0G5, A0G10, A0G0, G0) and the GFP protein-expressing control sample were tested for GBA1 protein activities. Recombinant human GBA (rhGBA) was used as a positive control.

1. rhGBA (Catalog #7410-GHB) was diluted to 0.2 ng/μL in Assay Buffer (50 mM Sodium Citrate, 25 mM Sodium Cholate, 5 mM DTT, pH 6.0).

2. The Substrate (4-Methylumbelliferyl-beta-D-glucopyranoside, Sigma, Catalog #M3633, 10 mM in DMSO) was diluted to 6 mM in the Assay Buffer.

3. 25 μL of cell lysate (total protein 100 ng/μL) or 0.2 ng/μL rhGBA was loaded into the wells of a 96-well plate. The reaction was started by adding 25 μL of 6 mM Substrate. 25 μL of the assay buffer was loaded to the standard curve well.

4. The plate was sealed and incubated at 37° C. for 3 hours.

5. After incubation, reaction was stopped by adding 50 μL of Stop Solution (0.5 M Glycine, 0.3 M NaOH (˜pH 10)) to each well.

6. Readings were recorded at excitation and emission wavelengths of 365 nm and 445 nm (top read), respectively, in an endpoint mode.

7. The specific activity was calculated as: (Adjusted Fluorescence*25)/(180 min*0.0025 mg. The results are shown in FIGS. 9 and 10.

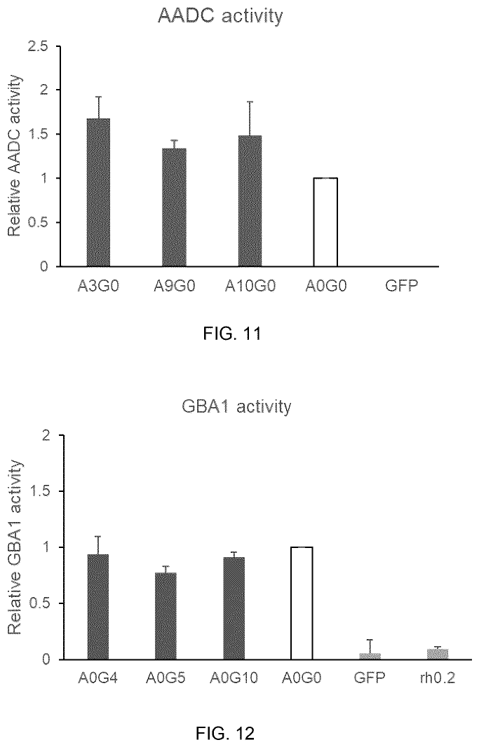

As shown in FIG. 11, the AADC protein expressed by construct A3G0 exhibited the highest catalytic activity among all the constructs evaluated. As shown in FIG. 12, the GBA1 protein expressed by construct A0G4 exhibited the highest catalytic activity.

### Example 4. Identification of Optimal Combination of Codon Optimized AADC and GBA1 Sequences

To identify the best combinations of codon optimized AADC and GBA1 sequences, the coding sequences of AADC and GBA1 with better performance as shown above were paired together and used to construct recombinant AAV vectors. Specifically, constructs A3G4, A9G4, A3G5 and A9G5 were generated. In addition, construct A11G11 was generated, which was based on codons of A3 and G4 with further optimization to completely remove the CpG islands from the AADC and GBA1 coding sequences. Two benchmark vectors were also constructed, A10-vy (SEQ ID NO: 48, codon A10 containing the promoter and other regulatory elements described in the same patent) and G10-p (SEQ ID NO: 49, codon G10 containing the promoter and other regulatory elements described in the same patent).

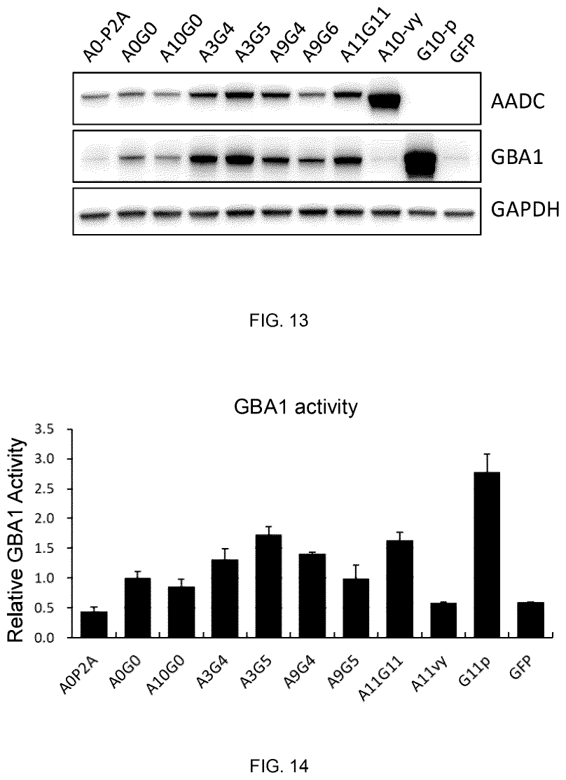

The above described vectors together with A0-P2A, A0G0, A10G0, A10-vy, and G10-p were transfected into HEK293 cells. The AADC and GBA1 protein expression and the catalytic activity of GBA were determined. It was found that among the combination constructs evaluated, A11G11, A3G5, and A9G4 expressed the highest levels of AADC protein as determined by WB analysis (FIG. 13). For GBA1 protein expression, constructs A3G5 and A11G11 expressed the highest levels of proteins that were also the most active, followed by construct A9G4 (FIG. 14).

### Example 5. Evaluation of AADC and GBA1 Protein Expression of the AAV9 Packaged Combination Constructs

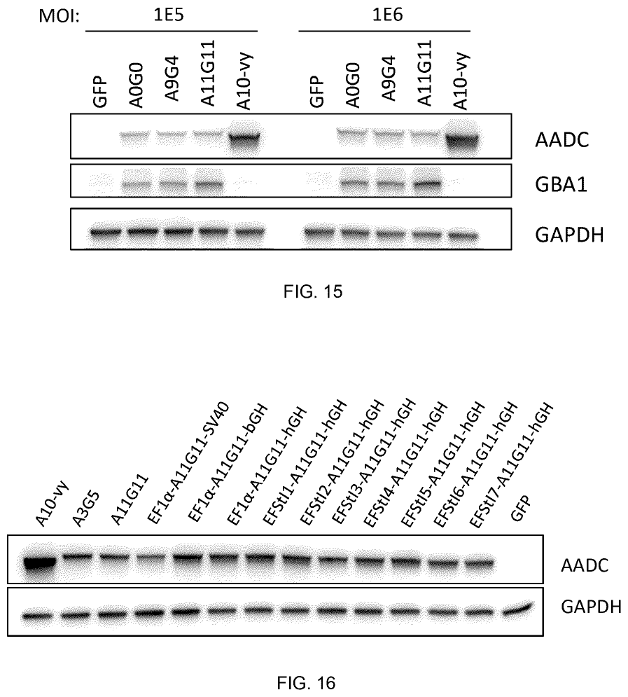

Constructs A0G0, A9G4, A11G11, and A10-vy were packaged into AAV in which serotype AAV9 was used. AAV9-A0G0, A9G4, and A11G11 together with a control AAV which only expressed GFP were administrated @MOI 1e5 and 1e6 into the U-87 MG cells which stably express AAV receptor to increase the transduction efficiency. Five days post infection, the AADC and GBA1 protein expression levels in these cell samples were determined by WB analysis. As shown in FIG. 15, cells transduced with the A11G11 AAV virus expressed higher levels of AADC and GBA1 proteins than the A9G4 packaged AAV virus. It was observed that the AADC protein expression was significantly higher in the A10-vy AAV virus transduced cells, most likely due to the stronger promoter used in the A10-vy construct.

### Example 6. Optimization of the Regulatory Elements Used in the Combination Expression Constructs

To further improve the protein expression of the combination constructs, the promoter and poly A sequences used were also optimized. The CBh promoter in A11G11 was replaced with the EF1α promoter (SEQ ID NO: 57), and the SV40 polyA tail was replaced with either bovine growth hormone poly A (bGH) or human growth hormone poly (hGH) shown in Table 2. Since EF1α-A11G11-hGH exceeded the packaging capacity of AAV, its length was reduced by truncating part of the EF1α promoter, resulting in seven different truncations (Nos. 5-11) as shown in Table 2.

The constructs in Table 2 together with A3G5, A10-vy, and GFP were transfected into the HEK293 cells. The AADC protein expression levels were determined by WB analysis. Among the first group of constructs evaluated, EF1α-A11G11-hGH expressed the highest levels of AADC protein. Among the constructs with truncated EF1α promoter (EFStI1-7, SEQ NO: 50-56), construct EFStI2 expressed the highest levels of AADC protein (FIG. 16).

TABLE 2
The combination constructs with EF1a and truncated EF1a promoter.

| No. | Vector | Promoter | PolyA | Length |
|---|---|---|---|---|
| 1 | CBh-A11G11-SV40 | CBh | SV40 | 4381 |
| 2 | EF1a-A11G11-SV40 | EF1a | SV40 | 4735 |
| 3 | EF1a-A11G11-bGH | EF1a | Bovine GH | 4812 |
| 4 | EF1a-A11G11-hGH | EF1a | Human GH | 5081 |
| 5 | EFStI1-A11G11-hGH | Truncated version 1 | Human GH | 4942 |
| 6 | EFStI2-A11G11-hGH | Truncated version 2 | Human GH | 4842 |
| 7 | EFStI3-A11G11-hGH | Truncated version 3 | Human GH | 4742 |
| 8 | EFStI4-A11G11-hGH | Truncated version 4 | Human GH | 4642 |
| 9 | EFStI5-A11G11-hGH | Truncated version 5 | Human GH | 4542 |
| 10 | EFStI6-A11G11-hGH | Truncated version 6 | Human GH | 4442 |
| 11 | EFStI7-A11G11-hGH | Truncated version 7 | Human GH | 4342 |

### Example 7. In Vivo Efficacy of AAV Candidates Expressing Optimized AADC and GBA1 Sequences in PD Mouse Models

The therapeutic effects of the AAVs expressing optimized AADC and GBA1 were evaluated in PD mouse models so as to determine if the candidate AAV can rescue the motor deficiency of PD animals.

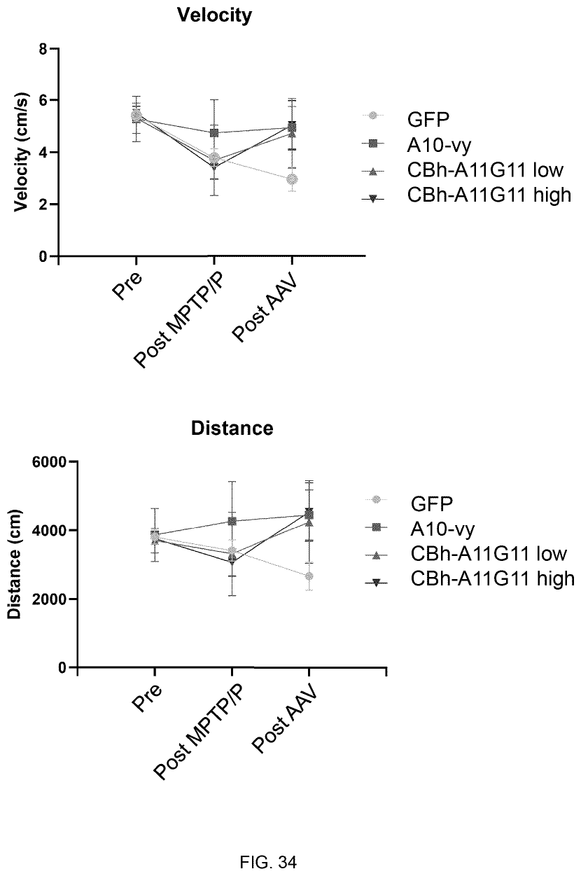

First, a widely used chemical-induced PD mouse model was used. The mice were treated by MPTP/probenecid. Candidate rAAV vector CBh-A11G11 was administrated at a higher dose of 2E+10 vg/per animal and a lower dose of 2E+09 vg/per animal into the striatum of the PD mouse model (“CBh-A11G11 low” and “CBh-A11G11 high”, respectively) once. rAAV vector A10-vy (2E+09 vg/per animal) was injected in a similar way as benchmark control (“A10-vy”), and rAAV vector expressing GFP was used as negative control (“GFP”). The motor function of the mice was evaluated by measuring the moving velocity and travelling distance before PD modeling (before induction with MPTP/P; “Pre”), as well as before and after administration of respective rAAV vectors (“Post MPTP/P” and “Post AAV”, respectively). The “Pre” behavioral tests took 6 days followed by the daily MPTP/probenecid administration for 35 days. After 3-day environmental habituation, the subjects underwent the same behavioral tests (“Post MPTP/P”) followed by rAAV injection. 21 days post rAAV injection, “Post AAV” behavioral tests were performed. The results are shown in FIG. 34.

As shown in FIG. 34, the moving velocity of the mice decreased after using MPTP/probenecid. The mice to be treated with A10-vy (N=3) showed less severe loss in speed as compared to other mice. All mice, expect for those to be treated with A10-vy, showed shortened moving distance. After treatment with corresponding rAAV, the motor function of the mouse as negative control (N=5) showed persistent decline, representing by continuously reducing moving velocity and distance. On the contrary, mice treated with both low (N=5) and high dose (N=7) of CBh-A11G11-SV40 showed restoration of moving velocity and distance, with the higher dose showing a slightly better result than the lower dose. These results suggested that CBh-A11G11-SV40 achieved significant effect in rescuing the deficiency in moving velocity and travelling distance in a PD mouse model induced by MPTP/probenecid. Given the damage to the moving capacity of the mouse treated with A10-vy was less severe as compared to other mice, it seemed that CBh-A11G11-SV40 achieved a better effect in rescuing the deficiency in moving velocity and travelling distance in a PD mouse model induced by MPTP/probenecid as compared to the benchmark.

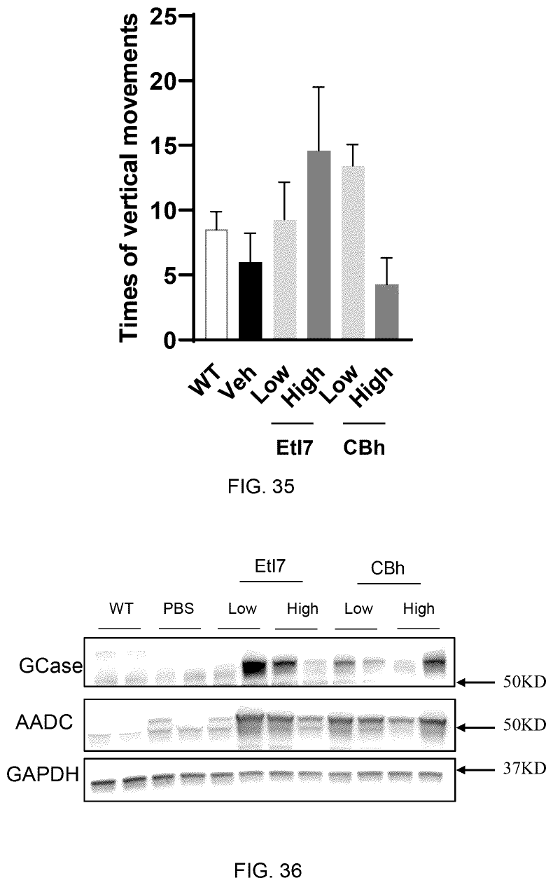

Then a transgenic PD mouse model was used to verify the results. The mice were genetically engineered to overexpress mutated α-synuclein (point mutation A53T) to recapitulate the PD pathology. Two rAAVs, EFSIt7-A11G11-hGH (EtI7) and CBh-A11G11-SV40 (CBh), were generated based on the construct A11G11 by using different promoters and polyA tails. The rAAVs were administered to the mice by intra-striatal injection, at a low dose of 4E+9 vg and a high dose of 4E+10 vg. Wild-type mouse without any treatment and A53T mouse treated with vehicle (PBS) were included as controls. The motor function was determined by measuring the times of vertical movements (rearing by the hindlimbs) and the results are shown in FIG. 35.

As shown in FIG. 35, the mice treated with vehicle (N=6) showed less times of vertical movements as compared to the WT mouse (N=8), indicating damage in motor function. Mice treated with both high (N=7) and low doses (N=7) of EFSIt7-A11G11-hGH (EtI7) showed better performance than Veh, with the high dose showing a significantly increased result. Mice treated with low dose of CBh-A11G11-SV40 (CBh) (N=7) also showed increased times of vertical movements as compared to both WT and vehicle A11G11-based rAAVs. The increased times of vertical movements indicated the recovery of motor function.

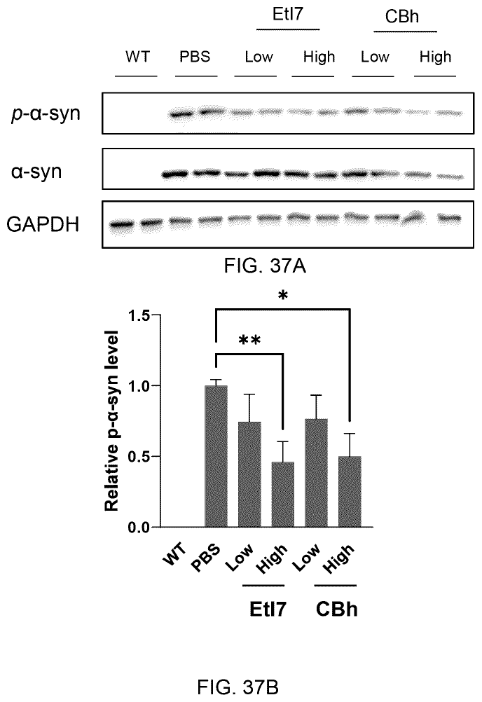

The brain tissue samples of A53T mice were collected for determining the protein expression. The expression of AADC and GCase proteins in the striatum was determined by WB. As shown in FIG. 36, all the treated groups showed expression of AADC and GCase. Additionally, the level of phosphorylated α-synuclein and total α-synuclein in the striatum were measured. The level of phosphorylated α-synuclein was thought to be the biomarker for PD pathology. As shown in FIGS. 37A and 37B, both candidate rAAV vectors decreased the phosphorylated α-synuclein level in a dose-dependent manner, indicating amelioration of PD pathology. The exemplary bands in FIG. 37A were from 2 representative subjects in each group. The relative phosphorylated α-synuclein level as shown in FIG. 37B was the mean value of all the subjects in the indicated group.

### Example 8. In Vivo Therapeutic Efficacy of AAV Candidates Expressing Optimized GBA1 Sequence in GD Mouse Model

The therapeutic effects of the AAVs expressing optimized GBA1 were tested in GD mouse models.

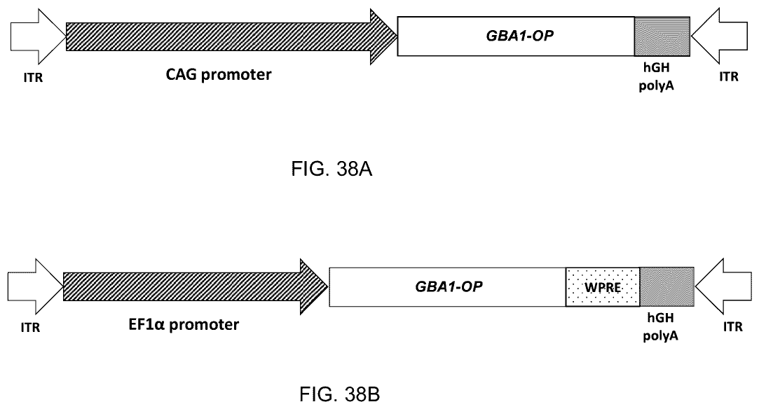

Two candidate vectors which expressed optimized GBA1 (G11) were constructed as shown in FIG. 38. The first one was designed to comprise the CAG promoter, G11 codon, and hGH polyA (“CAG”). The second one was designed to comprise the EF1α promoter, G11 codon, WPRE, and hGH polyA (“EF1α”). Both vectors were packaged into AAV9 as rAAV The candidate rAAV vectors were injected into the lateral ventricle of a chemical-induced GD mouse model and a genetically engineered GD mouse model to evaluate their effects.

The chemical-induced mouse model was CBE-induced GD mouse model. The GCase activity was inhibited by conduritol B epoxide (CBE), a specific irreversible GCase inhibitor to mimic the loss-of-function mutation in GBA1 gene. At postnatal Day 2 (P2), the mice were treated with either PBS, AAV9-GFP (negative control) or three different doses of candidate rAAV vectors (Low, 2.75E+09 vg; Medium, 8.8E+09 vg; High, 2.8E+10 vg, per animal) via ICV injection. G10-p was used as the reference. To leave sufficient time for the gene to express, disease modeling was initiated at P8. 37.5 mg/kg of CBE was injected into the mice via IP injection once a day. The in-life assessment was performed until P28.

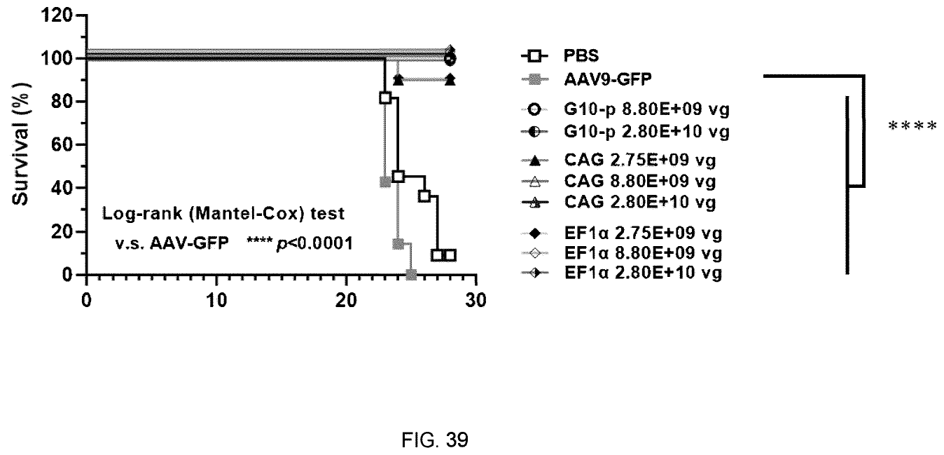

FIG. 39 shows the viability of GD mice until P28. As seen, the percentage survival of PBS group and negative control group dropped rapidly after P22. The negative control group has no animal survive at P25. On the contrary, both candidate vectors as well as the reference G10-p significantly improved the viability of GD mice.

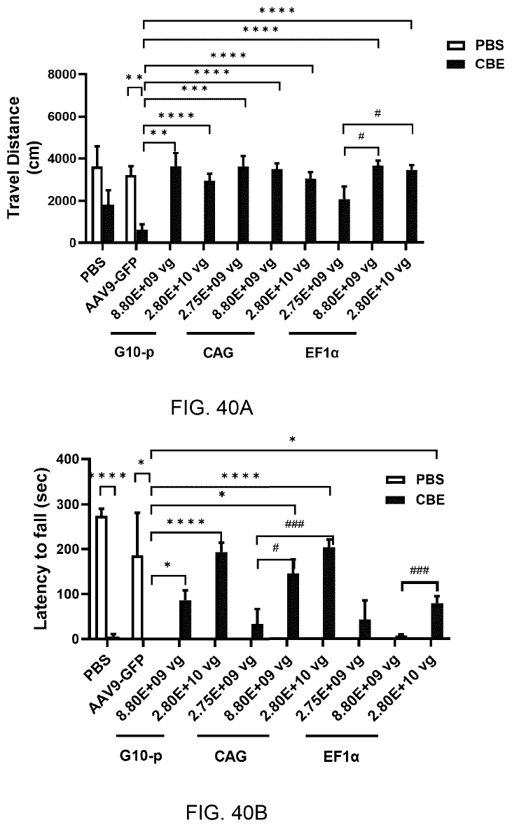

FIG. 40 shows the change in motor function of the study animals. Both candidate vectors rescued the locomotor deficiency and motor incoordination induced by chronic CBE administration. As shown in FIGS. 40A and 40B, CAG increased the moving distance in open field test at all doses and enhanced the motor coordination in Rotarod at medium and high doses, while EF1α increased the moving distance at medium and high doses, but improve the motor coordination only at high dose.

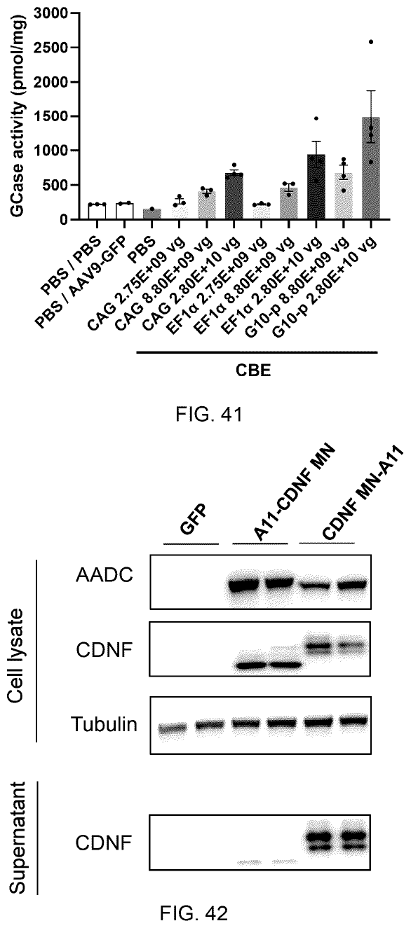

At postnatal day 30, the brains samples were collected for determining the protein activity. The brain samples were homogenized in the lysis buffer (N-PER Neuronal Protein Extraction Reagent, Thermo Scientific #87792) containing Protease inhibitor (Roche #11697498001) to generate lysate samples. The GCase activity in these lysate samples was determined by the GBA1 activity assay as mentioned above. It was found that the GCase activity was increased dose-dependently in all of the candidate vector-treated groups (FIG. 41).

### Example 9. Codon-Optimized Human CDNF Sequences for AADC Combination Constructs

In this Example, AAV vectors were designed to co-express AADC and a gene encoding NTF, specifically CDNF. P2A was used as the linker sequence between the two coding sequences.

To determine the optimal coding sequence of CDNF, the wild-type AADC coding sequence (AADC WT) was linked via P2A with different codon-optimized CDNF sequences. Codon optimization was conducted based on different algorithm.

Two codon-optimized CDNF sequences with high CAI (>0.85) were obtained and named as CDNF GS and CDNF SA, respectively. Both sequences were subjected to codon optimization except for the sequence fragment coding for the signal peptide (Nucleotide positions 1-72 of the nucleotide sequence as shown in SEQ ID NO: 26). In CDNF SA, the 9 nucleotides encoding for three amino acids immediately after the signal peptide (nucleotide positions 73-81) were left unchanged.

Meanwhile, another candidate CDNF sequence named as CDNF manual (CDNF MN) was obtained based on the sequence of CDNF SA by manually identifying and reducing the “CG” components in the mature protein coding sequence (nucleotide positions 82-561). As a result, all of the CpG islands were intentionally removed from CDNF MN sequence.

For this study, an AAV backbone vector, containing from 5′ to 3′, 5′ ITR, a CBh promoter, AADC WT coding sequence, a P2A sequence, a SV40 poly A, and a 3′ ITR was prepared.

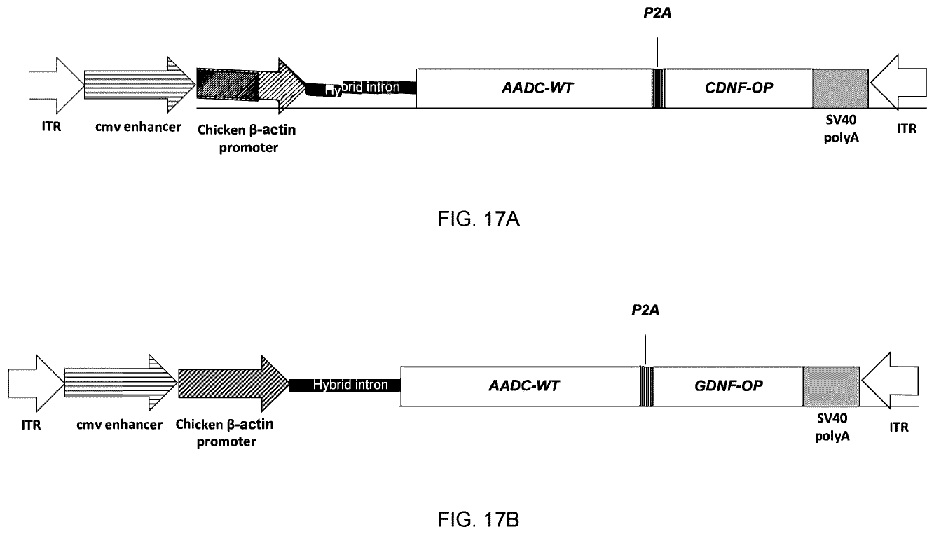

Sequences of CDNF GS, CDNF SA, CDNF MN and CDNF wild type (CDNF WT) were inserted into the aforementioned backbone vector respectively 3′ of the P2A linker sequence (FIGS. 17A and B). The vectors obtained were named ApC-GS, ApC-SA, ApC-MN, and ApC-WT. A reference vector comprising CDNF WT Alone (no AADC) was also constructed.

The 5 plasmids described above and one negative control plasmid only expressing GFP were transfected into HEK293 cells using Lipofectamine 3000 Transfection Reagent (Invitrogen, #L3000008). 72 hours after transfection, the supernatant was centrifuged at 2,000 rpm for 10 minutes to remove debris in the medium, 40 μL of supernatant and 10 μL 5× loading buffer (Beyotime, P0015L) were then transferred into a new 1.5 mL tube and incubated for 5 min at 95° C. Cells were washed once with 1×PBS and harvested in the RIPA buffer (Thermo, 89901).

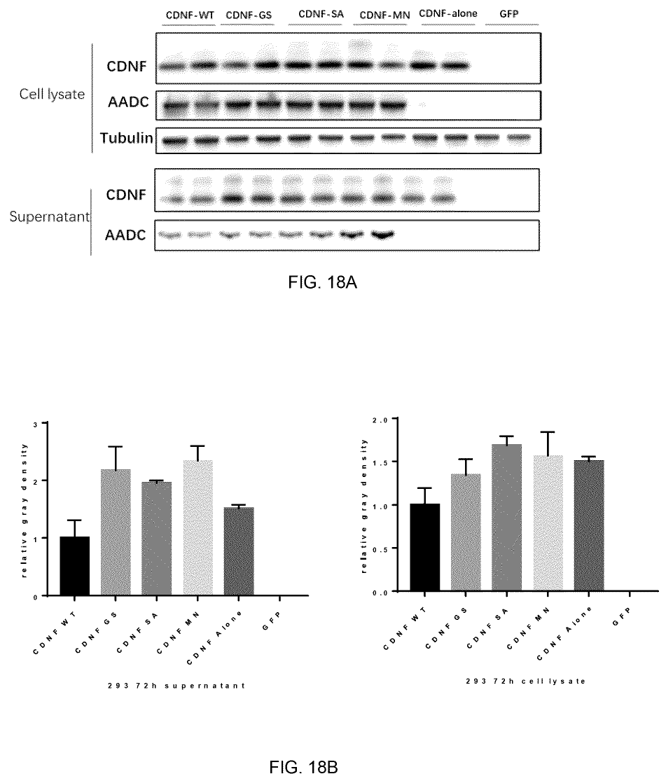

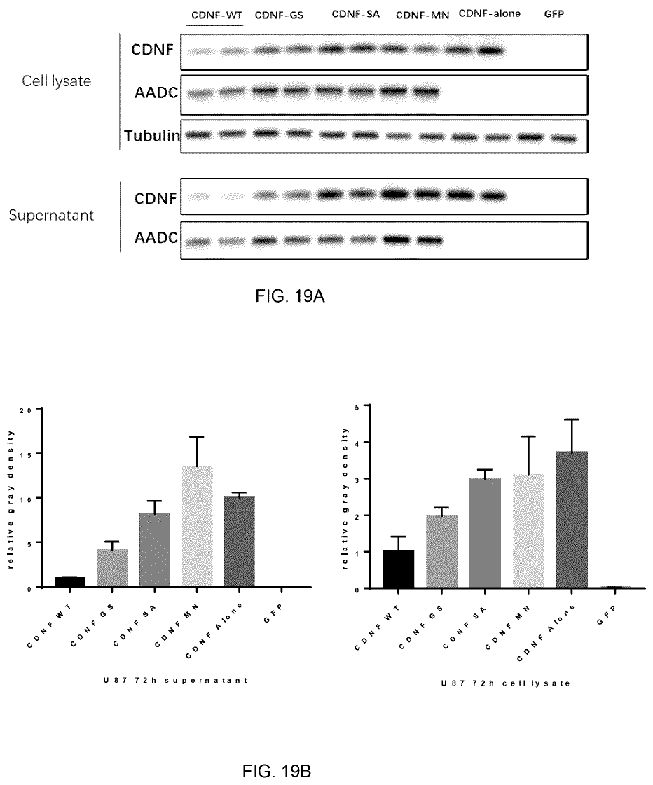

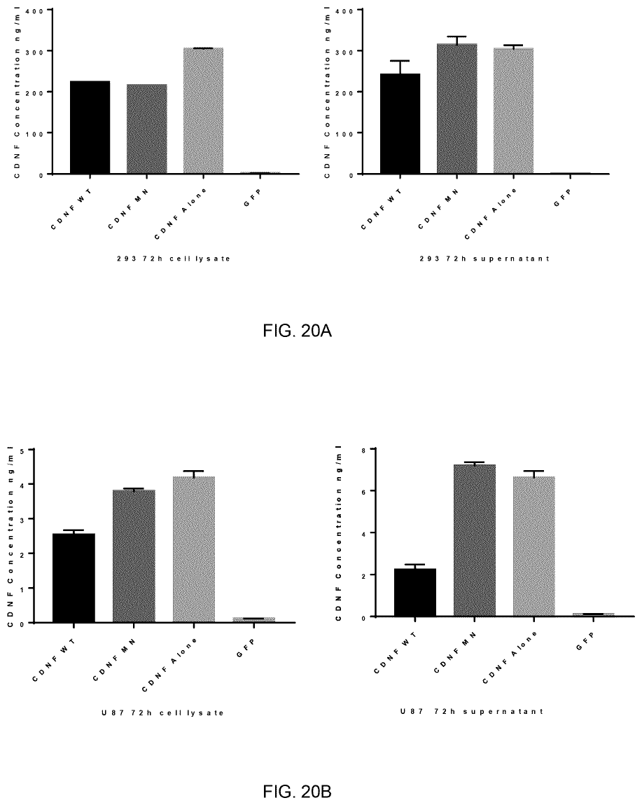

To conduct WB analysis, the samples were diluted and boiled in 5× loading buffer (Beyotime, P0015L), separated on SDS polyacrylamide gel (BIO-RAD,1703932) and transferred onto a 0.22 μm PVDF membrane (BIO-RAD, 1620177). The membrane was incubated with antibodies for CDNF protein (Sigma, HPA044587), AADC protein (Merck Millipore, AB1569), and β-Tubulin protein (Tubulin, Proteintech, 66240-1-Ig) overnight at 4° C., followed by 2 hours incubation with HRP-linked secondary antibody at room temperature the next day. BeyoECL Moon kit (Beyotime, P0018F) was then applied to the membrane (FIG. 18A). Exposure and image capture were performed with a Tannon 5600 system. The intensities of the CDNF bands were calculated and normalized to construct ApC-WT and the results are shown in FIG. 18B. As shown in FIG. 18B, among all of the constructs evaluated, cells transfected with construct ApC-MN showed the highest levels of CDNF protein secreted into the supernatant, which was confirmed in U-87 MG cells transfected with the same set of plasmids (FIG. 19A-B).

The levels of secreted CDNF protein in the supernatants were further determined by a sandwich ELISA method. Briefly, 96-well microplates (Costar, 42592) were coated with a CDNF mouse Mab (SinoBiological, 15691-MM1) antibody as a capture antibody at 4° C. overnight. The next day, the plates were washed 3 times with PBST (Phosphate Buffered Saline with Tween), then blocked with 5% Nonfat-Dried Milk (Solarbio, D8340) at room temperature for 2 hours. After an additional wash step, CDNF protein standard (SinoBiological, 15691-H08H) or protein samples were added to the plates in duplicates. The samples were incubated with the detection antibody (SinoBiological, 15691-R104) for 2 hours at room temperature, followed by a wash step. Then the samples were incubated with HRP-conjugated goat anti-rabbit antibody (SinoBiological, HO14SE1801) for 1 hour, followed by one more wash step. The bound HRP conjugates were detected by addition of TMB (Solarbio, PR1200), and the reaction was terminated by the stop solution (Solarbio, C1058). The absorbance of each sample (at an excitation wavelength of 450 nm and emission @630 nm) was detected by a fluorimeter (SPECTRAmax Gemini XPS, Molecular Devices, San Jose, CA, USA) and the results are shown in FIG. 20A-B.

As shown in FIG. 20A-B, both HEK293 and U-87 MG cells transfected with construct ApC-MN expressed significantly higher levels of CDNF protein as shown in the high levels of secreted CDNF in the supernatants. As a result, ApC-MN was selected for further evaluation.

### Example 10. Codon Optimized CDNF Expressed Protein Showed Significant Cytoprotective Effects

In this Example, the cytoprotective effect of CDNF protein expressed and secreted by cells transfected with the plasmid AADC-CDNF MN (ApC-MN) was investigated. Two cell-based assays, LDH cytotoxicity assay and CCK-8 cell viability assay of cells treated with MPP+ or Rotenone were used to examine the CDNF cytoprotective effect.

In the LDH assay, a 96-well assay plate containing SH-SY5Y cells (National Collection of Authenticated Cell Cultures, SCSP-5014) was pre-treated with or without supernatant containing secreted CDNF protein collected from the previous experiments. Several wells were left without cells as the blank control to determine the background luminescence levels. Cells were then treated with MPP+ or Rotenone or DMSO vehicle control. When the plate was incubated at 37° C., LDH Detection Reagent (CytoTox 96® Non-Radioactive Cytotoxicity Assay, Promega) was prepared by mixing 12 mL of Assay Buffer with a bottle of Substrate Mix. 1/10^{th} volume of 10× Lysis Solution was added to the positive control (Maximum LDH Release Control) wells 45 minutes before adding the CytoTox 96® Reagent. Before plate reading, 50 μL of the CytoTox 96® Reagent was added to each well. The plate was covered with foil to protect from light exposure and incubated for 30 min at room temperature. Then, 50 μL of Stop Solution was added to each well to stop the reaction. One hour after adding the Stop Solution, the absorbance at 490 nm was recorded by a plate reader. Percent cytotoxicity=100×Experimental LDH Release (OD490)/Maximum LDH Release (OD490).

In the CCK-8 assay, the SH-SY5Y cells in logarithmic growth phase were plated in 96 well plates at a density of 5×10³/mL and cultured in high glucose/DMEM cell culture medium containing 10% fetal bovine serum (FBS) and 1% penicillin-streptomycin solution and maintained in a cell incubator with 5% CO₂ and 95% air at 37° C. for 12 hrs. Cells were then treated with or without supernatant containing secreted CDNF collected from the previous experiments. MPP+ or Rotenone containing serum-free media at a final concentration from 0 to 2 mM were used to induce cell apoptosis. 24 hrs later, cell viability was assessed by adding 10 μL of CCK-8 (Cell Counting Kit-8, Dojindo) to the culture and after incubation for 2 hrs, relative cell viability was determined by a spectrometry @450 nm wavelength.

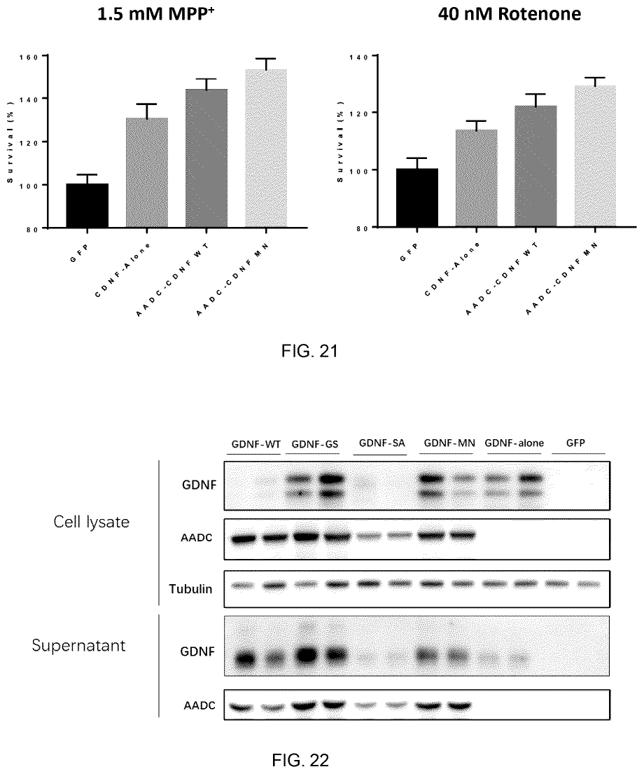

As shown in FIG. 21, in the presence of MPP+(1.5 mM) or Rotenone (40 nM), SH-SY5Y cells treated with supernatants from construct ApC-MN transfected cells had a higher survival rate than those treated with supernatants from construct ApC-WT transfected cells, indicating that in comparison to construct ApC-WT, construct ApC-MN expressed and secreted higher levels of CDNF protein to the supernatants to exert its stronger cytoprotective effect.

### Example 11. Codon-Optimized Human GDNF Sequences for AADC Combination Constructs

In this example, the mature protein coding sequence of human GDNF was optimized by codon optimization process as performed in Example 9 (FIG. 17B), obtaining GDNF-GS sequence (SEQ ID No: 27), GDNF-SA sequence (SEQ ID No:28) and GDNF-MN sequence (SEQ ID No:29).

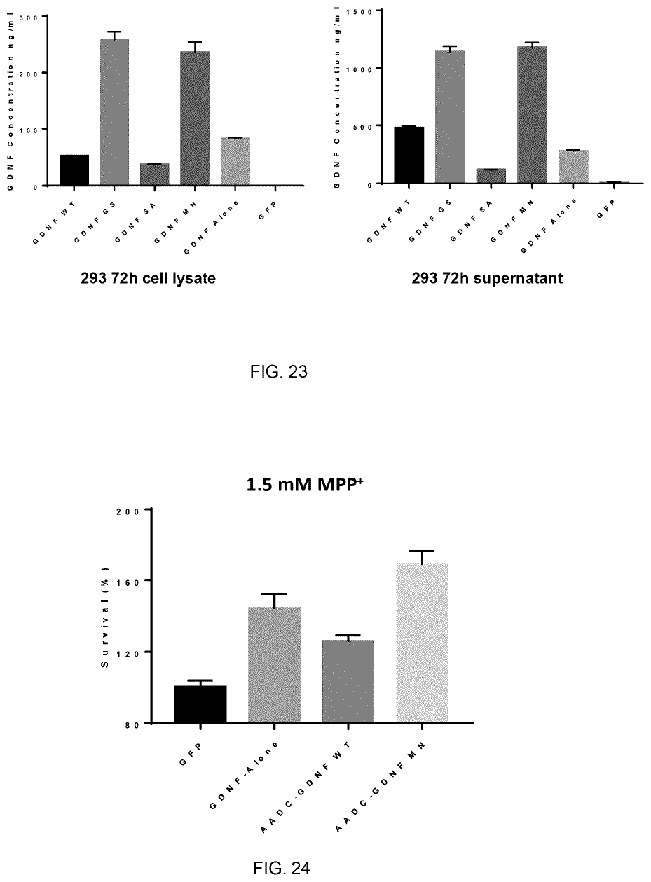

The same in vitro cellular experiments as described in Example 10 were performed in HEK293 cells transfected with constructs containing GDNF GS, GDNF SA and GDNF MN (FIGS. 22-24). Based on the results, the GDNF MN sequence with no CpG island had the best GDNF expression and protection potential among all the codon-optimized GDNF sequences evaluated.

Therefore, the GDNF MN sequence was chosen as the GDNF coding sequence for AADC combination construct for further evaluation.

### Example 12. Determination of the Optimal Promoters to Use in the AADC and CDNF/GDNF Combination Constructs

To determine the best promoter elements to use in the combination construct of AADC with either CDNF or GDNF, eight new combination constructs were made, specifically as shown in the Table 3 below. The four constructs containing CDNF are CAG-A3-P2A-CDNF MN, CAG-A11-P2A-CDNF MN, EF1α-A3-P2A-CDNF MN, and EF1α-A11-P2A-CDNF MN; and the four constructs containing GDNF are CAG-A3-P2A-GDNF MN, CAG-A11-P2A-GDNF MN, EF1α-A3-P2A-GDNF MN, and EF1α-A11-P2A-GDNF MN. Constructs CBh-AADC WT-P2A-CDNF WT and CBh-AADC WT-P2A-GDNF WT were generated and used as controls.

TABLE 3
Constructs of CDNF/GDNF and AADC in combination with
different regulatory sequences

| | | | | | |
|---|---|---|---|---|---|
| No. | Vector | Promoter | AADC | NTF | Results |

CDNF groups

| | | | | | |
|---|---|---|---|---|---|
| 1 | CAG-A3-P2A-CDNF MN | CAG | A3 | CDNF MN | Lower |
| 2 | CAG-A11-P2A-CDNF MN | CAG | A11 | CDNF MN | ND* |
| 3 | EF1α-A3-P2A-CDNF MN | EF1α | A3 | CDNF MN | ND* |
| 4 | EF1α-A11-P2A-CDNF | EF1α | A11 | CDNF MN | Higher |
| | MN | | | | |

GDNF groups

| | | | | | |
|---|---|---|---|---|---|
| 5 | CAG-A3-P2A-GDNF MN | CAG | A3 | GDNF MN | |
| 6 | CAG-A11-P2A-GDNF MN | CAG | A11 | GDNF MN | |
| 7 | EF1α-A3-P2A-GDNF MN | EF1α | A3 | GDNF MN | ND* |
| 8 | EF1α-A11-P2A-GDNF | EF1α | A11 | GDNF MN | High- |
| | MN | | | | est |

*N.D.: Non-detectable

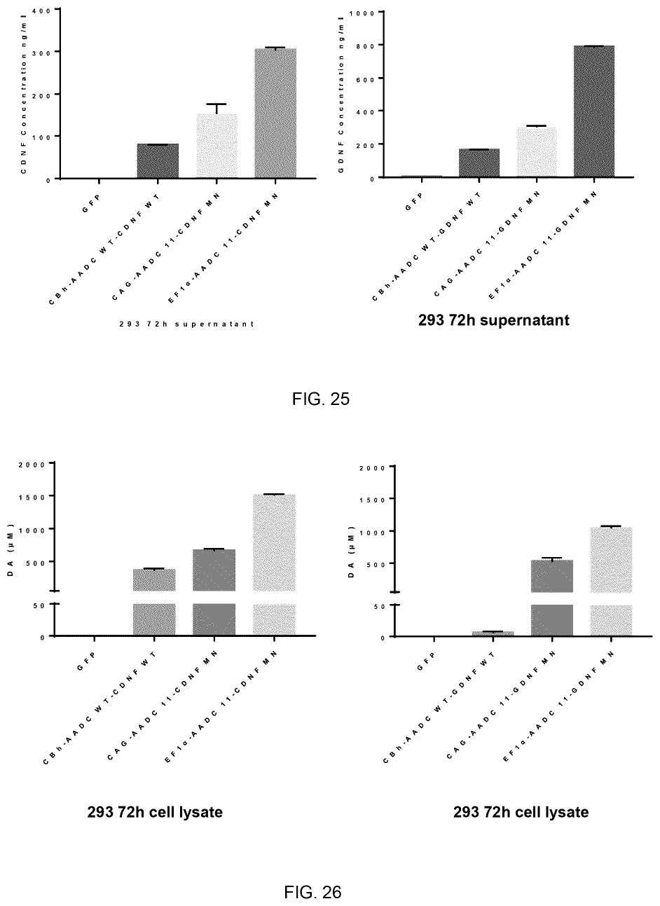

Aforementioned constructs were transfected into HEK293 cells. Both the cell lysates and supernatants of these samples were collected. The secreted protein levels of CDNF or GDNF were determined by ELISA. The results showed that EF1α-A11-P2A-CDNF MN expressed higher levels of CDNF protein than that of CAG-A3-CDNF MN, while the other two constructs expressed undetectable levels of CDNF protein (FIG. 25).

For GDNF containing constructs, EF1α-A11-GDNF MN expressed and secreted the highest levels of GDNF (FIG. 25).

The catalytic activities of AADC protein expressed by these combination constructs were also determined by HPLC assay of the cell lysates as described in the previous example. Shown in FIG. 26, the relative AADC activities of EF1α-A11-CDNF MN and EF1α-A11-GDNF MN were much higher than all of the other candidate constructs.

The construct EF1α-CDNF MN-A11 was generated by placing the CDNF codon before the AADC codon and linked by P2A.

Both EF1α-CDNF MN-A11 (CDNF MN-A11) and EF1α-A11-CDNF MN (A11-CDNF MN) were transfected into HEK293 cells by Lipofectamine 3000. 72 hours after transfection, the supernatant and cell lysate were collected. The protein level of AADC and CDNF in the cell lysate and CDNF level in the supernatant (secreted CDNF) were determined by WB. It was interesting to find that the protein level of secreted CDNF was much higher in the CDNF MN-A11 group than in A11-CDNF MN (FIG. 42).

### Example 13. Validation of the Cyto-Protective Effects of CDNF and GDNF Proteins Derived from Candidate Constructs

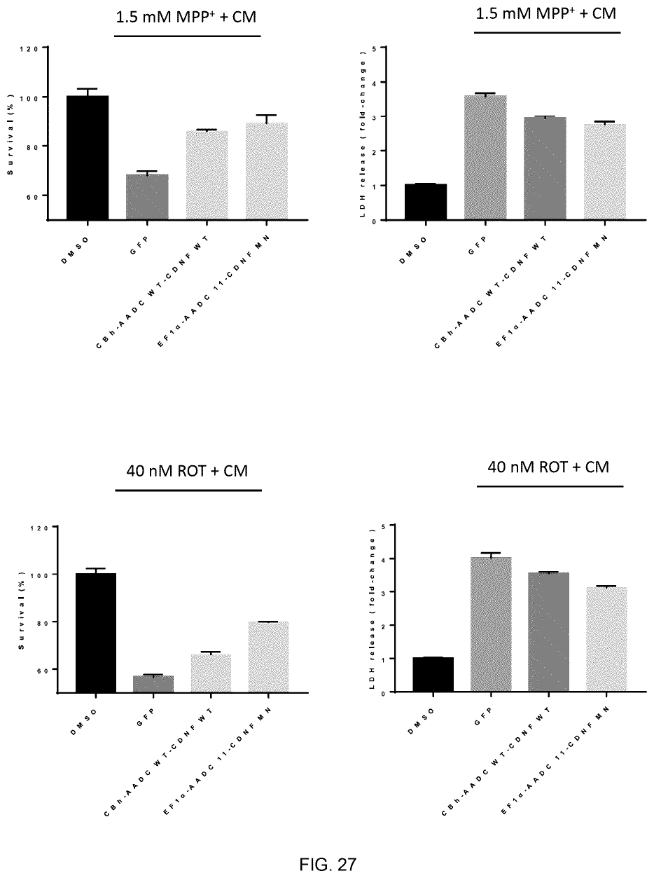

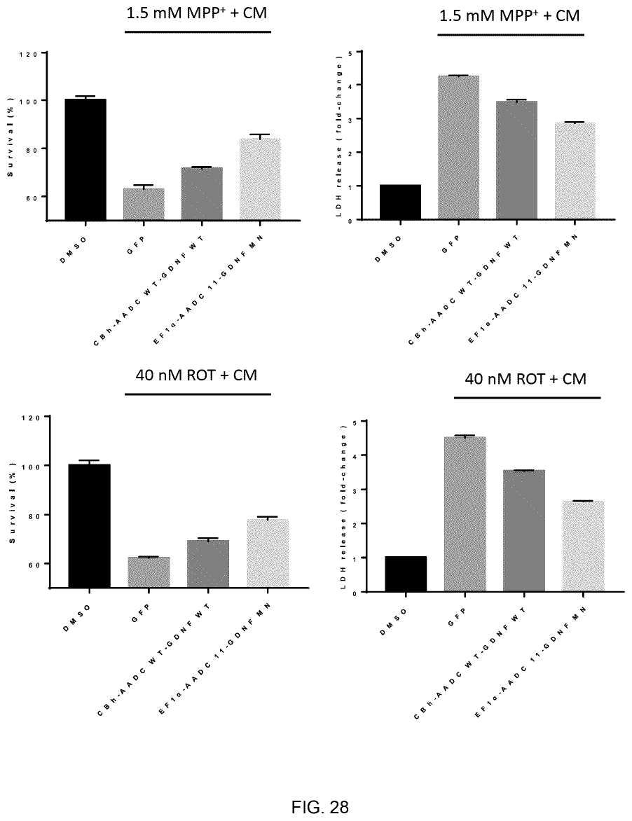

The MPP+ and Rotenone assays as described in the previous experiment were performed to determine the cytoprotective effects of the CDNF and GDNF constructs identified in Example 12. The supernatants from cells transfected with construct EF1α-A11-CDNF MN (FIG. 27) or EF1α-A11-GDNF MN (FIG. 28) improved the viability of SH-SY5Y cells in the presence of MPP+(1.5 mM) or Rotenone (40 nM). LDH assay was also performed to confirm the cyto-protective effects of both candidate constructs.

### Example 14. Evaluation of AADC and CDNF or GDNF Protein Expression of the AAV9 Packaged Combination Constructs

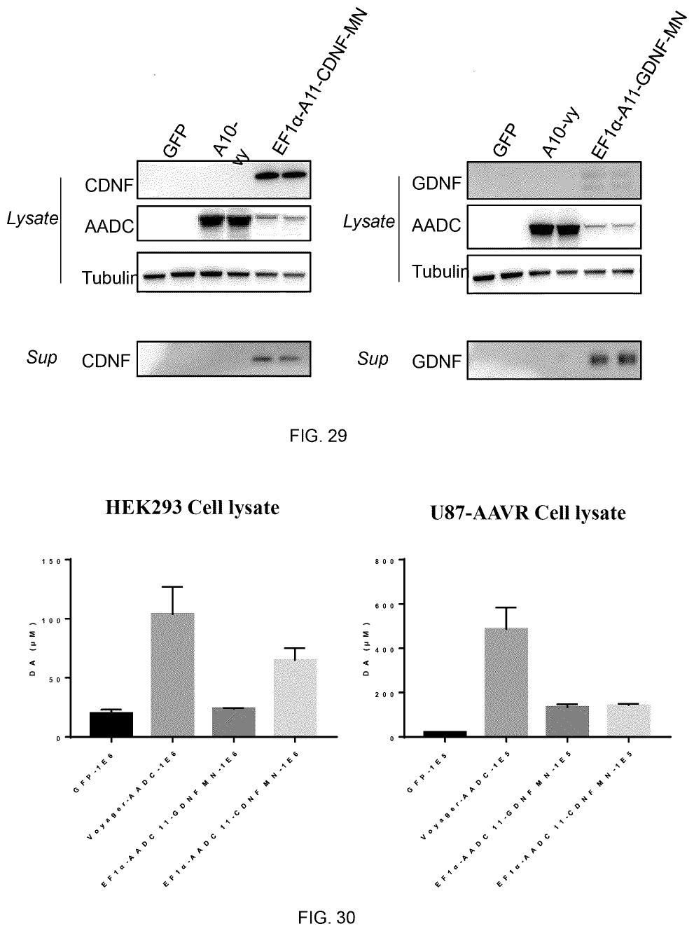

Both EF1α-A11-CDNF MN and EF1α-A11-GDNF MN combination constructs were packaged into AAV9. The obtained AAV9-EF1α-A11-CDNF MN and AAV9-EF1α-A11-GDNF MN were added to U87-AAVR cells at MOI=1e5. At 72 hours post viral transduction, the cells were collected in the lysis buffer. Following the protocol of WB, the expressed protein levels of CDNF and AADC were determined. Meanwhile, the supernatants of all the samples were also collected and concentrated as above mentioned. The levels of secreted CDNF and GDNF proteins were also detected by WBs and the results are shown in FIG. 29.

The relative activities of the expressed AADC protein in the aforementioned cell lysates were shown in FIG. 30.

Taken together data shown in FIGS. 29 and 30, both constructs performed as expected when packaged into AAV9 as rAAV.

### Example 15. In Vivo Therapeutic Efficacy of AAV Candidates Expressing Optimized AADC in Combination with GDNF or CDNF in PD Mouse Models

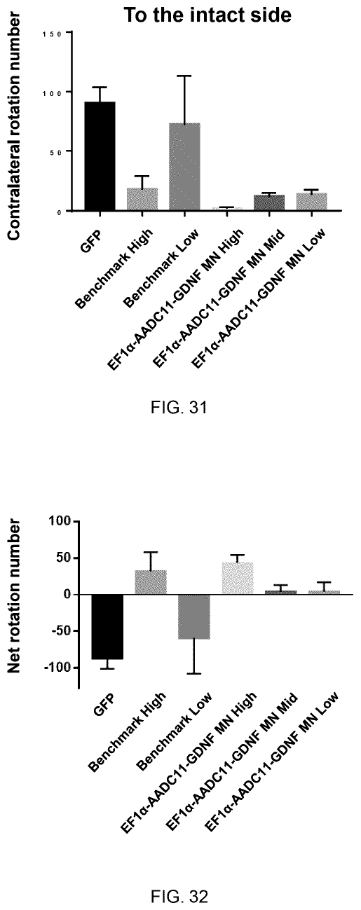

The therapeutic effects of AAV delivering the optimized AADC+GDNF combination construct were tested in a unilateral-lesioned 6-OHDA PD mouse model. Briefly, C57 mice were placed in the stereotaxic frame and anesthetized with the isoflurane mixed in oxygen. 1 μL 6-OHDA (3 mg/ml, Sigma, #162957) was injected into the right substantial nigra (coordinates AP=−2.9 mm, ML=−1.1 mm, DV=−4.5 mm). One week after the lesion surgery, the mice were administrated with apomorphine (Sigma, PHMR2621-500MG) intraperitoneally, and their rotation behavior (rotating to the intact side) was recorded and analyzed. Candidate rAAV vector EF1α-A11-GDNF MN was then injected into the right striatum of the subjects (coordinates AP=+0.6 mm, ML=−1.8 mm, DV=−3.2 mm) at three doses (Low: 1E+12 vg, Medium: 3.16E+12 vg, High: 1E+13 vg in mL). AAV-GFP and two doses of benchmark AAV-A10-vy (Low: 1E+12 vg, High: 1E+13 vg in mL) were also applied. 21 days post AAV injection, it was observed that the contralateral rotation triggered by the apomorphine was significantly decreased in all of the study groups treated with the candidate rAAV vector as compared to the control group (FIG. 31 and FIG. 32). As shown in FIG. 31, the three study groups treated with the three different doses of EF1α-A11-GDNF MN rAAV performed significantly better than the group treated with the benchmark rAAV More remarkably, the net rotation numbers (ipsilateral-contralateral) in both low and medium dose treatment groups were close to zero which indicated that these study subjects had fully recovered their balance motor behavior, while the group received the high dose of benchmark showed over-compensated ipsilateral rotation (an excess amount of AADC in the treated side) (FIG. 32).

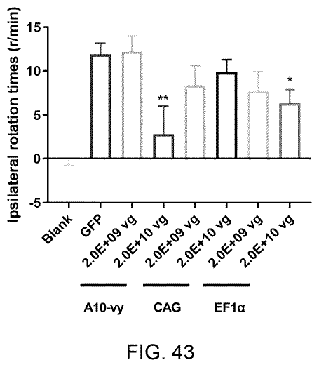

In another in vivo efficacy test in PD mice (study 2), CAG-A11-GDNF MN rAAV (AAV9) was also tested in the unilateral-lesioned 6-OHDAPD mouse model. Two different doses (2.0E+09 vg and 2.0E+10 vg) of both CAG-A11-GDNF MN and EF1α-A11-GDNF MN were delivered into the PD mice via intra-striatal injection. AAV-GFP was used as untreated control and two doses of AAV2-A10-vy was used as the benchmark. 21 days post AAV injection, it was found that 2E+10 vg/animal of EF1α-A11-GDNF MN significantly decreased the ipsilateral rotation triggered by the amphetamine (i.p. 5 mg/kg), indicating a rescued motor function (FIG. 43).

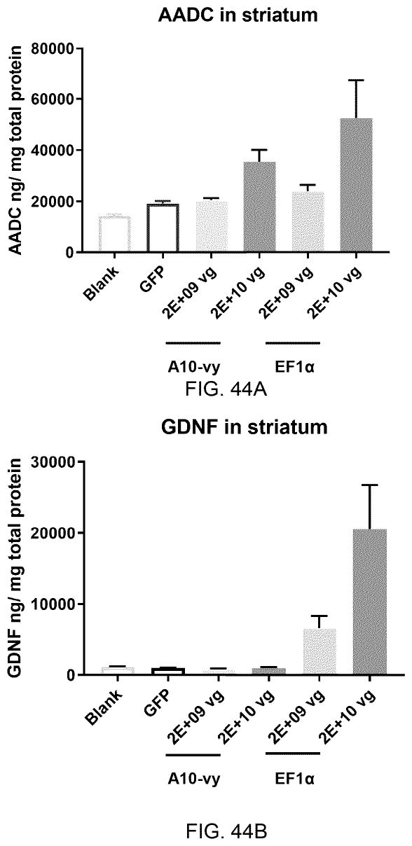

The tissue samples were collected for determination of the expression of AADC and NTF proteins, and dopamine levels. Expression of the GOIs were observed in the Nigro-striatal pathway. As shown in FIG. 44, striatum tissue samples were collected from the subjects in 6-OHDA mice study 2 on day 91 post AAV injection. The protein levels of AADC and GDNF in both EF1α and benchmark groups were determined by WB. It was surprised to find that EF1α expressed more AADC in the striatum (the injection site) than A10-vy (FIG. 44A). Meanwhile, it was confirmed that EF1α expressed GDNF dose-dependently in vivo (FIG. 44B).

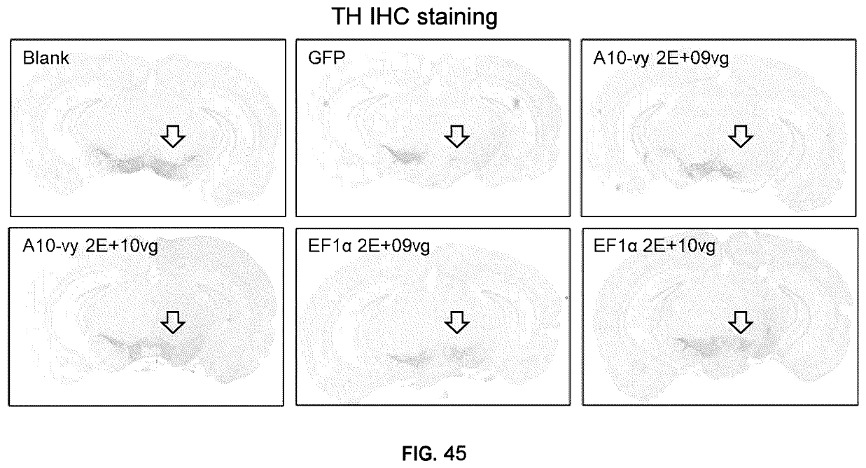

Moreover, tyrosine hydroxylase (TH) staining showed more dopaminergic terminals survived in the Nigro-striatal pathway of the EF1α treated animals as compared to the control animals which only received AAV9-GFP, confirming the protective effects of the candidate rAAVs (FIG. 45).

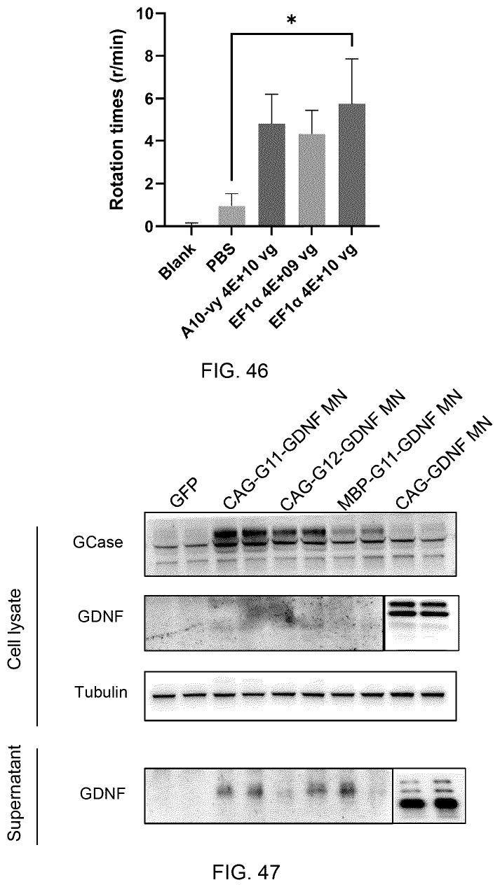

The efficacy of AAV9-EF1α-A11-GDNF MN (EF1α) was also tested in a unilateral-lesioned 6-OHDA PD rat model. The model was generated according to the previous study (6-OHDA Lesion Models of Parkinson's Disease in the Rat. Animal Models of Movement Disorders: volume I, Neuromethods, vol. 61, DOI 10.1007/978-1-61779-298-4_13). Briefly, 6-OHDA was injected into the right substantial nigra (coordinates AP=−4.4 mm, ML=−1.1 mm, DV=−8 mm). Two weeks after the lesion surgery, EF1α was then injected into two sites of the right striatum of the subjects (coordinates AP=+1.0 mm, ML=−3.0 mm, DV=−4.5 mm; AP=−0.2 mm, ML=−3.5 mm, DV=−5.0 mm) at two doses (Low: 4.0E+9 vg/animal, High: 4E+10 vg/animal). One dose of benchmark AAV2-A10-vy (4E+10 vg/animal) was also applied. 21 days post AAV injection, the spontaneous contralateral rotation behavior was recorded pre and post L-DOPA (5 mg/kg L-DOPA+2.5 mg/kg Benserazide) administration. Δrotation time per minute (Post-Pre) represented the response of subjects to L-DOPA. As shown in FIG. 46, it was found that EF1α significantly enhanced the response of the treated subjects to low dose L-DOPA, suggesting in the future clinical application, this candidate treatment could enhance the bioavailability of dopamine derivative like L-DOPA. In addition, EF1a showed a better result as compared to A10-vy at the same dosing level (4E+10 vg).

### Example 16. In Vivo Therapeutic Efficacy of AAV Candidates Expressing Optimized GBA1 in Combination with CDNF or GBA1 with GDNF in the MSA Animal Models

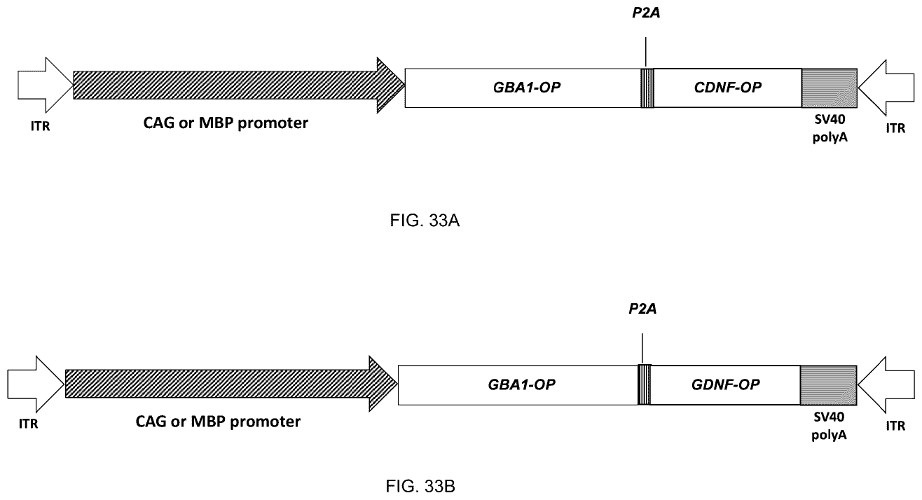

The candidate constructs with configuration as shown in FIGS. 33A and 33B were generated. These constructs contain a CAG or MBP promoter, GBA1 gene (including SEQ ID NOs: 45 and 47) in combination with CDNF (SEQ ID NOs: 23-25) or GDNF (SEQ ID NOs: 27-29).

CAG-G11-P2A-GDNF MN-SV40 polyA (CAG-G11-GDNF MN), CAG-G12-P2A-GDNF MN-SV40 polyA (CAG-G12-GDNF MN), MBP-G11-P2A-GDNF MN-bGH polyA (MBP-G11-GDNF MN), and CAG-GDNF MN-WPRE-hGH (CAG-GDNF MN) were constructed and packaged into AAVs in which serotype AAV9 was used. These candidate rAAV vectors were applied on the U-87 MG-AAVR cells (U-87 MG cells which overexpressed AAVR) and the cell lysate and supernatant samples were collected. The GCase and GDNF protein levels were determined in these samples (FIG. 47).

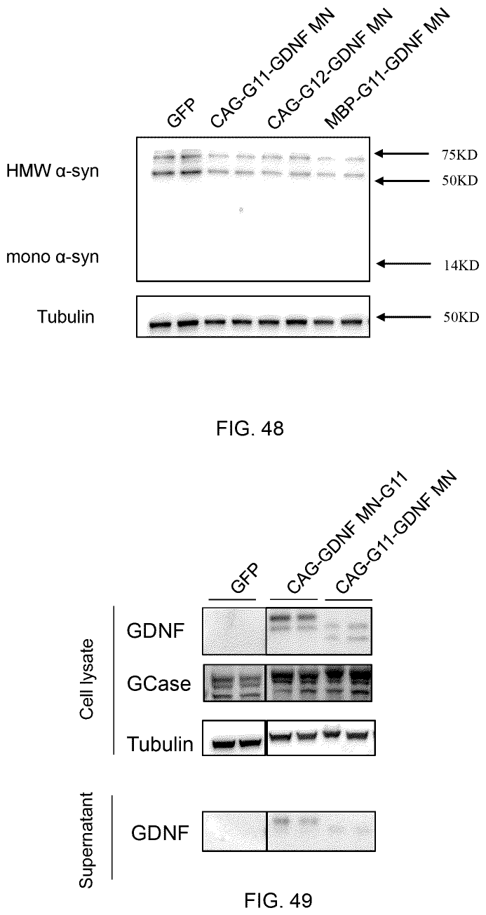

The efficacy of the above candidate rAAV vectors were tested in vitro in a cell model for recapitulating the pathological feature of PD and MSA, which is a stable cell line based on SH-SY5Y cell and over-expressing both AAVR and the mutated α-synuclein (point mutation A53T) protein. The lentivirus vectors harboring AAVR sequence or mutated α-synuclein (A53T) sequence, respectively were prepared by Azenta Life Science and applied to SH-SY5Y cells sequentially. After antibiotic- and GFP-based selection, the SH-SY5Y-AAVR-A53T stable cell line was generated. As shown in FIG. 48, it was found that candidate rAAV vectors could significantly decrease the level of α-synuclein at high molecular weight (HMW) which was the biomarker for α-syn aggregation in the cell model.

CAG-GDNF MN-P2A-G11-SV40 polyA (CAG-GDNF MN-G11) was constructed and transfected into HEK293 cells, together with CAG-G11-GDNF MN. 72 hours post transfection, the supernatant and cell lysate samples were collected and analyzed. It was found that CAG-GDNF MN-G11 expressed more GDNF compared to CAG-G11-GDNF MN in both supernatant and cell lysate, and surprisingly maintain the capability to express GCase (FIG. 49).

Then the therapeutic effects of AAV delivering the optimized GBA1+CDNF or GBA1+GDNF combination construct are evaluated in the mouse models of MSA. Candidate rAAV vectors after injection into the lateral ventricle (i.c.v.) rescue the motor deficiency in MSA animals. The tissue samples are collected. The expression of GBA1 and NTF proteins, and the reduced levels of α-syn and phosphorylated α-syn in the Nigro-striatal pathway are observed. Moreover, tyrosine hydroxylase (TH) staining shows more dopaminergic terminals survived in the striatum of the treated animals confirming the protective effects of the candidate AAVs.

### Example 17. In Vivo Therapeutic Efficacy of AAV Candidates Expressing Optimized GBA1 in PD Mouse Models

The therapeutic effects of the AAVs expressing optimized GBA1 are tested in PD mouse models.

The mice which are genetically engineered to overexpress mutated α-synuclein (point mutation A53T) are used as the PD model. CAG-G11-hGH polyA (as shown in Example 8 and FIG. 38A) is packaged into AAV9 to generate rAAV vector. The candidate rAAV vector is injected into the lateral ventricle of the A53T PD mouse model to evaluate its effect. The rAAV9-CAG-G11-hGH can significantly relieve the motor deficiency in the PD mouse model and reduce the levels of α-syn and phosphorylated α-syn in the Nigro-striatal pathway indicating its potential in treating PD and other α-syn proteinpathies.

SEQUENCE INFORMATION

| | | | CODE |
|---|---|---|---|
| ID | TYPE | SEQUENCE | NAME |
| 1 | DNA | [symbol] | AADC- |
| | | [symbol] | A1 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 2 | DNA | [symbol] | AADC- |
| | | [symbol] | A2 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 3 | DNA | [symbol] | AADC- |
| | | [symbol] | A3 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 4 | DNA | [symbol] | AADC- |
| | | [symbol] | A4 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 5 | DNA | [symbol] | AADC- |
| | | [symbol] | A5 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 6 | DNA | [symbol] | AADC- |
| | | [symbol] | A6 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 7 | DNA | [symbol] | AADC- |
| | | [symbol] | A7 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 8 | DNA | [symbol] | AADC- |
| | | [symbol] | A8 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 9 | DNA | [symbol] | AADC- |
| | | [symbol] | A9 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 10 | DNA | [symbol] | AADC- |
| | | [symbol] | A10 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 11 | DNA | [symbol] | AADC- |
| | | [symbol] | A0 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 12 | DNA | [symbol] | GBA1- |
| | | [symbol] | G1 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 13 | DNA | [symbol] | GBA1- |
| | | [symbol] | G2 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 14 | DNA | [symbol] | GBA1- |
| | | [symbol] | G3 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 15 | DNA | [symbol] | GBA1- |
| | | [symbol] | G4 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 16 | DNA | [symbol] | GBA1- |
| | | [symbol] | G5 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 17 | DNA | [symbol] | GBA1- |
| | | [symbol] | G6 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 18 | DNA | [symbol] | GBA1- |
| | | [symbol] | G7 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 19 | DNA | [symbol] | GBA1- |
| | | [symbol] | G8 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 20 | DNA | [symbol] | GBA1- |
| | | [symbol] | G9 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 21 | DNA | [symbol] | GBA1- |
| | | [symbol] | G10 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 22 | DNA | [symbol] | GBA1- |
| | | [symbol] | G0 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 23 | DNA | [symbol] | CDNF |
| | | [symbol] | GS |
| | | [symbol] | |
| | | [symbol] | |
| | | [symbol] | |
| | | [symbol] | |
| 24 | DNA | [symbol] | CDNF |
| | | [symbol] | SA |
| | | [symbol] | |
| | | [symbol] | |
| | | [symbol] | |
| | | [symbol] | |
| 25 | DNA | [symbol] | CDNF |
| | | [symbol] | MN |
| | | [symbol] | |
| | | [symbol] | |
| | | [symbol] | |
| | | [symbol] | |
| 26 | DNA | [symbol] | CDNF- |
| | | [symbol] | WT |
| | | [symbol] | |
| | | [symbol] | |
| | | [symbol] | |
| | | [symbol] | |
| 27 | DNA | [symbol] | GDNF |
| | | [symbol] | GS |
| | | [symbol] | |
| | | [symbol] | |
| | | [symbol] | |
| | | [symbol] | |
| | | [symbol] | |
| 28 | DNA | [symbol] | GDNF |
| | | [symbol] | SA |
| | | [symbol] | |
| | | [symbol] | |
| | | [symbol] | |
| | | [symbol] | |
| | | [symbol] | |
| 29 | DNA | [symbol] | GDNF |
| | | [symbol] | MN |
| | | [symbol] | |
| | | [symbol] | |
| | | [symbol] | |
| | | [symbol] | |
| | | [symbol] | |
| 30 | DNA | [symbol] | GDNF-WT |
| | | [symbol] | |
| | | [symbol] | |
| | | [symbol] | |
| | | [symbol] | |
| | | [symbol] | |
| | | [symbol] | |
| 31 | PRT | [symbol] | AADC |
| | | [symbol] | |
| | | [symbol] | |
| | | [symbol] | |
| | | [symbol] | |
| 32 | PRT | [symbol] | GBA1 |
| | | [symbol] | |
| | | [symbol] | |
| | | [symbol] | |
| | | [symbol] | |
| | | [symbol] | |
| 33 | PRT | [symbol] | CDNF |
| | | [symbol] | |
| 34 | PRT | [symbol] | GDNF |
| | | [symbol] | |
| | | [symbol] | |
| 35 | DNA | [symbol] | E2A |
| 36 | PRT | [symbol] | E2A |
| 37 | PRT | [symbol] | F2A |
| 38 | PRT | [symbol] | F2A |
| 39 | DNA | [symbol] | T2A |
| 40 | PRT | [symbol] | T2A |
| 41 | DNA | [symbol] | P2A |
| 42 | PRT | [symbol] | P2A |
| 43 | DNA | [symbol] | [symbol] |
| 44 | DNA | [symbol] | EMCV |
| | | [symbol] | IRES |
| | | [symbol] | |
| | | [symbol] | |
| | | [symbol] | |
| | | [symbol] | |
| 45 | DNA | [symbol] | GBA1- |
| | | [symbol] | G11 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 46 | DNA | [symbol] | AADC |
| | | [symbol] | A11 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 47 | DNA | [symbol] | GBA1- |
| | | [symbol] | G12 |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 48 | DNA | [symbol] | A10- |
| | | [symbol] | vy |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 49 | DNA | [symbol] | G10- |
| | | [symbol] | p |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 50 | DNA | [symbol] | [symbol] |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 51 | DNA | [symbol] | [symbol] |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 52 | DNA | [symbol] | [symbol] |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 53 | DNA | [symbol] | [symbol] |
| | | [symbol] | |
| | | [symbol] | |
| | | [symbol] | |
| | | [symbol] | |
| | | [symbol] | |
| | | [symbol] | |
| | | [symbol] | |
| 54 | DNA | [symbol] | [symbol] |
| | | [symbol] | |
| | | [symbol] | |
| | | [symbol] | |
| | | [symbol] | |
| | | [symbol] | |
| | | [symbol] | |
| | | [symbol] | |
| 55 | DNA | [symbol] | [symbol] |
| | | [symbol] | |
| | | [symbol] | |
| | | [symbol] | |
| | | [symbol] | |
| | | [symbol] | |
| 56 | DNA | [symbol] | [symbol] |
| | | [symbol] | |
| | | [symbol] | |
| | | [symbol] | |
| | | [symbol] | |
| | | [symbol] | |
| 57 | DNA | [symbol] | EF1α |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 58 | DNA | [symbol] | CAG |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 59 | DNA | [symbol] | MBP |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| | | [symbol] | |
| 60 | DNA | [symbol] | [symbol] |
| | | [symbol] | |
| | | [symbol] | |
| | | [symbol] | |
| | | [symbol] | |
| | | [symbol] | |
| | | [symbol] | |
| | | [symbol] | |

[symbol]

indicates data missing or illegible when filed

1. An isolated nucleic acid molecule, comprising a first nucleotide sequence encoding a first protein operatively linked to a second nucleotide sequence encoding a second protein, wherein the first protein and the second protein are selected from the following combination (a) or (b):
(a) the first protein is human AADC, and the second protein is human GDNF; or
(b) the first protein is human GDNF, and the second protein is human AADC,
wherein the first nucleotide sequence is at 5′ upstream of the second nucleotide sequence.

2. The isolated nucleic acid molecule of claim 1, wherein:
the polypeptide sequence of AADC comprises or is the amino acid sequence as shown in SEQ ID NO: 31; and/or
the polypeptide sequence of GDNF comprises or is the amino acid sequence as shown in SEQ ID NO: 34.

3. The isolated nucleic acid molecule of claim 1, wherein when the first protein is human ADCC, the first nucleotide sequence comprises or is a nucleotide sequence selected from a group consisting of a nucleotide sequence as shown in any one of SEQ ID NOs: 1-9, 11 or 46.

4. The isolated nucleic acid molecule of claim 1, when the second protein is human GDNF, wherein the second nucleotide sequence comprises or is a nucleotide sequence selected from a group consisting of a nucleotide sequence as shown in any one of SEQ ID NOs: 27-30.

5. The isolated nucleic acid molecule of claim 1, wherein the nucleotide sequence encoding human GDNF is a codon-optimized coding sequence of GDNF, and the codon-optimized coding sequence differs from the wild-type coding sequence of GDNF as shown in SEQ ID NO: 30 in nucleotides not present in the region coding for the signal peptide or pre-peptide of GDNF.

6. The isolated nucleic acid molecule of claim 5, wherein the nucleotide sequence encoding human GDNF is a codon-optimized coding sequence of GDNF and has reduced CpG sites or has no CpG islands as compared to the wild-type coding sequence of GDNF as shown in SEQ ID NO: 30.

7. The isolated nucleic acid molecule of claim 1, wherein the first nucleotide sequence and the second nucleotide sequence are linked in frame and are operatively linked to a promoter located at the 5′ upstream to both the first and the second nucleotide sequences.

8. The isolated nucleic acid molecule of claim 7, wherein the promoter is selected from a CBh promoter, an EF1α promoter, a CAG promoter, a MBP promoter or a variant thereof.

9. The isolated nucleic acid molecule of claim 8, wherein the promoter is a truncated variant of EF1α promoter having a nucleotide sequence of any one of SEQ ID NOS: 50-56.

10. A codon-optimized sequence of AADC, having a nucleotide sequence as shown in any one of SEQs ID NO: 1-9 or 46.

11. A codon-optimized sequence of GBA1, having a nucleotide sequence as shown in any one of SEQ ID NOs: 12-20, 45 or 47.

12. A codon-optimized sequence of GDNF, having a nucleotide sequence as shown in any one of SEQ ID NOs: 27-29.

13. A recombinant adeno-associated viral (rAAV) vector, comprising the isolated nucleic acid molecule of claim 1.

14. A viral particle comprising the rAAV vector of claim 13.

15. The viral particle of claim 14, which comprises a capsid protein of AAV1, AAV2, AAV5, AAV8 or AAV9.

16. A pharmaceutical composition, comprising the viral particle of claim 14, and a pharmaceutically acceptable excipient.

17. A method of treating or preventing neurodegenerative disorders in a subject in need thereof, comprising administering to the subject a therapeutically effective amount of the rAAV vector of claim 13.

18. The method of claim 17, wherein the neurodegenerative disorder is Parkinson's disease or AADC deficiency (AADCD).

19. A recombinant adeno-associated viral (rAAV) vector comprising the codon-optimized sequence of GBA1 of claim 11.

20. A method of treating or preventing neurodegenerative disorders in a subject in need thereof, comprising administering to the subject a therapeutically effective amount of the rAAV vector of claim 19, wherein the neurodegenerative disorder is Gaucher's disease (GD) or Multiple system atrophy (MSA).
